SEQUENCE LISTING (79 sequences)

SEQ ID NO: 1 - protein, 925 aa, synthetic construct
  REGION 1..925: note = ENPP1 Amino Acid Sequence - Wild Type
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 1077 aa, synthetic construct
  REGION 1..1077: note = Azurocidin-ENPP1-FC
  SIGNAL 19..20: note = Cleavage point of the signal sequence
  REGION 851..1077: note = MISC_FEATURE - Indicate Fc sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 1468 aa, synthetic construct
  REGION 1..1468: note = Azurocidin-ENPP1-Alb
  SIGNAL 19..20: note = Cleavage point of the signal sequence
  REGION 851..1468: note = MISC_FEATURE - Indicate Albumin sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 4 - protein, 1594 aa, synthetic construct
  REGION 1..1594: note = Azurocidin-ENPP1
  SIGNAL 19..20: note = Cleavage point of the signal sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 5 - protein, 888 aa, synthetic construct
  REGION 1..888: note = ENPP2 Amino Acid Sequence - Wild Type
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 6 - protein, 827 aa, synthetic construct
  REGION 1..827: note = Extracellular Domain of ENPP3
EKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 7 - protein, 875 aa, synthetic construct [sequence omitted]

SEQ ID NO: 8 - protein, 1073 aa, synthetic construct
  REGION 1..1073: note = Azurocidin-ENPP3-FC
  SIGNAL 19..20: note = Cleavage point of the signal sequence
  REGION 847..1073: note = MISC_FEATURE - Indicate Fc sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1464 aa, synthetic construct
  REGION 1..1464: note = Azurocidin-ENPP3-Albumin
  SIGNAL 19..20: note = Cleavage point of the signal sequence
  REGION 847..1464: note = MISC_FEATURE - Indicate Albumin sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 10 - protein, 846 aa, synthetic construct
  REGION 1..846: note = Azurocidin-ENPP3
  SIGNAL 19..20: note = Cleavage point of the signal sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 11 - protein, 453 aa, synthetic construct
  REGION 1..453: note = ENPP4 Amino Acid Sequence - Wild Type
MKLLVILLFSGLITGFRSDSSSSLPPKLLLVSFDGFRADYLKNYEFPHLQNFIKEGVLVEHVKNVFITKTFPNHYSIVTGLYEESHGIVANSMYDAVTKKHFSDSNDKDPFWWNEAVPIWVTNQLQENRSSAAAMWPGTDVPIHDTISSYFMNYNSSVSFEERLNNITMWLNNSNPPVTFATLYWEEPDASGHKYGPEDKENMSRVLKKIDDLIGDLVQRLKMLGLWENLNVIITSDHGMTQCSQDRLINLDSCIDHSYYTLIDLSPVAAILPKINRTEVYNKLKNCSPHMNVYLKEDIPNRFYYQHNDRIQPIILVADEGWTIVLNESSQKLGDHGYDNSLPSMHPFLAAHGPAFHKGYKHSTINIVDIYPMMCHILGLKPHPNNGTFGHTKCLLVDQWCINLPEAIAIVIGSLLVLTMLTCLIIIMQNRLSVPRPFSRLQLQEDDDDPLIG

SEQ ID NO: 12 - protein, 850 aa, synthetic construct
  REGION 1..850: note = ENPP51 Amino Acid Sequence
  SIGNAL 24..25: note = Cleavage point of the signal sequence
  SIGNAL 24..25: note = Cleavage point of the signal peptide sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 13 - protein, 1474 aa, synthetic construct
  REGION 1..1474: note = ENPP51 - ALB Amino Acid Sequence
  SIGNAL 24..25: note = Cleavage point of the signal peptide sequence
  REGION 857..1474: note = MISC_FEATURE - Indicate Albumin sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 14 - protein, 1074 aa, synthetic construct
  REGION 1..1074: note = ENPP5-NPP3-Fc sequence
  SIGNAL 22..23: note = Cleavage point of the signal peptide sequence
  REGION 848..1074: note = Indicate albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 1469 aa, synthetic construct
  REGION 1..1469: note = ENPP5-NPP3-Albumin sequence
  SIGNAL 22..23: note = Cleavage point of the signal peptide sequence
  REGION 862..1469: note = Indicate albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 16 - protein, 23 aa, synthetic construct
  REGION 1..23: note = ENPP5 Protein Export Signal Sequence
  VARIANT 23
MTSKFLLVSFILAALSLSTTFSX

SEQ ID NO: 17 - protein, 1079 aa, synthetic construct
  REGION 1..1079: note = ENPP51-Fc
  SIGNAL 22..23: note = Cleavage point of the signal peptide sequence
  REGION 853..1079: note = MISC_FEATURE - Indicate Fc sequence
MTSKFLLVSFILAALSLSTTFSGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 1082 aa, synthetic construct
  REGION 1..1082: note = ENPP71-Fc Amino Acid Sequence
  SIGNAL 22..23: note = Cleavage point of the signal peptide sequence
  REGION 856..1082: note = MISC_FEATURE - Indicate Fc sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 849 aa, synthetic construct
  REGION 1..849: note = ENPP71 (lacking NPP1 N-Terminus GLK) Amino Acid Sequence
  SIGNAL 22..23: note = Cleavage point of the signal peptide sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 20 - protein, 1079 aa, synthetic construct
  REGION 1..1079: note = ENPP71 (lacking NPP1 N-Terminus GLK) - Fc Amino Acid Sequence
  SIGNAL 22..23: note = Cleavage point of the signal peptide sequence
  REGION 853..1079: note = MISC_FEATURE - Indicate Fc sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 1474 aa, synthetic construct
  REGION 1..1474: note = - ENPP71 (lacking NPP1 N-Terminus GLK) - ALB Amino Acid Sequence
  SIGNAL 22..23: note = Cleavage point of the signal peptide sequence
  REGION 857..1474: note = MISC_FEATURE - Indicate Albumin sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 22 - protein, 1072 aa, synthetic construct
  REGION 1..1072: note = ENPP7-NPP3-Fc sequence
  SIGNAL 20..21: note = Cleavage point of the signal peptide sequence
  REGION 846..1072: note = MISC_FEATURE - Indicate Fc sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 1396 aa, synthetic construct
  REGION 1..1396: note = ENPP71-Albumin
  SIGNAL 23..24: note = Cleavage point of the signal peptide sequence
  REGION 857..1396: note = MISC_FEATURE - Indicate Fc sequence
MRGPAVLLTVALATLLAPGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTL

SEQ ID NO: 24 - protein, 1467 aa, synthetic construct
  REGION 1..1467: note = ENPP7-NPP3-Albumin
  SIGNAL 20..21: note = Cleavage point of the signal peptide sequence
  REGION 860..1467: note = MISC_FEATURE - Indicate Albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 25 - protein, 1507 aa, synthetic construct
  REGION 1..1507: note = ENPP7-ENPP3-Albumin
  SIGNAL 20..21: note = Cleavage point of the signal peptide sequence
  REGION 900..1507: note = Indicate albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 26 - protein, 852 aa, synthetic construct
  REGION 1..852: note = ENPP71 Amino Acid Sequence
  SIGNAL 22..23: note = Cleavage point of the signal peptide sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 27 - protein, 925 aa, synthetic construct
  REGION 1..925: note = ENPP121 Amino Acid Sequence
  SIGNAL 92..93: note = Cleavage point of the signal peptide sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 28 - protein, 1155 aa, synthetic construct
  REGION 1..1155: note = ENPP121-Fc Amino Acid Sequence
  SIGNAL 92..93: note = Cleavage point of the signal peptide sequence
  REGION 929..1155: note = MISC_FEATURE - Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 1550 aa, synthetic construct
  REGION 1..1550: note = ENPP121-ALB Amino Acid Sequence
  SIGNAL 92..93: note = Cleavage point of the signal peptide sequence
  REGION 933..1550: note = MISC_FEATURE - Indicate Albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 30 - protein, 1147 aa, synthetic construct
  REGION 1..1147: note = ENPP121-NPP3-Fc sequence
  SIGNAL 95..96: note = Cleavage point of the signal peptide sequence
  REGION 921..1147: note = Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 1542 aa, synthetic construct
  REGION 1..1542: note = ENPP121-NPP3-Albumin sequence
  SIGNAL 95..96: note = Cleavage point of the signal peptide sequence
  REGION 935..1542: note = MISC_FEATURE - Indicate Albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 32 - protein, 98 aa, synthetic construct
  REGION 1..98: note = ENPP121GLK Protein Export Signal Sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLK

SEQ ID NO: 33 - protein, 623 aa, synthetic construct
  REGION 1..623: note = Albumin Sequence
GGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 34 - protein, 227 aa, synthetic construct
  REGION 1..227: note = Human IgG Fc domain, Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 618 aa, synthetic construct
  REGION 1..618: note = Albumin Sequence
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 36 - protein, 16 aa, synthetic construct
  REGION 1..16: note = ENPP2 Signal Peptide
LFTFAVGVNICLGFTA

SEQ ID NO: 37 - protein, 20 aa, synthetic construct
  REGION 1..20: note = Signal Sequence ENPP7
MRGPAVLLTVALATLLAPGA

SEQ ID NO: 38 - protein, 22 aa, synthetic construct
  REGION 1..22: note = Signal sequence ENPP7
MRGPAVLLTVALATLLAPGAGA

SEQ ID NO: 39 - protein, 95 aa, synthetic construct
  REGION 1..95: note = Signal Sequence ENPP1-2-1
MERDGCAGGGSRGGEGGRAPREGPAGNGRDRGRSHAAEAPGDPQAAASLLAPMDVGEEPLEKAARARTAKDPNTYKIISLFTFAVGVNICLGFTA

SEQ ID NO: 40 - protein, 17 aa, synthetic construct
  REGION 1..17: note = exENPP3
LLVIMSLGLGLGLGLRK

SEQ ID NO: 41 - protein, 22 aa, synthetic construct
  REGION 1..22: note = Signal Sequence ENPP5
MTSKFLLVSFILAALSLSTTFS

SEQ ID NO: 42 - protein, 19 aa, synthetic construct
  REGION 1..19: note = Signal Sequence - Azurocidin
MTRLTVLALLAGLLASSRA

SEQ ID NO: 43-46 - skipped (4 entries)

SEQ ID NO: 47 - protein, 15 aa, synthetic construct
  REGION 1..15: note = Linker
  VARIANT 1..15: note = Features section of the listing that any one or all of amino acids 1-15 can either be present or absent
RRRRRRRRRRRRRRR

SEQ ID NO: 48 - protein, 16 aa, synthetic construct
  REGION 1..16: note = Linker
DSSSEEKFLRRIGRFG

SEQ ID NO: 49 - protein, 12 aa, synthetic construct
  REGION 1..12: note = Linker
EEEEEEEPRGDT

SEQ ID NO: 50 - protein, 12 aa, synthetic construct
  REGION 1..12: note = Linker
APWHLSSQYSRT

SEQ ID NO: 51 - protein, 12 aa, synthetic construct
  REGION 1..12: note = Linker
STLPIPHEFSRE

SEQ ID NO: 52 - protein, 12 aa, synthetic construct
  REGION 1..12: note = Linker
VTKHLNQISQSY

SEQ ID NO: 53 - protein, 15 aa, synthetic construct
  REGION 1..15: note = Linker
  VARIANT 2..15: note = Features section of the listing that any one or all of amino acids 2-15 can either be present or absent
EEEEEEEEEEEEEEE

SEQ ID NO: 54 - skipped

SEQ ID NO: 55 - protein, 6 aa, synthetic construct
  REGION 1..6: note = Linker
GGSGGS

SEQ ID NO: 56 - protein, 7 aa, synthetic construct
  REGION 1..7: note = Linker
RSGSGGS

SEQ ID NO: 57 - protein, 15 aa, synthetic construct
  REGION 1..15: note = Linker
  VARIANT 2..15: note = Features section of the listing that any one or all of amino acids 2-15 can either be present or absent
DDDDDDDDDDDDDDD

SEQ ID NO: 58 - protein, 16 aa, synthetic construct
  REGION 1..16: note = Linker
LVIMSLGLGLGLGLRK

SEQ ID NO: 59 - protein, 15 aa, synthetic construct
  REGION 1..15: note = Linker
VIMSLGLGLGLGLRK

SEQ ID NO: 60 - protein, 14 aa, synthetic construct
  REGION 1..14: note = Linker
IMSLGLGLGLGLRK

SEQ ID NO: 61 - protein, 13 aa, synthetic construct
  REGION 1..13: note = Linker
MSLGLGLGLGLRK

SEQ ID NO: 62 - protein, 12 aa, synthetic construct
  REGION 1..12: note = Linker
SLGLGLGLGLRK

SEQ ID NO: 63 - protein, 11 aa, synthetic construct
  REGION 1..11: note = Linker
LGLGLGLGLRK

SEQ ID NO: 64 - protein, 10 aa, synthetic construct
  REGION 1..10: note = Linker
GLGLGLGLRK

SEQ ID NO: 65 - protein, 9 aa, synthetic construct
  REGION 1..9: note = Linker
LGLGLGLRK

SEQ ID NO: 66 - protein, 8 aa, synthetic construct
  REGION 1..8: note = Linker
GLGLGLRK

SEQ ID NO: 67 - protein, 7 aa, synthetic construct
  REGION 1..7: note = Linker
LGLGLRK

SEQ ID NO: 68 - protein, 6 aa, synthetic construct
  REGION 1..6: note = Linker
GLGLRK

SEQ ID NO: 69 - protein, 5 aa, synthetic construct
  REGION 1..5: note = Linker
LGLRK

SEQ ID NO: 70 - protein, 4 aa, synthetic construct
  REGION 1..4: note = Linker
GLRK

SEQ ID NO: 71-72 - skipped (2 entries)

SEQ ID NO: 73 - protein, 15 aa, synthetic construct
  REGION 1..15: note = Linker
  VARIANT 1..15: note = Features section of the listing that any one or all of amino acids 1-15 can either be present or absent
KKKKKKKKKKKKKKK

SEQ ID NO: 74 - protein, 15 aa, synthetic construct
  REGION 1..15: note = Linker
  VARIANT 2..15: note = Features section of the listing that any one or all of amino acids 2-15 can either be present or absent
DDDDDDDDDDDDDDD

SEQ ID NO: 75 - protein, 50 aa, synthetic construct
  REGION 1..50: note = Linker
  VARIANT 6..50: note = Amino acids 6-50 can either be present or absent in groups of 5(GGGGS) starting at position 6
GGGGSGGGGSGGGGSGGGGSGGGGSGGGGSGGGGSGGGGSGGGGSGGGGS

SEQ ID NO: 76 - DNA, 2625 bp, synthetic construct
  misc_feature 1..2625: note = ENPP3 Nucleotide sequence
  source 1..2625: mol_type = other DNA
atggaatctacgttgactttagcaacggaacaacctgttaagaagaacactcttaagaaatataaaatagcttgcattgttcttcttgctttgctggtgatcatgtcacttggattaggcctggggcttggactcaggaaactggaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactatt

SEQ ID NO: 77 - DNA, 2775 bp, synthetic construct
  misc_feature 1..2775: note = ENPP1 Nucleotide sequence
  source 1..2775: mol_type = other DNA
atggaacgggacggctgtgccggcggaggatcaagaggcggagaaggcggcagagcccctagagaaggacctgccggcaacggcagagacagaggcagatctcatgccgccgaagcccctggcgatcctcaggctgctgcttctctgctggcccccatggatgtgggcgaggaacctctggaaaaggccgccagagccagaaccgccaaggaccccaacacctacaaggtgctgagcctggtgctgtccgtgtgcgtgctgaccaccatcctgggctgcatcttcggcctgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagat

SEQ ID NO: 78 - DNA, 3255 bp, synthetic construct
  misc_feature 1..3255: note = Azurocidin-ENPP1-FC Nucleotide sequence
  source 1..3255: mol_type = other DNA
ggtaccgccaccatgacaagactgacagtgctggctctgctggccggactgttggcctcttctagagctgctccttcctgcgccaaagaagtgaagtcctgcaagggcagatgcttcgagcggaccttcggcaactgtagatgtgacgccgcttgcgtggaactgggcaactgctgcctggactaccaagagacatgcatcgagcccgagcacatctggacctgcaacaagttcagatgcggcgagaagcggctgaccagatctctgtgcgcctgctctgacgactgcaaggacaagggcgactgctgcatcaactactcctctgtgtgccagggcgagaagtcctgggttgaagaaccctgcgagtccatcaacgagcctcagtgtcctgccggcttcgagacacctcctactctgctgttctccctggatggcttcagagccgagtacctgcatacttggggaggcctgctgccagtgatctccaagctgaagaagtgcggcacctacaccaagaacatgaggcctgtgtaccctaccaagacattccccaaccactactccatcgtgaccggcctgtatcctgagagccacggcatcatcgacaacaagatgtacgaccccaagatgaacgcctccttcagcctgaagtccaaagagaagttcaaccccgagtggtataagggcgagcctatctgggtcaccgctaagtaccagggactgaagtctggcaccttcttttggcctggctccgacgtggaaatcaacggcatcttccccgacatctataagatgtacaacggctccgtgcctttcgaggaacgcattctggctgttctgcagtggctgcagctgcctaaggatgagaggcctcacttctacaccctgtacctggaagaacctgactcctccggccactcttatggccctgtgtcctctgaagtgatcaaggccctgcagcgagtggacggaatggtcggaatgctgatggacggcctgaaagagctgaacctgcacagatgcctgaacctgatcctgatctccgaccacggcatggaacaggggagctgcaagaagtacatctacctgaacaagtacctgggcgacgtgaagaacatcaaagtgatctacggcccagccgccagactgaggccttctgatgtgcctgacaagtactactccttcaactacgagggaatcgcccggaacctgtcctgcagagagcctaaccagcacttcaagccctacctgaagcactttctgcctaagcggctgcacttcgccaagtctgacagaatcgagcccctgaccttctatctggaccctcagtggcagctggccctgaatcctagcgagagaaagtactgtggctccggcttccacggctccgacaacgtgttctctaatatgcaggccctgttcgtcggctacggccctggctttaaacacggcatcgaggccgacaccttcgagaacatcgaggtgtacaatctgatgtgtgacctgctgaatctgacccctgctcctaacaacggcacccacggatctctgaaccatctgctgaagaatcccgtgtacacccctaagcaccccaaagaggttcaccctctggtccagtgtcctttcaccagaaatcctcgggacaacctgggctgctcttgcaacccttctatcctgcctatcgaggactttcagacccagttcaacctgaccgtggccgaggaaaagatcatcaagcacgagacactgccctacggcagacctagagtgctgcagaaagagaacaccatctgcctgctgtcccagcaccagttcatgtccggctactcccaggacatcctgatgcctctgtggacctcctacaccgtggaccggaacgatagcttctccaccgaggacttcagcaactgcctgtaccaggatttcagaatccctctgagccccgtgcacaagtgcagcttctacaagaacaacaccaaggtgtcctacggcttcctgtctcctccacagctgaacaagaactccagcggcatctactctgaggccctgctgaccaccaacatcgtgcccatgtaccagtccttccaagtgatctggcggtacttccacgacaccctgctgaggaagtacgccgaagaaagaaacggcgtgaacgtggtgtctggccccgtgttcgacttcgactacgacggcagatgcgactctctggaaaacctgcggcagaaaagacgagtgatccggaatcaagagatcctgattcctacacacttctttatcgtgctgaccagctgcaaggatacctctcagacccctctgcactgcgagaatctggacaccctggccttcattctgcctcacagaaccgacaactccgagtcctgtgtgcacggcaagcacgactcctcttgggtcgaagaactgctgatgctgcaccgggccagaatcaccgatgtggaacacatcaccggcctgagcttctaccagcagcggaaagaacctgtgtccgatatcctgaagctgaaaacccatctgccaaccttcagccaagaggacctgatcaacgacaagacccacacctgtcctccatgtcctgctccagaactgctcggaggcccctctgtgttcctgtttccacctaagccaaaggacacactgatgatctctcggacccctgaagtgacctgcgtggtggtggatgtgtctcacgaagatcccgaagtcaagttcaattggtacgtggacggcgtggaagtgcacaacgccaagaccaagcctagagaggaacagtacaactccacctacagagtggtgtccgtgctgactgtgctgcaccaggattggctgaacggcaaagagtacaagtgcaaagtgtccaacaaggctctgcccgctcctatcgaaaagaccatctccaaggctaagggccagcctcgggaacctcaggtttacaccctgcctccatctcgggaagagatgaccaagaaccaggtgtccctgacctgcctggtcaagggcttctacccttccgatatcgccgtggaatgggagtccaatggccagcctgagaacaactacaagacaacccctcctgtgctggacagcgacggctcattcttcctgtactctaagctgacagtggacaagtcccggtggcagcaaggcaatgtgttttcctgctctgtgatgcacgaggccctccacaatcactacacccagaagtccctgtctctgtcccctggcaaatgatagctcgag

SEQ ID NO: 79 - DNA, 3219 bp, synthetic construct
  misc_feature 1..3219: note = Azurocidin-ENPP3-FC Nucleotide sequence
  source 1..3219: mol_type = other DNA
atgaccagactgaccgtgctggccctgctggccggcctgctggccagcagcagagccgccaagcagggcagctgcagaaagaagtgcttcgacgccagcttcagaggcctggagaactgcagatgcgacgtggcctgcaaggacagaggcgactgctgctgggacttcgaggacacctgcgtggagagcaccagaatctggatgtgcaacaagttcagatgcggcgagaccagactggaggccagcctgtgcagctgcagcgacgactgcctgcagagaaaggactgctgcgccgactacaagagcgtgtgccagggcgagaccagctggctggaggagaactgcgacaccgcccagcagagccagtgccccgagggcttcgacctgccccccgtgatcctgttcagcatggacggcttcagagccgagtacctgtacacctgggacaccctgatgcccaacatcaacaagctgaagacctgcggcatccacagcaagtacatgagagccatgtaccccaccaagaccttccccaaccactacaccatcgtgaccggcctgtaccccgagagccacggcatcatcgacaacaacatgtacgacgtgaacctgaacaagaacttcagcctgagcagcaaggagcagaacaaccccgcctggtggcacggccagcccatgaacctgaccgccatgtaccagggcctgaaggccgccacctacttctggcccggcagcgaggtggccatcaacggcagcttccccagcatctacatgccctacaacggcagcgtgcccttcgaggagagaatcagcaccctgctgaagtggctggacctgcccaaggccgagagacccagattctacaccatgtacttcgaggagcccgacagcagcggccacgccggcggccccgtgagcgccagagtgatcaaggccctgcaggtggtggaccacgccttcggcatgctgatggagggcctgaagcagagaaacctgcacaactgcgtgaacatcatcctgctggccgaccacggcatggaccagacctactgcaacaagatggagtacatgaccgactacttccccagaatcaacttcttctacatgtacgagggccccgcccccagaatcagagcccacaacatcccccacgacttcttcagcttcaacagcgaggagatcgtgagaaacctgagctgcagaaagcccgaccagcacttcaagccctacctgacccccgacctgcccaagagactgcactacgccaagaacgtgagaatcgacaaggtgcacctgttcgtggaccagcagtggctggccgtgagaagcaagagcaacaccaactgcggcggcggcaaccacggctacaacaacgagttcagaagcatggaggccatcttcctggcccacggccccagcttcaaggagaagaccgaggtggagcccttcgagaacatcgaggtgtacaacctgatgtgcgacctgctgagaatccagcccgcccccaacaacggcacccacggcagcctgaaccacctgctgaaggtgcccttctacgagcccagccacgccgaggaggtgagcaagttcagcgtgtgcggcttcgccaaccccctgcccaccgagagcctggactgcttctgcccccacctgcagaacagcacccagctggagcaggtgaaccagatgctgaacctgacccaggaggagatcaccgccaccgtgaaggtgaacctgcccttcggcagacccagagtgctgcagaagaacgtggaccactgcctgctgtaccacagagagtacgtgagcggcttcggcaaggccatgagaatgcccatgtggagcagctacaccgtgccccagctgggcgacaccagccccctgccccccaccgtgcccgactgcctgagagccgacgtgagagtgccccccagcgagagccagaagtgcagcttctacctggccgacaagaacatcacccacggcttcctgtacccccccgccagcaacagaaccagcgacagccagtacgacgccctgatcaccagcaacctggtgcccatgtacgaggagttcagaaagatgtgggactacttccacagcgtgctgctgatcaagcacgccaccgagagaaacggcgtgaacgtggtgagcggccccatcttcgactacaactacgacggccacttcgacgcccccgacgagatcaccaagcacctggccaacaccgacgtgcccatccccacccactacttcgtggtgctgaccagctgcaagaacaagagccacacccccgagaactgccccggctggctggacgtgctgcccttcatcatcccccacagacccaccaacgtggagagctgccccgagggcaagcccgaggccctgtgggtggaggagagattcaccgcccacatcgccagagtgagagacgtggagctgctgaccggcctggacttctaccaggacaaggtgcagcccgtgagcgagatcctgcagctgaagacctacctgcccaccttcgagaccaccatcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

(19) United States
(12) Patent Application Publication
(10) Pub. No.: US 2023/0372455 A1
(43) Pub. Date: Nov. 23, 2023

(54) COMPOSITIONS AND METHODS FOR TREATING PERIPHERAL ARTERY DISEASE

(71) Applicants: Inozyme Pharma, Inc., Boston, MA (US); Westfälische Wilhelms-Universität Münster, Münster (DE)

(72) Inventors: David Thompson, Boston, MA (US); Frank Rutsch, Münster (DE); Yvonne Nitschke, Münster (DE)

(21) Appl. No.: 18/058,715

(22) Filed: Nov. 23, 2022

Related U.S. Application Data
(63) Continuation of application No. PCT/US21/34533, filed on May 27, 2021.
(60) Provisional application No. 63/030,840, filed on May 27, 2020.

(51) Int. Cl. A61K 38/46; A61P 9/10; A61K 38/38; A61L 31/08; A61L 31/16

(52) U.S. Cl. CPC A61K 38/46; A61P 9/10; A61K 38/38; C12Y 306/01009; A61L 31/08; A61L 31/16; C07K 2319/30; A61L 2300/416; A61L 2300/254; A61L 2300/252

(57) ABSTRACT

The present disclosure provides compositions and methods for treating Peripheral Artery Diseases in a subject by administering an ectonucleotide pyrophosphatase phosphodiesterase-1 (ENPP1) agent or an ectonucleotide pyrophosphatase phosphodiesterase-3 (ENPP3).

### RELATED APPLICATIONS

This application is a continuation of International Patent Application No. PCT/US2021/034533, filed May 27, 2021, which claims priority to U.S. Application No. 63/030,840 filed on May 27, 2020, the content of each is herein incorporated by reference in its entirety.

### FIELD OF THE DISCLOSURE

The disclosure relates to compositions and methods of treating vascular diseases.

### SEQUENCE LISTING

This application contains a Sequence Listing which has been submitted electronically as a WIPO Standard ST.26 XML file via Patent Center, created on Jul. 21, 2023, is entitled “4427-10204.xml” and is 161,138 bytes in size. The sequence listing is incorporated herein by reference in its entirety.

### BACKGROUND

Peripheral Artery Disease (PAD) is a common disorder that occurs due to atherosclerosis characterized by stenosis and/or obstruction of lower limbs arteries leading to decreased muscle perfusion and oxygenation. Symptomatic PAD patients suffer symptoms of intermittent claudication (IC), defined as fatigue, discomfort, or pain occurring in limb muscles during effort, due to exercise-induced ischemia. PAD is progressive and can lead to necrosis, gangrene, and need for limb amputation. Due to the complexity and multifactorial origins of PAD, as well as the differences in muscular adaptive responses, precise PAD pathophysiological mechanisms are still largely unknown.

### SUMMARY OF THE DISCLOSURE

The disclosure is based, at least in part, on the surprising discovery that an ENPP1 polypeptide inhibited the unwanted proliferation of vascular smooth muscle cells that occurs following trauma to peripheral arteries in mammals. As described in the working examples, the therapeutic effects of an ENPP1-Fc fusion protein were assessed with respect to the ability to inhibit stenosis after angioplasty in previously injured and stented peripheral arteries of Yorkshire swine. Animals treated with the ENPP1-Fc protein exhibited markedly lower intimal thickening in stented profunda arteries relative to animals treated with a vehicle control, demonstrating that ENPP1 has therapeutic utility in inhibiting and/or preventing intimal thickening and/or proliferation in injured peripheral arteries.

Accordingly, in one aspect, the disclosure relates to a method for treating a subject having peripheral artery disease, the method comprising: administering to the subject an effective amount of an ENPP1 agent to thereby treat said peripheral artery disease in said subject.

The disclosure includes a method for reducing and/or preventing progression of vascular smooth muscle cell proliferation in a peripheral artery of a subject, the method comprising: administering to the subject an effective amount of an ENPP1 agent to thereby reduce and/or prevent progression of said vascular smooth muscle cell proliferation in said peripheral artery of said subject.

In some embodiments of any of the methods described herein, the subject has stage III, stage IV or stage IV, grade III peripheral artery disease.

The disclosure also includes a method for inhibiting or slowing progression of Stage III peripheral artery disease to Stage IV peripheral artery disease in a subject, the method comprising: administering to the subject an effective amount of an ENPP1 agent to thereby inhibit and/or slow progression of Stage III peripheral artery disease to Stage IV peripheral artery disease in said subject.

In some embodiments of any of the methods described herein, the subject has common femoral artery disease.

In some embodiments of any of the methods described herein, the subject has femoral-popliteal disease.

In some embodiments of any of the methods described herein, the subject has tibial-peroneal disease.

The disclosure also relates to a method for reducing and/or preventing progression of vascular smooth muscle cell proliferation in a peripheral artery of a subject who undergoes surgery on said peripheral artery, the method comprising: administering to the subject an effective amount of an ENPP1 agent to thereby reduce and/or prevent progression of vascular smooth muscle cell proliferation in said peripheral artery at a surgical site of said peripheral artery in said subject.

In some embodiments of any of the methods described herein, the agent is administered prior to, during and/or after said surgery.

In some embodiments of any of the methods described herein, the surgery comprises placement of a stent.

In some embodiments of any of the methods described herein, the ENPP1 agent comprises an ENPP1 polypeptide.

In some embodiments of any of the methods described herein, the ENPP1 agent comprises ENPP1 variants that retain enzymatic activity.

In some embodiments of any of the methods described herein, the ENPP1 agent comprises a nucleic acid encoding an ENPP1 polypeptide.

In some embodiments of any of the methods described herein, the ENPP1 agent comprises a viral vector comprising a nucleic acid encoding an ENPP1 polypeptide.

In some embodiments of any of the methods described herein, the ENPP1 polypeptide comprises the extracellular domain of ENPP1.

In some embodiments of any of the methods described herein, the ENPP1 polypeptide comprises the catalytic domain of ENPP1.

In some embodiments of any of the methods described herein, the ENPP1 polypeptide comprises amino acids 99 to 925 of SEQ ID NO:1.

In some embodiments of any of the methods described herein, the ENPP1 polypeptide comprises a heterologous protein.

In some embodiments of any of the methods described herein, the heterologous protein increases the circulating half-life of the ENPP1 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP1 polypeptide.

In some embodiments of any of the methods described herein, ENPP1 agent comprises a linker.

In some embodiments of any of the methods described herein, the linker separates the ENPP1 polypeptide and the heterologous protein.

In some embodiments of any of the methods described herein, the linker comprises the following amino acid sequence: (GGGGS)ₙ, wherein n is an integer from 1 to 10.

In some embodiments of any of the methods described herein, the ENPP1 agent is administered to the subject subcutaneously.

In some embodiments of any of the methods described herein, the ENPP1 agent is administered to the subject intravenously.

In some embodiments of any of the methods described herein, the subject: is a tobacco user, has hypertension, has elevated cholesterol or triglyceride levels, is a diabetic, has renal disease, or is obese.

In another aspect, the disclosure also includes a method for reducing and/or preventing progression of vascular smooth muscle cell proliferation in a peripheral artery of a subject who undergoes stent placement in said peripheral artery, the method comprising: administering to the subject an effective amount of an ENPP1 agent to thereby reduce and/or prevent progression of vascular smooth muscle cell proliferation in said peripheral artery.

In another aspect, the disclosure features a method for reducing and/or preventing stenosis or restenosis in a peripheral artery of a subject who undergoes stent placement in said peripheral artery, the method comprising: administering to the subject an effective amount of an ENPP1 agent to thereby reduce and/or prevent stenosis or restenosis in said peripheral artery.

In some embodiments of any of the methods described herein, the agent is administered prior to, during and/or after stent placement.

In another aspect, the disclosure features a method for treating a subject having peripheral artery disease, the method comprising: administering to the subject an effective amount of an ENPP3 agent to thereby treat said peripheral artery disease in said subject.

In yet another aspect, the disclosure features a method for reducing and/or preventing progression of vascular smooth muscle cell proliferation in a peripheral artery of a subject having peripheral artery disease, the method comprising: administering to the subject an effective amount of an ENPP3 agent to thereby reduce and/or prevent progression of said vascular smooth muscle cell proliferation in said peripheral artery of said subject.

In some embodiments of any of the methods described herein, the subject has stage III, stage IV or stage IV, grade III peripheral artery disease.

In another aspect, the disclosure features a method for inhibiting or slowing progression of Stage III peripheral artery disease to Stage IV peripheral artery disease in a subject, the method comprising: administering to the subject an effective amount of an ENPP3 agent to thereby inhibit and/or slow progression of Stage III peripheral artery disease to Stage IV peripheral artery disease in said subject.

In some embodiments of any of the methods described herein, the subject has common femoral artery disease.

In some embodiments of any of the methods described herein, the subject has femoral-popliteal disease.

In some embodiments of any of the methods described herein, the subject has tibial-peroneal disease.

In another aspect, the disclosure features a method for reducing and/or preventing progression of vascular smooth muscle cell proliferation in a peripheral artery of a subject who requires surgery on said peripheral artery, wherein the subject has peripheral artery disease, the method comprising: administering to the subject an effective amount of an ENPP3 agent to thereby reduce and/or prevent progression of vascular smooth muscle cell proliferation in said peripheral artery at a surgical site of said peripheral artery in said subject.

In some embodiments of any of the methods described herein, the agent is administered prior to, during and/or after said surgery.

In some embodiments of any of the methods described herein, the surgery comprises placement of a stent.

In some embodiments of any of the methods described herein, the subject does not have a deficiency of ENPP1.

In some embodiments of any of the methods described herein, the ENPP3 agent comprises an ENPP3 polypeptide.

In some embodiments of any of the methods described herein, the ENPP3 agent comprises ENPP3 variants that retain enzymatic activity.

In some embodiments of any of the methods described herein, the ENPP3 agent comprises a nucleic acid encoding an ENPP3 polypeptide.

In some embodiments of any of the methods described herein, the ENPP3 agent comprises a viral vector comprising a nucleic acid encoding an ENPP3 polypeptide.

In some embodiments of any of the methods described herein, the ENPP3 polypeptide comprises the extracellular domain of ENPP3.

In some embodiments of any of the methods described herein, the ENPP3 polypeptide comprises the catalytic domain of ENPP3.

In some embodiments of any of the methods described herein, the ENPP3 polypeptide comprises amino acids 49 to 875 of SEQ ID NO:7

In some embodiments of any of the methods described herein, the ENPP3 polypeptide comprises a heterologous protein.

In some embodiments of any of the methods described herein, the heterologous protein increases the circulating half-life of the ENPP3 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP3 polypeptide.

In some embodiments of any of the methods described herein, the ENPP3 agent comprises a linker.

In some embodiments of any of the methods described herein, the linker separates the ENPP3 polypeptide and the heterologous protein.

In some embodiments of any of the methods described herein, the linker comprises the following amino acid sequence: (GGGGS)n, wherein n is an integer from 1 to 10.

In some embodiments of any of the methods described herein, the ENPP3 agent is administered to the subject subcutaneously.

In some embodiments of any of the methods described herein, the ENPP3 agent is administered to the subject intravenously.

In some embodiments of any of the methods described herein, the subject: is a tobacco user, has hypertension, has elevated cholesterol or triglyceride levels, is a diabetic, has renal disease, or is obese.

In some embodiments of any of the methods described herein, the subject has occlusive peripheral artery disease.

In another aspect, the disclosure features a method for reducing and/or preventing progression of vascular smooth muscle cell proliferation in a peripheral artery of a subject who undergoes stent placement in said peripheral artery, the method comprising: administering to the subject an effective amount of an ENPP3 agent to thereby reduce and/or prevent progression of vascular smooth muscle cell proliferation in said peripheral artery.

In another aspect, the disclosure features a method for reducing and/or preventing stenosis or restenosis in a peripheral artery of a subject who undergoes stent placement in said peripheral artery, the method comprising: administering to the subject an effective amount of an ENPP3 agent to thereby reduce and/or prevent stenosis or restenosis in said peripheral artery.

In some embodiments of any of the methods described herein, the ENPP3 agent is administered prior to, during and/or after stent placement.

In yet another aspect, the disclosure features a coated stent comprising a vascular stent; and a coating on the stent, the coating comprising an ENPP1 agent; and a carrier for said ENPP1 agent, wherein said coating is configured to release said ENPP1 agent from the stent at a rate of 1-10 μg/ml per day.

In some embodiments of any of the stents described herein, the ENPP1 agent in an amount between 1 wt % and 50 wt %, based on a total weight of the coating.

In some embodiments of any of the stents described herein, the ENPP1 agent is selected from a group consisting of: ENPP1, ENPP1-Fc, ENPP1-Albumin, and ENPP1 mRNA.

In some embodiments of any of the stents described herein, the ENPP1 agent comprises ENPP1 variants that retain enzymatic activity.

In some embodiments of any of the stents described herein, the carrier is non-reactive with said ENPP1 agent.

In some embodiments of any of the stents described herein, the carrier comprises a polymeric carrier that is physically bound to said ENPP1 agent.

In some embodiments of any of the stents described herein, the carrier comprises a polymeric carrier that is chemically bound to said ENPP1 agent.

In some embodiments of any of the stents described herein, the carrier comprises a polymeric biodegradable carrier.

In some embodiments of any of the stents described herein, the carrier comprises a nonpolymeric carrier.

In some embodiments of any of the stents described herein, the nonpolymeric carrier is selected from a group consisting of: Vitamin E, Vitamin E acetate, Vitamin E succinate, oleic acid, peanut oil and cottonseed oil.

In some embodiments of any of the stents described herein, the carrier is a liquid at body temperature.

In some embodiments of any of the stents described herein, the carrier is a solid at body temperature.

In yet another aspect, the disclosure features a coated stent comprising a vascular stent; and a coating on the stent, the coating comprising an ENPP3 agent; and a carrier for said ENPP3 agent, wherein said coating is configured to release said ENPP3 agent from the stent at a rate of 1-10 μg/ml per day.

In some embodiments of any of the stents described herein, the ENPP3 agent in an amount between 1 wt % and 50 wt %, based on a total weight of the coating.

In some embodiments of any of the stents described herein, the ENPP3 agent is selected from a group consisting of: ENPP3, ENPP3-Fc, ENPP3-Albumin, and ENPP3 mRNA.

In some embodiments of any of the stents described herein, the ENPP3 agent comprises ENPP3 variants that retain enzymatic activity.

In some embodiments of any of the stents described herein, the carrier is non-reactive with said ENPP3 agent.

In some embodiments of any of the stents described herein, the carrier comprises a polymeric carrier that is physically bound to said ENPP3 agent.

In some embodiments of any of the stents described herein, the carrier comprises a polymeric carrier that is chemically bound to said ENPP3 agent.

In some embodiments of any of the stents described herein, the carrier comprises a polymeric biodegradable carrier.

In some embodiments of any of the stents described herein, the carrier comprises a nonpolymeric carrier.

In some embodiments of any of the stents described herein, the nonpolymeric carrier is selected from a group consisting of: Vitamin E, Vitamin E acetate, Vitamin E succinate, oleic acid, peanut oil and cottonseed oil.

In some embodiments of any of the stents described herein, the carrier is liquid at body temperature.

In some embodiments of any of the stents described herein, the carrier is solid at body temperature.

In yet another aspect, the disclosure features a method for treating a subject having peripheral artery disease, the method comprising: implanting an arterial stent coated with an ENPP1 agent into an artery of said subject, wherein said implanted stent is configured to release said ENPP1 agent in an amount effective to thereby treat said peripheral artery disease in said subject.

In yet another aspect, the disclosure features a method for reducing and/or preventing progression of vascular smooth muscle cell proliferation in a peripheral artery of a subject having peripheral artery disease, the method comprising: implanting an arterial stent coated with an ENPP1 agent into an artery of said subject, wherein said implanted stent is configured to release said ENPP1 agent in an amount effective to thereby reduce and/or prevent progression of said vascular smooth muscle cell proliferation in said peripheral artery of said subject.

In some embodiments of any of the methods described herein, the subject has stage III, stage IV or stage IV, grade III peripheral artery disease.

In yet another aspect, the disclosure features a method for inhibiting or slowing progression of Stage III peripheral artery disease to Stage IV peripheral artery disease in a subject, the method comprising: implanting an arterial stent coated with an ENPP1 agent into an artery of said subject, wherein said implanted stent is configured to release said ENPP1 agent in an amount effective to thereby inhibit and/or slow progression of Stage III peripheral artery disease to Stage IV peripheral artery disease in said subject.

In some embodiments of any of the methods described herein, the subject has common femoral artery disease.

In some embodiments of any of the methods described herein, the subject has femoral-popliteal disease.

In some embodiments of any of the methods described herein, the subject has tibial-peroneal disease.

In yet another aspect, the disclosure features a method for reducing and/or preventing progression of vascular smooth muscle cell proliferation in a peripheral artery of a subject who has a condition requiring surgery on said peripheral artery, wherein the subject has peripheral artery disease, the method comprising: implanting an arterial stent coated with an ENPP1 agent into an artery of said subject, wherein said implanted stent is configured to release said ENPP1 agent in an amount effective to thereby reduce and/or prevent progression of vascular smooth muscle cell proliferation in said peripheral artery at a surgical site of said peripheral artery in said subject.

In some embodiments of any of the methods described herein, the agent is administered prior to, during and/or after said surgery.

In some embodiments of any of the methods described herein, the condition requiring surgery is due to a prior placement of a non-eluting arterial stent in said artery.

In some embodiments of any of the methods described herein, the condition requiring is due to a prior placement of an eluting arterial stent in said artery which elutes therapeutic agents other than said ENPP1 agent.

In some embodiments of any of the methods described herein, the subject does not have a deficiency of ENPP1.

In some embodiments of any of the methods described herein, the ENPP1 agent comprises an ENPP1 polypeptide.

In some embodiments of any of the methods described herein, the ENPP1 agent comprises a nucleic acid encoding an ENPP1 polypeptide.

In some embodiments of any of the methods described herein, the ENPP1 agent comprises a viral vector comprising a nucleic acid encoding an ENPP1 polypeptide.

In some embodiments of any of the methods described herein, the ENPP1 polypeptide comprises the extracellular domain of ENPP1.

In some embodiments of any of the methods described herein, the ENPP1 polypeptide comprises the catalytic domain of ENPP1.

In some embodiments of any of the methods described herein, the ENPP1 polypeptide comprises amino acids 99 to 925 of SEQ ID NO:1.

In some embodiments of any of the methods described herein, the ENPP1 polypeptide comprises a heterologous protein.

In some embodiments of any of the methods described herein, the heterologous protein increases the circulating half-life of the ENPP1 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP1 polypeptide.

In some embodiments of any of the methods described herein, the ENPP1 agent comprises a linker.

In some embodiments of any of the methods described herein, the ENPP1 polypeptide and the heterologous protein.

In some embodiments of any of the methods described herein, the linker comprises the following amino acid sequence: (GGGGS)n, wherein n is an integer from 1 to 10.

In some embodiments of any of the methods described herein, the ENPP1 agent is administered to the subject subcutaneously.

In some embodiments of any of the methods described herein, wherein the ENPP1 agent is administered to the subject intravenously.

In some embodiments of any of the methods described herein, the subject: is a tobacco user, has hypertension, has elevated cholesterol or triglyceride levels, is a diabetic, has renal disease, or is obese.

In yet another aspect, the disclosure features a method for treating a subject having peripheral artery disease, the method comprising: implanting an arterial stent coated with an ENPP3 agent into an artery of said subject, wherein said implanted stent is configured to release said ENPP3 agent in an amount effective to thereby treat said peripheral artery disease in said subject.

In yet another aspect, the disclosure features a method for reducing and/or preventing progression of vascular smooth muscle cell proliferation in a peripheral artery of a subject having peripheral artery disease, the method comprising: implanting an arterial stent coated with an ENPP3 agent into an artery of said subject, wherein said implanted stent is configured to release said ENPP3 agent in an amount effective to thereby reduce and/or prevent progression of said vascular smooth muscle cell proliferation in said peripheral artery of said subject.

In some embodiments of any of the methods described herein, the subject has stage III, stage IV or stage IV, grade III peripheral artery disease.

In yet another aspect, the disclosure features a method for inhibiting or slowing progression of Stage III peripheral artery disease to Stage IV peripheral artery disease in a subject, the method comprising: implanting an arterial stent coated with an ENPP3 agent into an artery of said subject, wherein said implanted stent is configured to release said ENPP1 agent in an amount effective to thereby inhibit and/or slow progression of Stage III peripheral artery disease to Stage IV peripheral artery disease in said subject.

In some embodiments of any of the methods described herein, the subject has common femoral artery disease.

In some embodiments of any of the methods described herein, the subject has femoral-popliteal disease.

In some embodiments of any of the methods described herein, the subject has tibial-peroneal disease.

In yet another aspect, the disclosure features a method for reducing and/or preventing progression of vascular smooth muscle cell proliferation in a peripheral artery of a subject who has a condition requiring surgery on said peripheral artery, wherein the subject has peripheral artery disease, the method comprising: implanting an arterial stent coated with an ENPP3 agent into an artery of said subject, wherein said implanted stent is configured to release said ENPP3 agent in an amount effective to thereby reduce and/or prevent progression of vascular smooth muscle cell proliferation in said peripheral artery at a surgical site of said peripheral artery in said subject.

In some embodiments of any of the methods described herein, the agent is administered prior to, during and/or after said surgery.

In some embodiments of any of the methods described herein, the condition requiring surgery is due to a prior placement of a non-eluting arterial stent in said artery.

In some embodiments of any of the methods described herein, the condition requiring is due to a prior placement of an eluting arterial stent in said artery which elutes therapeutic agents other than said ENPP3 agent.

In some embodiments of any of the methods described herein, the subject does not have a deficiency of ENPP1.

In some embodiments of any of the methods described herein, the ENPP1 agent comprises an ENPP3 polypeptide.

In some embodiments of any of the methods described herein, the ENPP3 agent comprises a nucleic acid encoding an ENPP1 polypeptide.

In some embodiments of any of the methods described herein, the ENPP3 agent comprises a viral vector comprising a nucleic acid encoding an ENPP3 polypeptide.

In some embodiments of any of the methods described herein, the ENPP3 polypeptide comprises the extracellular domain of ENPP3.

In some embodiments of any of the methods described herein, the ENPP3 polypeptide comprises the catalytic domain of ENPP3.

In some embodiments of any of the methods described herein, the ENPP3 polypeptide comprises amino acids 49-875 of SEQ ID NO:7.

In some embodiments of any of the methods described herein, the ENPP3 polypeptide comprises a heterologous protein.

In some embodiments of any of the methods described herein, the heterologous protein increases the circulating half-life of the ENPP3 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP3 polypeptide.

In some embodiments of any of the methods described herein, the ENPP3 agent comprises a linker.

In some embodiments of any of the methods described herein, the linker separates the ENPP3 polypeptide and the heterologous protein.

In some embodiments of any of the methods described herein, the linker comprises the following amino acid sequence: (GGGGS)n, wherein n is an integer from 1 to 10.

In some embodiments of any of the methods described herein, the ENPP3 agent is administered to the subject subcutaneously.

In some embodiments of any of the methods described herein, the ENPP3 agent is administered to the subject intravenously.

In some embodiments of any of the methods described herein, the subject: is a tobacco user, has hypertension, has elevated cholesterol or triglyceride levels, is a diabetic, has renal disease, or is obese.

Other features and advantages of the disclosure will be apparent from the following detailed description and claims.

### BRIEF DESCRIPTION OF THE DRAWINGS

FIG. 1 is a series of photographs of representative profunda artery images captured by angiography at day 14 and day 42 post stent implantation. The two control images illustrate a narrowing of the profunda due to intimal proliferation at day 42 relative to the morphology of the vessel at day 14. By contrast, in animals treated with ENPP1-Fc little visible change in profunda morphology was observed between day 14 and day 42. The upper and lower boundary of the stent within the vessel is identified in each photograph by rectangles.

FIG. 2 is a series of photographs of representative profunda artery images captured by Optical Coherence Tomography (OCT) at day 14 and day 42 post stent implantation. The two control images illustrate a pronounced intimal thickening within the profunda at day 42 relative to the morphology of the vessel at day 14. By contrast, in animals treated with ENPP1-Fc little visible intimal thickening was observed between day 14 and day 42. The extent of stenosis is highlighted in the day 42 photographs.

FIG. 3 is a bar graph depicting the percent area of stenosis at day 14 and day 42 in the profunda of pigs treated with ENPP1-Fc (Treatment) or given vehicle control (Control), as measured by OCT.

FIG. 4A is a cross-section of an artery experiencing restenosis in the presence of an uncoated stent. The endothelium 12 normally serves as a solid barrier between the layer of smooth muscle cells 14 and the arterial lumen 20. Small tears 16 in the endothelium 12 can expose smooth muscle cells 14, which can then migrate into the arterial lumen 20 and hyper proliferate into a mass 18 which can partially or completely occlude the lumen 20 even though an uncoated stent 21 is placed, during a procedure 60 such as angioplasty, in the artery 10 to keep the arterial lumen 20 open.

FIG. 4B is a cross-section of an artery 10 containing a coated stent 22. The stent has a coating 24 containing a carrier and a bioactive compound such as ENPP1 agent 65 that inhibits and or prevents restenosis. By using a stent having this coating 24, the tears 16 shown in FIG. 4A in the endothelium 12 may be reduced or eliminated. Additionally, the mass 18 created by a proliferation of smooth muscle cells 14, as shown in FIG. 4A, is eliminated or substantially reduced.

### DETAILED DESCRIPTION

### Definitions

Unless defined otherwise, all technical and scientific terms used herein have the same meaning as commonly understood by one of ordinary skill in the art to which this disclosure belongs. Although any methods and materials similar or equivalent to those described herein can be used in practice or testing of the present disclosure, the preferred methods and materials are described.

For clarity, “NPP1” and “ENPP1” refer to the same protein and are used interchangeably herein. As used herein, the term “ENPP1 protein” or “ENPP1 polypeptide” refers to ectonucleotide pyrophosphatase/phosphodiesterase-1 protein encoded by the ENPP1 gene that is capable of cleaving ATP to generate PPi and also reduces ectopic calcification in soft tissue.

ENPP1 protein is a type II transmembrane glycoprotein and cleaves a variety of substrates, including phosphodiester bonds of nucleotides and nucleotide sugars and pyrophosphate bonds of nucleotides and nucleotide sugars. ENPP1 protein has a transmembrane domain and soluble extracellular domain. The extracellular domain is further subdivided into somatomedin B domain, catalytic domain and the nuclease domain. The sequence and structure of wild-type ENPP1 is described in detail in PCT Application Publication No. WO 2014/126965 to Braddock, et al., which is incorporated herein in its entirety by reference.

ENPP1 polypeptides as used herein encompasses polypeptides that exhibit ENPP1 enzymatic activity, mutants of ENPP1 that retain ENPP1 enzymatic activity, fragments of ENPP1 or variants of ENPP1 including deletion variants that exhibit ENPP1 enzymatic activity. ENPP1 enzymatic activity refers to the ability of the ENPP1 polypeptide to cleave Adenosine Triphosphate (ATP) into plasma pyrophosphate (PPi), as noted below.

ENPP3 polypeptides as used herein encompasses polypeptides that exhibit ATP cleavage enzymatic activity, mutants of ENPP3 that retain ATP cleavage enzymatic activity, fragments of ENPP3 or variants of ENPP3 including deletion variants that exhibit ATP cleavage enzymatic activity. ATP cleavage enzymatic activity refers to the ability of the ENPP3 polypeptide to cleave Adenosine Triphosphate (ATP) into plasma pyrophosphate (PPi), as noted below.

Some examples of ENPP1 and ENPP3 polypeptides, mutants, or mutant fragments thereof, have been previously disclosed in International PCT Application Publications No. WO/2014/126965—Braddock et al., WO/2016/187408-Braddock et al., WO/2017/087936-Braddock et al., and WO2018/027024-Braddock et al., all of which are incorporated by reference in their entireties herein.

Enzymatically active” with respect to an ENPP1 polypeptide or an ENPP3 polypeptide is defined as possessing ATP hydrolytic activity into AMP and PPi and/or AP3a hydrolysis to ADP and AMP. NPP1 and NPP3 readily hydrolyze ATP into AMP and PPi. The steady-state Michaelis-Menten enzymatic constants of NPP1 are determined using ATP as a substrate. NPP1 can be demonstrated to cleave ATP by HPLC analysis of the enzymatic reaction, and the identity of the substrates and products of the reaction are confirmed by using ATP, AMP, and ADP standards. The ATP substrate degrades over time in the presence of NPP1, with the accumulation of the enzymatic product AMP. Using varying concentrations of ATP substrate, the initial rate velocities for NPP1 are derived in the presence of ATP, and the data is fit to a curve to derive the enzymatic rate constants. At physiologic pH, the kinetic rate constants of NPP1 are Km=144 μM and kcat=7.8 s⁻¹.

As used herein the term “plasma pyrophosphate (PPi) levels” refers to the amount of pyrophosphate present in plasma of animals. In certain embodiments, animals include rat, mouse, cat, dog, human, cow and horse. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications.

Typically, plasma PPi levels in healthy human subjects range from about 1 μm to about 3 μM, in some cases between 1-2 μm. Subjects who have defective ENPP1 expression tend to exhibit low ppi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with Generalized Arterial Calcification of Infancy (GACI), the ppi levels are found to be less than 1 μm and in some cases are below the level of detection. In patients afflicted with Pseudoxanthoma Elasticum (PXE), the ppi levels are below 0.5 μm. (Arterioscler Thromb Vasc Biol. 2014 September; 34(9): 1985-9; Braddock et al., Nat Commun. 2015; 6: 10006.)

As used herein, the term “PPi” refers to inorganic pyrophosphate.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

As used herein, the term “ENPP1 precursor protein” refers to ENPP1 with its signal peptide sequence at the ENPP1 N-terminus. Upon proteolysis, the signal sequence is cleaved from ENPP1 to provide the ENPP1 protein. Signal peptide sequences useful within the disclosure include, but are not limited to, Albumin signal sequence, Azurocidin signal sequence, ENPP1 signal peptide sequence, ENPP2 signal peptide sequence, ENPP7 signal peptide sequence, and/or ENPPS signal peptide sequence.

As used herein, the term “ENPP3 precursor protein” refers to ENPP3 with its signal peptide sequence at the ENPP3 N-terminus. Upon proteolysis, the signal sequence is cleaved from ENPP3 to provide the ENPP3 protein. Signal peptide sequences useful within the disclosure include, but are not limited to, Albumin signal peptide sequence, Azurocidin signal peptide sequence, ENPP1 signal peptide sequence, ENPP2 signal peptide sequence, ENPP7 signal peptide sequence, and/or ENPP5 signal peptide sequence.

As used herein, the term “Azurocidin signal peptide sequence” refers to the signal peptide derived from human azurocidin. Azurocidin, also known as cationic antimicrobial protein CAP37 or heparin-binding protein (HBP), is a protein that in humans is encoded by the AZU1 gene. The nucleotide sequence encoding Azurocin signal peptide MTRLTVLALLAGLLASSRA (SEQ ID NO: 42) is fused with the nucleotide sequence of NPP1 or NPP3 gene which when encoded generates ENPP1 precursor protein or ENPP3 precursor protein. (Optimized signal peptides for the development of high expressing CHO cell lines, Kober et al., Biotechnol Bioeng. 2013 April; 110(4): 1164-73)

As used herein, the term “ENPP1-Fc construct” refers to ENPP1 (e.g., the extracellular domain of ENPP1) recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “ENPP3-Fc construct” refers to ENPP3 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “Fc” refers to a human IgG (immunoglobulin) Fc domain. Subtypes of IgG such as IgG1, IgG2, IgG3, and IgG4 are contemplated for use as Fc domains. The “Fc region or Fc polypeptide” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein the term “functional equivalent variant”, as used herein, relates to a polypeptide substantially homologous to the sequences of ENPP1 or ENPP3 (defined above) and that preserves the enzymatic and biological activities of ENPP1 or ENPP3, respectively. Methods for determining whether a variant preserves the biological activity of the native ENPP1 or ENPP3 are widely known to the skilled person and include any of the assays used in the experimental part of said application. Particularly, functionally equivalent variants of ENPP1 or ENPP3 delivered by viral vectors is encompassed by the present disclosure.

The functionally equivalent variants of ENPP1 or ENPP3 are polypeptides substantially homologous to the native ENPP1 or ENPP3 respectively. The expression “substantially homologous”, relates to a protein sequence when said protein sequence has a degree of identity with respect to the ENPP1 or ENPP3 sequences described above of at least 80%, at least 85%, at least 90%, at least 91%, at least 92%, at least 93%, at least 94%, at least 95%, at least 96%, at least 97%, at least 98% or at least 99% respectively and still retaining at least 50%, 55%, 60%, 70%, 80% or 90% activity of wild type ENPP1 or ENPP3 protein with respect to ATP cleavage.

The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)), though other similar algorithms can also be used. BLAST and BLAST 2.0 are used, with the parameters described herein, to determine percent sequence identity. Software for performing BLAST analyses is publicly available through the National Center for Biotechnology Information.

“Functionally equivalent variants” of ENPP1 or ENPP3 may be obtained by replacing nucleotides within the polynucleotide accounting for codon preference in the host cell that is to be used to produce the ENPP1 or ENPP3 respectively. Such “codon optimization” can be determined via computer algorithms which incorporate codon frequency tables such as “Human high.cod” for codon preference as provided by the University of Wisconsin Package Version 9.0, Genetics Computer Group, Madison, Wis. The variants of ENPP1 or ENPP3 polypeptides are expected to retain at least 50%, 55%, 60%, 70%, 80% or 90% activity of wild type ENPP1 or ENPP3 protein with respect to ATP cleavage.

As used herein, the term “wild-type” refers to a gene or gene product isolated from a naturally occurring source. A wild-type gene is most frequently observed in a population and is thus arbitrarily designed the “normal” or “wild-type” form of the human NPP1 or NPP3 genes. In contrast, the term “functionally equivalent” refers to a NPP1 or NPP3 gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As defined herein, the term “moiety” refers to a chemical component or biological molecule that can be covalently or non-covalently linked to ENPP1 or ENPP3 polypeptide and has the ability to confer a desired property to the protein to which it is attached. For example, the term moiety can refer to a bone targeting peptide such as polyaspartic acid or polyglutamic acid (of 4-20 consecutive asp or glu residues) or a molecule that extends the half-life of ENPP1 or ENPP3 polypeptide. Some other examples of moieties include Fc, albumin, transferrin, polyethylene glycol (PEG), homo-amino acid polymer (HAP), proline-alanine-serine polymer (PAS), elastin-like peptide (ELP), and gelatin-like protein (GLK).

As defined herein, the term “subject”, “individual” or “patient” refers to mammal preferably a human who does not possess a loss of function mutation in the NPP1 gene, such as those loss of function mutations that result in pathological calcification and pathological ossification diseases such as Generalized Arterial Calcification of Infancy (GACI), Autosomal Recessive Hypophosphatemic Rickets Type 2 (ARHR2), Infantile idiopathic arterial calcification (IIAC), Ossification of the Posterior Longitudinal Ligament (OPLL), hypophosphatemic rickets, osteoarthritis, calcification of atherosclerotic plaques, hereditary and non-hereditary forms of osteoarthritis, ankylosing spondylitis, hardening of the arteries occurring with aging, calciphylaxis resulting from end stage renal disease and progeria. Such a patient will have a normal level of NPP1 in serum which refers to the amount of NPP1 required to maintain a normal level of plasma pyrophosphate (PPi) in a healthy subject. A normal level of PPi corresponds to 2-3 μM.

As defined herein, the phrase “medial area” is the area between lamina elastica externa and lamina elastica interna of an artery.

As defined herein, the phrase “intimal area” and said intimal area is the area between said lamina elastica interna and lumen of an artery.

As defined herein, the phrase “lamina elastica externa” refers to a layer of elastic connective tissue lying immediately outside the smooth muscle of the tunica media of an artery.

As defined herein, the phrase “lamina elastica interna” refers to a layer of elastic tissue that forms the outermost part of the tunica intima of blood vessels.

As defined herein, the phrase “lumen” refers to the interior of a vessel, such as the central space in an artery, vein or capillary through which blood flow occurs.

As defined herein, the phrase “surgery” refers to an invasive medical procedure that involves vascular interventions which result in tissue injury by scapel incision or radiofrequency ablation or cryoablation or laser ablation.

As defined herein, the phrase “tissue injury” refers to proliferation or onset of proliferation and migration of vascular smooth muscle eventually resulting in the thickening of arterial walls and decreased arterial lumen space resulting restenosis after percutaneous vascular interventions such as stenting or angioplasty.

As defined herein, the phrase “deficient for NPP1” or “ENPP1 deficiency” refers to a reduction in an amount of NPP1 protein or in NPP1 activity relative to a normal serum level of NPP1 protein or normal activity of NPP1, wherein such a reduction results in a disease or disorder of pathological calcification and/or pathological ossification. Such pathological diseases include but are not limited to GACI and ARHR2. ENPP1 deficiency, as used herein, does not refer to small reductions in an amount of NPP1 protein and/or NPP1 activity that do not result in a disease or disorder of pathological calcification and/or pathological ossification.

As defined herein, the phrase “vascular trauma” refers to injury to a blood vessel—an artery, which carries blood to an extremity or an organ, or a vein, which returns blood to the heart. Vascular injuries may also be caused by invasive procedures, such as vascular bypass surgery.

As defined herein the phrase “accidental trauma” refers to a blood vessel such as artery by a blunt injury which occurs when a blood vessel is crushed or stretched due to exertion of physical force or penetrating injury which occurs when a blood vessel is punctured, torn or severed. Blunt injury occurs during physical alterations such as boxing and penetrating injury occurs due to sharp objects such as knife or bullet wounds. The trauma or injury can be caused by different factors, such as radiation, viral infections, development of immune complexes, and hyperlipidemia.

As defined herein the phrase “restenosis” refers to recurrence of stenosis. Stenosis refers to the narrowing of a blood vessel, leading to restricted blood flow. Restenosis usually pertains to an artery or other large blood vessel that has become narrowed, received treatment to clear the blockage and subsequently become re-narrowed. Restenosis is commonly detected by using one or more of ultrasound, X-ray computed tomography (CT), nuclear imaging, optical imaging or contrast enhanced image or immunohistochemical detection.

As defined herein the phrase “myointimal proliferation” refers to the proliferation of vascular smooth muscle cells that occurs at the tunica intima of an arterial wall of an individual.

As used herein the term “ENPP1 fragment” refers to a fragment or a portion of ENPP1 protein or an active subsequence of the full-length NPP1 having at least an ENPP1 catalytic domain administered in protein form or in the form of a nucleic acid encoding the same.

As used herein, the term “ENPP1 agent” refers to ENPP1 polypeptide or fusion protein or ENPP1 fragment comprising at least catalytic domain capable of producing plasma pyrophosphate (Ppi) by cleavage of adenosine triphosphate (ATP) or a polynucleotide such as cDNA or RNA encoding ENPP1 fusion protein or ENPP1 fragment comprising at least catalytic domain capable of producing PPi by enzymatic cleavage of ATP or a vector such as a viral vector containing a polynucleotide encoding the same.

As used herein the term “ablation” refers to the removal or destruction of a body part or tissue or its function. Ablation may be performed by surgery, hormones, drugs, radiofrequency, heat.

As used herein, the phrase “reduce or prevent myointimal proliferation” refers to the ability of soluble NPP1 upon administration to reduce the level of proliferation vascular smooth muscle cells at the site of tissue injury thereby reducing the thickening of arterial walls and prevent the occurrence of or reduce the level of restenosis of the artery.

As used herein the term “non-surgical tissue injury” refers to injuries sustained to a tissue or blood vessel during a traumatic event including but not limited to physical altercations involving use of blunt force or sharp objects such as knife, mechanical injury such fall from elevation, workplace injury due to heavy machinery or vehicular injury such as car accidents.

As used herein, the term “stent” refers to a tubular support placed inside a blood vessel, canal, or duct to aid healing or relieve an obstruction or prevent narrowing of the passage. Stents generally comprise an expandable mesh coil which is made of metal (ex: stainless steel, Cobalt alloy, Nickel-titanium alloy, manganese alloy, molybdenum alloy, platinum alloy, tungsten alloy) or polymers (ex: Silicone).

As used herein, the term “vascular stent” refers to a tubular support placed inside an artery or vein of a mammal to aid healing or relieve an obstruction or prevent narrowing of the arterial passage.

As used herein, the term “coated stent” or “eluting stent” refers to a stent that is coated with a therapeutic molecule such as protein, chemical compound or nucleic acid that gradually elutes from the stent surface (interior or exterior) at the site of implantation thereby providing therapeutic relief. Therapeutic molecules such as ENPP1 agent or ENPP3 agent can be bonded directly to a metal stent, and some are bonded to a matrix polymer, which acts as a drug reservoir to ensure drug retention during deployment and a uniform distribution on the stent. The types, compositions, and designs of the polymers coated on the stent dictate the eluting kinetic of the sustain time release of the drug over a period of weeks or months following the implantation in situ. The coating materials can be categorized as organic vs inorganic, bioerodable vs nonbioerodable, and synthetic vs naturally occurring substances.

As used herein, the term “coating” refers to composition comprising a polymeric carrier that is used in conjunction with an ENPP1 agent or ENPP3 agent to coat the stents. The coating may be applied in the form a spray or dried film comprising the ENPP1 agent or ENPP3 agent suspended in a polymeric matrix. The polymeric carrier is in an amount sufficient to provide a polymer matrix or support for the ENPP1 agent or ENPP3 agent. The polymer is preferably non-reactive with the ENPP1 agent or ENPP3 agent, i.e., no chemical reaction occurs when the two are mixed.

As used herein, the term “solvent” is defined according to its broadest recognized definition and includes any material into which the carrier (polymer) and the ENPP1 agent or ENPP3 agent can dissolve, fully or partially, at room temperature or from 20° C. to 40° C. to form the coating composition. Sterile, double distilled water is a preferred solvent.

As used herein, the term “site of injury” refers to a region in the vasculature where the flow of blood or spinal fluid is constricted due to accumulation of one or more of lipids, cholesterol, calcium, and various types of cells, such as smooth muscle cells and platelets. The site of injury is commonly identified by using Cardiac catheterization. During a cardiac catheterization, a long, narrow tube called a catheter is inserted through a plastic introducer sheath (a short, hollow tube that is inserted into a blood vessel in your arm or leg). The catheter is guided through the blood vessel to the coronary arteries with the aid of an x-ray machine. Contrast material is injected through the catheter and x-ray images (Coronary angiogram) are created as the contrast material moves through the heart's chambers, valves and major vessels. The digital photographs of the contrast material are used to identify the site of the narrowing or blockage in the coronary artery. Additional imaging procedures, called intra-vascular ultrasound (NUS) and fractional flow reserve (FFR), may be performed along with cardiac catheterization in some cases to obtain detailed images of the walls of the blood vessels.

As used herein “site of implant” refers to the region at which the ENPP1 or ENPP3 coated stent is implanted in the vasculature. The coated stents of the invention can be placed at the center of the to the site of tissue injury, immediately adjacent the site of tissue injury or within 200 μm on either side from the center of the site of tissue injury.

As used herein, the term “myocardial infarction” refers to a permanent damage to the heart muscle that occurs due to the formation of plaques in the interior walls of the arteries resulting in reduced blood flow to the heart and injuring heart muscles because of lack of oxygen supply. The symptoms of MI include chest pain, which travels from left arm to neck, shortness of breath, sweating, nausea, vomiting, abnormal heart beating, anxiety, fatigue, weakness, stress, depression, and other factors.

As used herein the term “blunt force trauma” refers to a physical trauma to a body part, either by impact, injury or physical attack or high-velocity impact. Blunt trauma can lead to contusions, abrasions, lacerations, and/or bone fractures.

As used herein the term “non-surgical tissue injury” or “penetrating trauma” refers to trauma to a body part which occurs when an object such as a projectile or knife enters a tissue of the body, creating an open wound.

As used herein the term “scapel incision” refers to incision made in a tissue using a sharp object such as a scapel during surgical procedure. An incision is a cut made into the tissues of the body to expose the underlying tissue, bone, or organ so that a surgical procedure can be performed.

As used herein the term “Peripheral artery disease” (PAD) refers to the narrowing of the peripheral arteries serving the legs, stomach, arms and head. (“Peripheral” in this case means away from the heart, in the outer regions of the body.) PAD most commonly affects arteries in the legs. PAD generally occurs due to atherosclerosis, a buildup of cholesterol and fatty deposits (plaque) which narrows or blocks blood flow to the arteries leading to the arms, legs and feet. The supply of oxygen to cells is also limited due to the plaque buildup in the artery walls. The most common symptoms of PAD involving the lower extremities are cramping, pain or tiredness in the leg or hip muscles while walking or climbing stairs. Patients with peripheral arterial disease have a higher risk of coronary artery disease, heart attack or stroke. Left untreated, PAD can lead to gangrene and amputation. The American College of Cardiology/American Heart Association Practice Guidelines defines the presentation of PAD by four categories: asymptomatic, claudication, critical limb ischemia, and acute limb ischemia (ALI). There are at least two major classification criterion that are routine used in art to classify the level of severity of PAD, Fontaine Classification system and Rutherford Classification system (Overview of Classification Systems in Peripheral Artery Disease, Rulon L. Hardman, Semin Intervent Radiol 2014; 31: 378-388)

As used herein the term “Claudication” refers to fatigue, discomfort, or pain in the lower extremities, typically the calves, which is reproducibly brought on by exercise and relieved by rest.

As used herein the term “Critical Limb Ischemia (CLI)” refers to a condition wherein the patient experiences chronic ischemic rest pain, nocturnal recumbent pain, or ischemic skin lesions that may include ulcers or gangrene.

As used herein the term” Acute Limb Ischemia (ALI)” refers to patients with a sudden decrease in limb perfusion causing an immediate threat to limb viability.

As used herein the term “Fontaine Classification System” refers to classification system developed by Fontaine et al. (Fontaine R, Kim M, Kieny R. Surgical treatment of peripheral circulation disorders [in German]. Helv Chir Acta 1954; 21(5-6):499-533). This classification system grades the clinical presentation of patients to four stages. The system is solely based on clinical symptoms, without other diagnostic tests and is as shown in table below.

| GRADE | SYMPTOMS |
|---|---|
| Stage I PAD | Asymptomatic, incomplete blood vessel |
| | obstruction |
| Stage II PAD | Mild claudication pain in limb |
| Stage IIA PAD | Claudication at a distance >200 m |
| Stage IIB PAD | Claudication at a distance <200 m |
| Stage III PAD | Rest pain, mostly in the feet |
| Stage IV PAD | Necrosis and/or gangrene of the limb |

As used herein the term “Rutherford Classification System” refers to classification system developed by Rutherford et al. (Rutherford R B, Flanigan D P, Gupta S K, et al. Suggested standards for reports dealing with lower extremity ischemia. J Vasc Surg 1986; 4(1):80-94). The Rutherford system classifies PAD into acute and chronic limb ischemia, emphasizing that each presentation requires different treatment algorithms. The Rutherford classification also associates patient clinical symptoms with objective findings, including Doppler, arterial brachial indices (ABI), and pulse volume recordings.

Rutherford's chronic limb ischemia classification most resembles Fontaine's classification, with the addition of objective noninvasive data such as treadmill test. Treadmill protocols are well described in other publications. (Hoyer C, Sandermann J, Petersen Li The toe-brachial index in the diagnosis of peripheral arterial disease. J Vasc Surg 2013; 58(1): 231-238). Treadmill exercise testing with and without preexercise and postexercise ABIs helps differentiate claudication from pseudoclaudication in patients with exertional leg symptoms. Patients who cannot perform treadmill testing can undergo similar stress testing using plantar flexion or thigh blood pressure cuff compression to cause reactive hyperemia. Rutherford's classification for chronic limb ischemia is shown in table below.

| | | CLINICAL | OBJECTIVE |
|---|---|---|---|
| GRADE | CATEGORY | DESCRIPTION | CRITERIA |
| 0 | 0 | Asymptomatic-no | Normal treadmill or |
| | | hemodynamically | reactive hyperemia test |
| | | significant occlusive | |
| | | disease | |
| | 1 | Mild Claudication | Completes treadmill |
| | | | exercise; AP after |
| | | | exercise >50 mm Hg but |
| | | | at least 20 mm Hg lower |
| | | | than resting value |
| I | 2 | Moderate Claudication | Between categories 1 and 3 |
| | 3 | Severe Claudication | Cannot complete standard |
| | | | treadmill exercise, and AP |
| | | | after exercise <50 mm Hg |
| II | 4 | Ischemic Rest Pain | Resting AP <40 mm Hg, |
| | | | flat or barely pulsatile ankle |
| | | | or metatarsal PVR; TP <30 |
| | | | mm Hg |
| III | 5 | Minor tissue loss- | Resting AP < 60 mm Hg, |
| | | nonhealing ulcer, focal | ankle or metatarsal PVR |
| | | gangrene with diffuse | flat or barely pulsatile; |
| | | pedal ischemia | TP <40 mm Hg |
| | 6 | Major tissue loss- | Same as category 5 |
| | | extending above TM | |
| | | level, functional foot no | |
| | | longer salvageable | |

Abbreviations: AP, ankle pressure; PVR, pulse volume recording; TM, transmetatarsal; TP, toe pressure.

As used herein the term “toe pressure” refers to the measurement of the blood pressure in the toe compared to the blood pressure in the arm

As used herein the term “ankle pressure” refers to the measurement of the blood pressure in the lower leg compared to the blood pressure in the arm

As used herein the term “pulse volume recording” refers to a noninvasive vascular test in which blood pressure cuffs and a hand-held ultrasound device (such as a Doppler or transducer) are used to obtain information about arterial blood flow in the arms and legs. As used herein, the term “stage III peripheral artery disease” refers to the PAD disease stage as indicated by the Fontaine classification system

As used herein, the term “stage IV peripheral artery disease” refers to a PAD disease stage as classified by the Fontaine classification system

As used herein, the term “stage IV, grade III peripheral artery disease” refers to a PAD disease stage as classified by the Rutherford classification system

As used herein, the term “common femoral artery disease” refers to a disease state wherein occlusion due to PAD occurs in the femoral artery of the patient.

As used herein, the term “femoral-popliteal disease” refers to a disease state wherein patients have obstructive occlusions due to PAD in the femoropopliteal artery.

As used herein, the term “tibial-peroneal disease” refers to a disease state wherein patients have obstructive occlusions due to PAD in a segment of the artery, referred to as tibial-peroneal trunk, below the knee, distal to the origin of the anterior tibial artery off the popliteal artery, and proximal to the branch point of the posterior tibial artery and the fibular artery.

As used herein, the term “subject who requires surgery” refers to a patient who is not ENPP1 deficient and has arterial occlusion in the peripheral arteries such as femoral, femoropopliteal or tibial-peroneal arteries.

As used herein, the term “site of surgery” refers to the region of the artery upon which a tissue injury has occurred either due to vascular trauma or accidental trauma.

As used herein the term “angioplasty” refers to a medical procedure that opens up a blocked or narrowed artery around the heart. It is a standard treatment for narrowed or blocked arteries

A “low level of PPi” refers to a condition in which the subject has at least 0.1%-0.99% less than 2%-5% of normal levels of plasma pyrophosphate (PPi). Normal levels of Plasma PPi in healthy human subjects are in the range of 1.8 to 2.6 μM. +/−0.1 μM (Arthritis and Rheumatism, Vol. 22, No. 8 (August 1979))

As used herein, the term “treatment” or “treating” is defined as the application or administration of soluble NPP1 (alone or in combination with another pharmaceutical agent), to a patient, or application or administration of a therapeutic agent to an isolated tissue or cell line from a patient (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

As used herein, the term “prevent” or “prevention” or “reduce” means no disorder or disease development if none had occurred, or no further disorder or disease development if there had already been development of the disorder or disease. Also considered is the ability of one to prevent some or all of the symptoms associated with the disorder or disease.

As used herein, the term “effective amount” refers to an amount of an agent (e.g., NPP1 fusion or NPP3 fusion polypeptides) which, as compared to a corresponding subject who has not received such an amount, sufficient to provide improvement of a condition, disorder, disease, or to provide a decrease in progression or advancement of a condition, disorder, or disease. An effective amount also may result in treating, healing, preventing or ameliorating a condition, disease, or disorder. The term also includes within its scope amounts effective to enhance normal physiological function. As used herein, the term “polypeptide” refers to a polymer composed of amino acid residues, related naturally occurring structural variants, and synthetic non-naturally occurring analogs thereof linked via peptide bonds.

As used here the term “Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein the term “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained; for example, phosphate-buffered saline (PBS)

As used herein, the term “pathological calcification” refers to the abnormal deposition of calcium salts in blood vessels, soft tissues, secretory and excretory passages of the body causing it to harden. There are two types, dystrophic calcification which occurs in dying and dead tissue and metastatic calcification which elevated extracellular levels of calcium (hypercalcemia), exceeding the homeostatic capacity of cells and tissues. Calcification can involve cells as well as extracellular matrix components such as collagen in basement membranes and elastic fibers in arterial walls. Some examples of tissues prone to calcification include: Gastric mucosa—the inner epithelial lining of the stomach, Kidneys and lungs, Cornea, heart valves, Systemic arteries and Pulmonary veins.

As used herein, the term “pathological ossification” refers to a pathological condition in which bone arises in tissues not in the osseous system and in connective tissues usually not manifesting osteogenic properties. Ossification is classified into three types depending on the nature of the tissue or organ being affected, endochondral ossification is ossification that occurs in and replaces cartilage. Intramembranous ossification is ossification of bone that occurs in and replaces connective tissue. Metaplastic ossification the development of bony substance in normally soft body structures; called also heterotrophic ossification.

As used herein, “reduction of calcification” is observed by using non-invasive methods like X-rays, micro CT and MRI. Reduction of calcification is also inferred by using radio imaging with 99mTc-pyrophosphate (99mPYP) uptake. The presence of calcifications in mice are evaluated via post-mortem by micro-computed tomography (CT) scans and histologic sections taken from the heart, aorta and kidneys with the use of dyes such as Hematoxylin and Eosin (H&E) and Alizarin red by following protocols established by Braddock et al. (Nature Communications volume 6, Article number: 10006 (2015))

As used herein the term “Ectopic calcification” refers to a condition characterized by a pathologic deposition of calcium salts in tissues or bone growth in soft tissues.

As used herein the term “Ectopic calcification of soft tissue” refers to inappropriate biomineralization, typically composed of calcium phosphate, hydroxyapatite, calcium oxalates and ocatacalcium phosphates occurring in soft tissues leading to loss of hardening of soft tissues. “Arterial calcification” refers to ectopic calcification that occurs in arteries and heart valves leading to hardening and or narrowing of arteries. Calcification in arteries is correlated with atherosclerotic plaque burden and increased risk of myocardial infarction, increased ischemic episodes in peripheral vascular disease, and increased risk of dissection following angioplasty.

As used herein, the term “Venous calcification” refers to ectopic calcification that occurs in veins that reduces the elasticity of the veins and restricts blood flow which can then lead to increase in blood pressure and coronary defects.

As used herein, the term “Vascular calcification” refers to the pathological deposition of mineral in the vascular system. It has a variety of forms, including intimal calcification and medial calcification, but can also be found in the valves of the heart. Vascular calcification is associated with atherosclerosis, diabetes, certain heredity conditions, and kidney disease, especially CKD. Patients with vascular calcification are at higher risk for adverse cardiovascular events. Vascular calcification affects a wide variety of patients. Idiopathic infantile arterial calcification is a rare form of vascular calcification where the arteries of neonates calcify.

As used herein, the term “Brain calcification” (BC) refers to a nonspecific neuropathology wherein deposition of calcium and other mineral in blood vessel walls and tissue parenchyma occurs leading to neuronal death and gliosis. Brain calcification is” often associated with various chronic and acute brain disorders including Down's syndrome, Lewy body disease, Alzheimer's disease, Parkinson's disease, vascular dementia, brain tumors, and various endocrinologic conditions Calcification of heart tissue refers to accumulation of deposits of calcium (possibly including other minerals) in tissues of the heart, such as aorta tissue and coronary tissue.

The terms “adeno-associated viral vector”, “AAV vector”, “adeno-associated virus”, “AAV virus”, “AAV virion”, “AAV viral particle” and “AAV particle”, as used interchangeably herein, refer to a viral particle composed of at least one AAV capsid protein (preferably by all of the capsid proteins of a particular AAV serotype) and an encapsidated recombinant viral genome. The particle comprises a recombinant viral genome having a heterologous polynucleotide comprising a sequence encoding human ENPP1 or human ENPP3 or a functionally equivalent variant thereof) and a transcriptional regulatory region that at least comprises a promoter flanked by the AAV inverted terminal repeats. The particle is typically referred to as an “AAV vector particle” or “AAV vector”.

As used herein, the term “vector” means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional nucleotide sequences may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) is integrated into the genome of a host cell upon introduction into the host cell, and thereby are replicated along with the host genome. Moreover, certain vectors (expression vectors) are capable of directing the expression of genes to which they are operatively linked.

As used herein, the term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “recombinant viral genome”, as used herein, refers to an AAV genome in which at least one extraneous expression cassette polynucleotide is inserted into the naturally occurring AAV genome. The genome of the AAV according to the disclosure typically comprises the cis-acting 5′ and 3′ inverted terminal repeat sequences (ITRs) and an expression cassette.

The term “expression cassette”, as used herein, refers to a nucleic acid construct, generated recombinantly or synthetically, with a series of specified nucleic acid elements, which permit transcription of a particular nucleic acid in a target cell. The expression cassette of the recombinant viral genome of the AAV vector according to the disclosure comprises a transcriptional regulatory region operatively linked to a nucleotide sequence encoding ENPP1 or ENPP3 or a functionally equivalent variant thereof.

The term “transcriptional regulatory region”, as used herein, refers to a nucleic acid fragment capable of regulating the expression of one or more genes. The transcriptional regulatory region according to the disclosure includes a promoter and, optionally, an enhancer.

The term “promoter”, as used herein, refers to a nucleic acid fragment that functions to control the transcription of one or more polynucleotides, located upstream the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. Any kind of promoters may be used in the disclosure including inducible promoters, constitutive promoters and tissue-specific promoters.

The term “enhancer”, as used herein, refers to a DNA sequence element to which transcription factors bind to increase gene transcription. Examples of enhancers may be, without limitation, RSV enhancer, CMV enhancer, HCR enhancer, etc. In another embodiment, the enhancer is a liver-specific enhancer, more preferably a hepatic control region enhancer (HCR).

The term “operatively linked”, as used herein, refers to the functional relation and location of a promoter sequence with respect to a polynucleotide of interest (e.g. a promoter or enhancer is operably linked to a coding sequence if it affects the transcription of the sequence). Generally, a promoter operatively linked is contiguous to the sequence of interest. However, an enhancer does not have to be contiguous to the sequence of interest to control its expression. In another embodiment, the promoter and the nucleotide sequence encoding ENPP1 or ENPP3 or a functionally equivalent variant thereof.

The term “effective amount” refers to a nontoxic but sufficient amount of a viral vector encoding ENPP1 or ENPP3 to provide the desired biological result. That result may be reduction and/or alleviation of the signs, symptoms, or causes of a disease, or any other desired alteration of a biological system.

The term “Cap protein”, as used herein, refers to a polypeptide having at least one functional activity of a native AAV Cap protein (e.g. VP1, VP2, VP3). Examples of functional activities of Cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present disclosure.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “Rep protein”, as used herein, refers to a polypeptide having at least one functional activity of a native AAV Rep protein (e.g. Rep 40, 52, 68, 78). A “functional activity” of a Rep protein is any activity associated with the physiological function of the protein, including facilitating replication of DNA through recognition, binding and nicking of the AAV origin of DNA replication as well as DNA helicase activity.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. Procedures for modifying these ITR sequences are known in the art (Brown T, “Gene Cloning”, Chapman & Hall, London, G B, 1995; Watson R, et al., “Recombinant DNA”, 2^{nd} Ed. Scientific American Books, New York, N.Y., US, 1992; Alberts B, et al., “Molecular Biology of the Cell”, Garland Publishing Inc., New York, N.Y., US, 2008; Innis M, et al., Eds., “PCR Protocols. A Guide to Methods and Applications”, Academic Press Inc., San Diego, Calif., US, 1990; and Schleef M, Ed., “Plasmid for Therapy and Vaccination”, Wiley-VCH Verlag GmbH, Weinheim, Del., 2001).

The term “tissue-specific” promoter is only active in specific types of differentiated cells or tissues. Typically, the downstream gene in a tissue-specific promoter is one which is active to a much higher degree in the tissue(s) for which it is specific than in any other. In this case there may be little or substantially no activity of the promoter in any tissue other than the one(s) for which it is specific.

The term “inducible promoter”, as used herein, refers to a promoter that is physiologically or developmentally regulated, e.g. by the application of a chemical inducer. For example, it can be a tetracycline-inducible promoter, a mifepristone (RU-486)-inducible promoter and the like.

The term “constitutive promoter”, as used herein, refers to a promoter whose activity is maintained at a relatively constant level in all cells of an organism, or during most developmental stages, with little or no regard to cell environmental conditions. In another embodiment, the transcriptional regulatory region allows constitutive expression of ENPP1. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter (Boshart M, et al., Cell 1985; 41:521-530).

The term “polyadenylation signal”, as used herein, relates to a nucleic acid sequence that mediates the attachment of a polyadenine stretch to the 3′ terminus of the mRNA. Suitable polyadenylation signals include, without limitation, the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like.

The term “signal peptide”, as used herein, refers to a sequence of amino acid residues (ranging in length from 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish et al., 2000, Molecular Cell Biology, 4th edition).

As used herein, the term “immune response” or “immune reaction” refers to the host's immune system to antigen in an invading (infecting) pathogenic organism, or to introduction or expression of foreign protein. The immune response is generally humoral and local; antibodies produced by B cells combine with antigen in an antigen-antibody complex to inactivate or neutralize antigen. Immune response is often observed when human proteins are injected into mouse model systems. Generally, the mouse model system is made immune tolerant by injecting immune suppressors prior to the introduction of a foreign antigen to ensure better viability.

As used herein, the term “immunesuppression” is a deliberate reduction of the activation or efficacy of the host immune system using immunesuppresant drugs to facilitate immune tolerance towards foreign antigens such as foreign proteins, organ transplants, bone marrow and tissue transplantation. Non limiting examples of immunosuppressant drugs include anti-CD4(GK1.5) antibody, Cyclophosphamide, Azathioprine (Imuran), Mycophenolate mofetil (Cellcept), Cyclosporine (Neoral, Sandimmune, Gengraf), Methotrexate (Rheumatrex), Leflunomide (Arava), Cyclophosphamide (Cytoxan) and Chlorambucil (Leukeran).

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Treatment

The present disclosure relates to administration of an ENPP1 or ENPP3 agent to treat PAD, which includes administering sNPP1 and sNPP3 polypeptides and fusion proteins thereof to a subject, and to administration of nucleic acids encoding such polypeptides. Sequences of such polypeptides include the following, without limitation.

```
Sequences
ENPP1 Amino Acid Sequence-Wild Type
SEQ ID NO: 1
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Val Leu Ser Leu
65 70 75 80
Val Leu Ser Val Cys Val Leu Thr Thr Ile Leu Gly Cys Ile Phe Gly
                                85 90 95
Leu Lys Pro Ser Cys Ala Lys Glu Val Lys Ser Cys Lys Gly Arg Cys
                      100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp
                915 920 925
Azurocidin-ENPP1-FC
SEQ ID No: 2
MTRLTVLALLAGLLASSRA**APSCAKEVKSCKGRCFERTFGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLOW
LOLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSFNYEGIARNLSCREPNQHFKPYLKHELP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DFSNCLYQDFRIPLSPVHKCSFYKNNTKVSYGELSPPQLNKNSSGIYSEALLTTNIVPMYQSFQVIWRYF
HDTLLRKYAEERNGVNVVSGPVFDEDYDGRCDSLENLRQKRRVIRNQEILIPTHFFIVLTSCKDTSQTPL
HCENLDTLAFILPHRTDNSESCVHGKHDSSWVEELLMLHRARITDVEHITGLSFYQQRKEPVSDILKLKT
HLPTFSQEDLINDKTHTCPPCPAPELLGGPSVFLFPPKPKDTLMISRTPEVTCVVVDVSHEDPEVKENWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPGK
Single underline-Azurocidin signal sequence, Double underline-
Beginning and end of ENPP1 sequence, Bold residues-Fc sequence,
indicates the cleavage point of the signal sequence.
Azurocidin-ENPP1-Alb
SEQ ID No: 3
MTRLTVLALLAGLLASSRA**APSCAKEVKSCKGRCFERTEGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLOW
LQLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSENYEGIARNLSCREPNQHFKPYLKHELP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DFSNCLYQDFRIPLSPVHKCSFYKNNTKVSYGELSPPQLNKNSSGIYSEALLTTNIVPMYQSFQVIWRYF
HDTLLRKYAEERNGVNVVSGPVEDEDYDGRCDSLENLRQKRRVIRNQEILIPTHFFIVLTSCKDTSQTPL
HCENLDTLAFILPHRTDNSESCVHGKHDSSWVEELLMLHRARITDVEHITGLSFYQQRKEPVSDILKLKT
HLPTFSQEDLINMKWVTFLLLLFVSGSAFSRGVFRREAHKSEIAHRYNDLGEQHFKGLVLIAFSQYLQKC
SYDEHAKLVQEVTDFAKTCVADESAANCDKSLHTLFGDKLCAIPNLRENYGELADCCTKOEPERNECFLQ
HKDDNPSLPPFERPEAEAMCTSFKENPTTFMGHYLHEVARRHPYFYAPELLYYAEQYNEILTQCCAEADK
ESCLTPKLDGVKEKALVSSVRQRMKCSSMQKFGERAFKAWAVARLSQTFPNADFAEITKLATDLTKVNKE
CCHGDLLECADDRAELAKYMCENQATISSKLQTCCDKPLLKKAHCLSEVEHDTMPADLPAIAADFVEDQE
VCKNYAEAKDVFLGTFLYEYSRRHPDYSVSLLLRLAKKYEATLEKCCAEANPPACYGTVLAEFQPLVEEP
KNLVKTNCDLYEKLGEYGFQNAILVRYTQKAPQVSTPTLVEAARNLGRVGTKCCTLPEDORLPCVEDYLS
AILNRVCLLHEKTPVSEHVTKCCSGSLVERRPCFSALTVDETYVPKEFKAETFTFHSDICTLPEKEKQIK
KQTALAELVKHKPKATAEQLKTVMDDFAQFLDTCCKAADKDTCFSTEGPNLVTRCKDALARSWSHPQFEK
Single underline-Azurocidin signal sequence, Double underline-
Beginning and end of ENPP1 sequence, Bold residues-Albumin sequence,
** indicates the cleavage point of the signal sequence.
Azurocidin-ENPP1
SEQ ID No: 4
MTRLTVLALLAGLLASSRA**APSCAKEVKSCKGRCFERTFGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLOW
LQLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSENYEGIARNLSCREPNQHFKPYLKHELP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DESNCLYQDFRIPLSPVHKCSFYKNNTKVSYGELSPPQLNKNSSGIYSEALLTTNIVPMYQSFQVIWRYF
HDTLLRKYAEERNGVNVVSGPVEDEDYDGRCDSLENLRQKRRVIRNQEILIPTHFFIVLTSCKDTSQTAP
SCAKEVKSCKGRCFERTFGNCRCDAACVELGNCCLDYQETCIEPEHIWTCNKFRCGEKRLTRSLCACSDD
CKDKGDCCINYSSVCQGEKSWVEEPCESINEPQCPAGFETPPTLLESLDGFRAEYLHTWGGLLPVISKLK
KCGTYTKNMRPVYPTKTFPNHYSIVTGLYPESHGIIDNKMYDPKMNASFSLKSKEKENPEWYKGEPIWVT
AKYQGLKSGTFFWPGSDVEINGIFPDIYKMYNGSVPFEERILAVLQWLQLPKDERPHFYTLYLEEPDSSG
HSYGPVSSEVIKALQRVDGMVGMLMDGLKELNLHRCLNLILISDHGMEQGSCKKYIYLNKYLGDVKNIKV
IYGPAARLRPSDVPDKYYSENYEGIARNLSCREPNQHFKPYLKHELPKRLHFAKSDRIEPLTFYLDPQWQ
LALNPSERKYCGSGFHGSDNVFSNMQALFVGYGPGFKHGIEADTFENIEVYNLMCDLLNLTPAPNNGTHG
SLNHLLKNPVYTPKHPKEVHPLVQCPFTRNPRDNLGCSCNPSILPIEDFQTQENLTVAEEKIIKHETLPY
GRPRVLQKENTICLLSQHQFMSGYSQDILMPLWTSYTVDRNDSESTEDESNCLYQDFRIPLSPVHKCSFY
KNNTKVSYGFLSPPQLNKNSSGIYSEALLTTNIVPMYQSFQVIWRYFHDTLLRKYAEERNGVNVVSGPVF
DEDYDGRCDSLENLRQKRRVIRNQEILIPTHFFIVLTSCKDTSQTPLHCENLDTLAFILPHRTDNSESCV
HGKHDSSWVEELLMLHRARITDVEHITGLSFYQQRKEPVSDILKLKTHLPTFSQED
Single underline-Azurocidin signal sequence, Double underline-
Beginning and end of ENPP1 sequence,**indicates the cleavage point
of the signal sequence.
ENPP2 Amino Acid Sequence-Wild Type
SEQ ID NO: 5
Met Ala Arg Arg Ser Ser Phe Gln Ser Cys Gln Ile Ile Ser Leu Phe
1 5 10 15
Thr Phe Ala Val Gly Val Asn Ile Cys Leu Gly Phe Thr Ala His Arg
                        20 25 30
Ile Lys Arg Ala Glu Gly Trp Glu Glu Gly Pro Pro Thr Val Leu Ser
                35 40 45
Asp Ser Pro Trp Thr Asn Ile Ser Gly Ser Cys Lys Gly Arg Cys Phe
        50 55 60
Glu Leu Gln Glu Ala Gly Pro Pro Asp Cys Arg Cys Asp Asn Leu Cys
65 70 75 80
Lys Ser Tyr Thr Ser Cys Cys His Asp Phe Asp Glu Leu Cys Leu Lys
                                85 90 95
Thr Ala Arg Gly Trp Glu Cys Thr Lys Asp Arg Cys Gly Glu Val Arg
                      100 105 110
Asn Glu Glu Asn Ala Cys His Cys Ser Glu Asp Cys Leu Ala Arg Gly
                115 120 125
Asp Cys Cys Thr Asn Tyr Gln Val Val Cys Lys Gly Glu Ser His Trp
        130 135 140
Val Asp Asp Asp Cys Glu Glu Ile Lys Ala Ala Glu Cys Pro Ala Gly
145 150 155 160
Phe Val Arg Pro Pro Leu Ile Ile Phe Ser Val Asp Gly Phe Arg Ala
                                165 170 175
Ser Tyr Met Lys Lys Gly Ser Lys Val Met Pro Asn Ile Glu Lys Leu
                        180 185 190
Arg Ser Cys Gly Thr His Ser Pro Tyr Met Arg Pro Val Tyr Pro Thr
                195 200 205
Lys Thr Phe Pro Asn Leu Tyr Thr Leu Ala Thr Gly Leu Tyr Pro Glu
        210 215 220
Ser His Gly Ile Val Gly Asn Ser Met Tyr Asp Pro Val Phe Asp Ala
225 230 235 240
Thr Phe His Leu Arg Gly Arg Glu Lys Phe Asn His Arg Trp Trp Gly
                                245 250 255
Gly Gln Pro Leu Trp Ile Thr Ala Thr Lys Gln Gly Val Lys Ala Gly
                        260 265 270
Thr Phe Phe Trp Ser Val Val Ile Pro His Glu Arg Arg Ile Leu Thr
                275 280 285
Ile Leu Gln Trp Leu Thr Leu Pro Asp His Glu Arg Pro Ser Val Tyr
        290 295 300
Ala Phe Tyr Ser Glu Gln Pro Asp Phe Ser Gly His Lys Tyr Gly Pro
305 310 315 320
Phe Gly Pro Glu Met Thr Asn Pro Leu Arg Glu Ile Asp Lys Ile Val
                                325 330 335
Gly Gln Leu Met Asp Gly Leu Lys Gln Leu Lys Leu His Arg Cys Val
                        340 345 350
Asn Val Ile Phe Val Gly Asp His Gly Met Glu Asp Val Thr Cys Asp
                355 360 365
Arg Thr Glu Phe Leu Ser Asn Tyr Leu Thr Asn Val Asp Asp Ile Thr
        370 375 380
Leu Val Pro Gly Thr Leu Gly Arg Ile Arg Ser Lys Phe Ser Asn Asn
385 390 395 400
Ala Lys Tyr Asp Pro Lys Ala Ile Ile Ala Asn Leu Thr Cys Lys Lys
                                405 410 415
Pro Asp Gln His Phe Lys Pro Tyr Leu Lys Gln His Leu Pro Lys Arg
                        420 425 430
Leu His Tyr Ala Asn Asn Arg Arg Ile Glu Asp Ile His Leu Leu Val
                435 440 445
Glu Arg Arg Trp His Val Ala Arg Lys Pro Leu Asp Val Tyr Lys Lys
        450 455 460
Pro Ser Gly Lys Cys Phe Phe Gln Gly Asp His Gly Phe Asp Asn Lys
465 470 475 480
Val Asn Ser Met Gln Thr Val Phe Val Gly Tyr Gly Ser Thr Phe Lys
                                485 490 495
Tyr Lys Thr Lys Val Pro Pro Phe Glu Asn Ile Glu Leu Tyr Asn Val
                        500 505 510
Met Cys Asp Leu Leu Gly Leu Lys Pro Ala Pro Asn Asn Gly Thr His
                515 520 525
Gly Ser Leu Asn His Leu Leu Arg Thr Asn Thr Phe Arg Pro Thr Met
        530 535 540
Pro Glu Glu Val Thr Arg Pro Asn Tyr Pro Gly Ile Met Tyr Leu Gln
545 550 555 560
Ser Asp Phe Asp Leu Gly Cys Thr Cys Asp Asp Lys Val Glu Pro Lys
                                565 570 575
Asn Lys Leu Asp Glu Leu Asn Lys Arg Leu His Thr Lys Gly Ser Thr
                        580 585 590
Glu Ala Glu Thr Arg Lys Phe Arg Gly Ser Arg Asn Glu Asn Lys Glu
                595 600 605
Asn Ile Asn Gly Asn Phe Glu Pro Arg Lys Glu Arg His Leu Leu Tyr
        610 615 620
Gly Arg Pro Ala Val Leu Tyr Arg Thr Arg Tyr Asp Ile Leu Tyr His
625 630 635 640
Thr Asp Phe Glu Ser Gly Tyr Ser Glu Ile Phe Leu Met Pro Leu Trp
                                645 650 655
Thr Ser Tyr Thr Val Ser Lys Gln Ala Glu Val Ser Ser Val Pro Asp
                        660 665 670
His Leu Thr Ser Cys Val Arg Pro Asp Val Arg Val Ser Pro Ser Phe
                675 680 685
Ser Gln Asn Cys Leu Ala Tyr Lys Asn Asp Lys Gln Met Ser Tyr Gly
        690 695 700
Phe Leu Phe Pro Pro Tyr Leu Ser Ser Ser Pro Glu Ala Lys Tyr Asp
705 710 715 720
Ala Phe Leu Val Thr Asn Met Val Pro Met Tyr Pro Ala Phe Lys Arg
                                725 730 735
Val Trp Asn Tyr Phe Gln Arg Val Leu Val Lys Lys Tyr Ala Ser Glu
                        740 745 750
Arg Asn Gly Val Asn Val Ile Ser Gly Pro Ile Phe Asp Tyr Asp Tyr
                755 760 765
Asp Gly Leu His Asp Thr Glu Asp Lys Ile Lys Gln Tyr Val Glu Gly
        770 775 780
Ser Ser Ile Pro Val Pro Thr His Tyr Tyr Ser Ile Ile Thr Ser Cys
785 790 795 800
Leu Asp Phe Thr Gln Pro Ala Asp Lys Cys Asp Gly Pro Leu Ser Val
                                805 810 815
Ser Ser Phe Ile Leu Pro His Arg Pro Asp Asn Glu Glu Ser Cys Asn
                        820 825 830
Ser Ser Glu Asp Glu Ser Lys Trp Val Glu Glu Leu Met Lys Met His
                835 840 845
Thr Ala Arg Val Arg Asp Ile Glu His Leu Thr Ser Leu Asp Phe Phe
        850 855 860
Arg Lys Thr Ser Arg Ser Tyr Pro Glu Ile Leu Thr Leu Lys Thr Tyr
865 870 875 880
Leu His Thr Tyr Glu Ser Glu Ile
                                885
Extracellular Domain of ENPP 3:
SEQ. ID NO: 6
Glu Lys Gln Gly Ser Cys Arg Lys Lys Cys Phe Asp Ala Ser Phe Arg
1 5 10 15
Gly Leu Glu Asn Cys Arg Cys Asp Val Ala Cys Lys Asp Arg Gly Asp
                        20 25 30
Cys Cys Trp Asp Phe Glu Asp Thr Cys Val Glu Ser Thr Arg Ile Trp
                35 40 45
Met Cys Asn Lys Phe Arg Cys Gly Glu Thr Arg Leu Glu Ala Ser Leu
        50 55 60
Cys Ser Cys Ser Asp Asp Cys Leu Gln Arg Lys Asp Cys Cys Ala Asp
65 70 75 80
Tyr Lys Ser Val Cys Gln Gly Glu Thr Ser Trp Leu Glu Glu Asn Cys
                                85 90 95
Asp Thr Ala Gln Gln Ser Gln Cys Pro Glu Gly Phe Asp Leu Pro Pro
                      100 105 110
Val Ile Leu Phe Ser Met Asp Gly Phe Arg Ala Glu Tyr Leu Tyr Thr
                115 120 125
Trp Asp Thr Leu Met Pro Asn Ile Asn Lys Leu Lys Thr Cys Gly Ile
        130 135 140
His Ser Lys Tyr Met Arg Ala Met Tyr Pro Thr Lys Thr Phe Pro Asn
145 150 155 160
His Tyr Thr Ile Val Thr Gly Leu Tyr Pro Glu Ser His Gly Ile Ile
                                165 170 175
Asp Asn Asn Met Tyr Asp Val Asn Leu Asn Lys Asn Phe Ser Leu Ser
                        180 185 190
Ser Lys Glu Gln Asn Asn Pro Ala Trp Trp His Gly Gln Pro Met Trp
                195 200 205
Leu Thr Ala Met Tyr Gln Gly Leu Lys Ala Ala Thr Tyr Phe Trp Pro
        210 215 220
Gly Ser Glu Val Ala Ile Asn Gly Ser Phe Pro Ser Ile Tyr Met Pro
225 230 235 240
Tyr Asn Gly Ser Val Pro Phe Glu Glu Arg Ile Ser Thr Leu Leu Lys
                                245 250 255
Trp Leu Asp Leu Pro Lys Ala Glu Arg Pro Arg Phe Tyr Thr Met Tyr
                        260 265 270
Phe Glu Glu Pro Asp Ser Ser Gly His Ala Gly Gly Pro Val Ser Ala
                275 280 285
Arg Val Ile Lys Ala Leu Gln Val Val Asp His Ala Phe Gly Met Leu
        290 295 300
Met Glu Gly Leu Lys Gln Arg Asn Leu His Asn Cys Val Asn Ile Ile
305 310 315 320
Leu Leu Ala Asp His Gly Met Asp Gln Thr Tyr Cys Asn Lys Met Glu
                                325 330 335
Tyr Met Thr Asp Tyr Phe Pro Arg Ile Asn Phe Phe Tyr Met Tyr Glu
                        340 345 350
Gly Pro Ala Pro Arg Ile Arg Ala His Asn Ile Pro His Asp Phe Phe
                355 360 365
Ser Phe Asn Ser Glu Glu Ile Val Arg Asn Leu Ser Cys Arg Lys Pro
        370 375 380
Asp Gln His Phe Lys Pro Tyr Leu Thr Pro Asp Leu Pro Lys Arg Leu
385 390 395 400
His Tyr Ala Lys Asn Val Arg Ile Asp Lys Val His Leu Phe Val Asp
                                405 410 415
Gln Gln Trp Leu Ala Val Arg Ser Lys Ser Asn Thr Asn Cys Gly Gly
                        420 425 430
Gly Asn His Gly Tyr Asn Asn Glu Phe Arg Ser Met Glu Ala Ile Phe
                435 440 445
Leu Ala His Gly Pro Ser Phe Lys Glu Lys Thr Glu Val Glu Pro Phe
        450 455 460
Glu Asn Ile Glu Val Tyr Asn Leu Met Cys Asp Leu Leu Arg Ile Gln
465 470 475 480
Pro Ala Pro Asn Asn Gly Thr His Gly Ser Leu Asn His Leu Leu Lys
                                485 490 495
Val Pro Phe Tyr Glu Pro Ser His Ala Glu Glu Val Ser Lys Phe Ser
                        500 505 510
Val Cys Gly Phe Ala Asn Pro Leu Pro Thr Glu Ser Leu Asp Cys Phe
                515 520 525
Cys Pro His Leu Gln Asn Ser Thr Gln Leu Glu Gln Val Asn Gln Met
        530 535 540
Leu Asn Leu Thr Gln Glu Glu Ile Thr Ala Thr Val Lys Val Asn Leu
545 550 555 560
Pro Phe Gly Arg Pro Arg Val Leu Gln Lys Asn Val Asp His Cys Leu
                                565 570 575
Leu Tyr His Arg Glu Tyr Val Ser Gly Phe Gly Lys Ala Met Arg Met
                        580 585 590
Pro Met Trp Ser Ser Tyr Thr Val Pro Gln Leu Gly Asp Thr Ser Pro
                595 600 605
Leu Pro Pro Thr Val Pro Asp Cys Leu Arg Ala Asp Val Arg Val Pro
        610 615 620
Pro Ser Glu Ser Gln Lys Cys Ser Phe Tyr Leu Ala Asp Lys Asn Ile
625 630 635 640
Thr His Gly Phe Leu Tyr Pro Pro Ala Ser Asn Arg Thr Ser Asp Ser
                                645 650 655
Gln Tyr Asp Ala Leu Ile Thr Ser Asn Leu Val Pro Met Tyr Glu Glu
                        660 665 670
Phe Arg Lys Met Trp Asp Tyr Phe His Ser Val Leu Leu Ile Lys His
                675 680 685
Ala Thr Glu Arg Asn Gly Val Asn Val Val Ser Gly Pro Ile Phe Asp
        690 695 700
Tyr Asn Tyr Asp Gly His Phe Asp Ala Pro Asp Glu Ile Thr Lys His
705 710 715 720
Leu Ala Asn Thr Asp Val Pro Ile Pro Thr His Tyr Phe Val Val Leu
                                725 730 735
Thr Ser Cys Lys Asn Lys Ser His Thr Pro Glu Asn Cys Pro Gly Trp
                        740 745 750
Leu Asp Val Leu Pro Phe Ile Ile Pro His Arg Pro Thr Asn Val Glu
                755 760 765
Ser Cys Pro Glu Gly Lys Pro Glu Ala Leu Trp Val Glu Glu Arg Phe
        770 775 780
Thr Ala His Ile Ala Arg Val Arg Asp Val Glu Leu Leu Thr Gly Leu
785 790 795 800
Asp Phe Tyr Gln Asp Lys Val Gln Pro Val Ser Glu Ile Leu Gln Leu
                                805 810 815
Lys Thr Tyr Leu Pro Thr Phe Glu Thr Thr Ile
                        820 825
NPP3 Amino Acid Sequence:
SEQ. ID NO: 7
Met Glu Ser Thr Leu Thr Leu Ala Thr Glu Gln Pro Val Lys Lys Asn
1 5 10 15
Thr Leu Lys Lys Tyr Lys Ile Ala Cys Ile Val Leu Leu Ala Leu Leu
                        20 25 30
Val Ile Met Ser Leu Gly Leu Gly Leu Gly Leu Gly Leu Arg Lys Leu
                35 40 45
Glu Lys Gln Gly Ser Cys Arg Lys Lys Cys Phe Asp Ala Ser Phe Arg
        50 55 60
Gly Leu Glu Asn Cys Arg Cys Asp Val Ala Cys Lys Asp Arg Gly Asp
65 70 75 80
Cys Cys Trp Asp Phe Glu Asp Thr Cys Val Glu Ser Thr Arg Ile Trp
                                85 90 95
Met Cys Asn Lys Phe Arg Cys Gly Glu Thr Arg Leu Glu Ala Ser Leu
                      100 105 110
Cys Ser Cys Ser Asp Asp Cys Leu Gln Arg Lys Asp Cys Cys Ala Asp
                115 120 125
Tyr Lys Ser Val Cys Gln Gly Glu Thr Ser Trp Leu Glu Glu Asn Cys
        130 135 140
Asp Thr Ala Gln Gln Ser Gln Cys Pro Glu Gly Phe Asp Leu Pro Pro
145 150 155 160
Val Ile Leu Phe Ser Met Asp Gly Phe Arg Ala Glu Tyr Leu Tyr Thr
                                165 170 175
Trp Asp Thr Leu Met Pro Asn Ile Asn Lys Leu Lys Thr Cys Gly Ile
                        180 185 190
His Ser Lys Tyr Met Arg Ala Met Tyr Pro Thr Lys Thr Phe Pro Asn
                195 200 205
His Tyr Thr Ile Val Thr Gly Leu Tyr Pro Glu Ser His Gly Ile Ile
        210 215 220
Asp Asn Asn Met Tyr Asp Val Asn Leu Asn Lys Asn Phe Ser Leu Ser
225 230 235 240
Ser Lys Glu Gln Asn Asn Pro Ala Trp Trp His Gly Gln Pro Met Trp
                                245 250 255
Leu Thr Ala Met Tyr Gln Gly Leu Lys Ala Ala Thr Tyr Phe Trp Pro
                        260 265 270
Gly Ser Glu Val Ala Ile Asn Gly Ser Phe Pro Ser Ile Tyr Met Pro
                275 280 285
Tyr Asn Gly Ser Val Pro Phe Glu Glu Arg Ile Ser Thr Leu Leu Lys
        290 295 300
Trp Leu Asp Leu Pro Lys Ala Glu Arg Pro Arg Phe Tyr Thr Met Tyr
305 310 315 320
Phe Glu Glu Pro Asp Ser Ser Gly His Ala Gly Gly Pro Val Ser Ala
                                325 330 335
Arg Val Ile Lys Ala Leu Gln Val Val Asp His Ala Phe Gly Met Leu
                        340 345 350
Met Glu Gly Leu Lys Gln Arg Asn Leu His Asn Cys Val Asn Ile Ile
                355 360 365
Leu Leu Ala Asp His Gly Met Asp Gln Thr Tyr Cys Asn Lys Met Glu
        370 375 380
Tyr Met Thr Asp Tyr Phe Pro Arg Ile Asn Phe Phe Tyr Met Tyr Glu
385 390 395 400
Gly Pro Ala Pro Arg Ile Arg Ala His Asn Ile Pro His Asp Phe Phe
                                405 410 415
Ser Phe Asn Ser Glu Glu Ile Val Arg Asn Leu Ser Cys Arg Lys Pro
                        420 425 430
Asp Gln His Phe Lys Pro Tyr Leu Thr Pro Asp Leu Pro Lys Arg Leu
                435 440 445
His Tyr Ala Lys Asn Val Arg Ile Asp Lys Val His Leu Phe Val Asp
        450 455 460
Gln Gln Trp Leu Ala Val Arg Ser Lys Ser Asn Thr Asn Cys Gly Gly
465 470 475 480
Gly Asn His Gly Tyr Asn Asn Glu Phe Arg Ser Met Glu Ala Ile Phe
                                485 490 495
Leu Ala His Gly Pro Ser Phe Lys Glu Lys Thr Glu Val Glu Pro Phe
                        500 505 510
Glu Asn Ile Glu Val Tyr Asn Leu Met Cys Asp Leu Leu Arg Ile Gln
                515 520 525
Glu Asn Ile Glu Val Tyr Asn Leu Met Cys Asp Leu Leu Arg Ile Gln
                515 520 525
Pro Ala Pro Asn Asn Gly Thr His Gly Ser Leu Asn His Leu Leu Lys
        530 535 540
Val Pro Phe Tyr Glu Pro Ser His Ala Glu Glu Val Ser Lys Phe Ser
545 550 555 560
Val Cys Gly Phe Ala Asn Pro Leu Pro Thr Glu Ser Leu Asp Cys Phe
                                565 570 575
Cys Pro His Leu Gln Asn Ser Thr Gln Leu Glu Gln Val Asn Gln Met
                        580 585 590
Leu Asn Leu Thr Gln Glu Glu Ile Thr Ala Thr Val Lys Val Asn Leu
                595 600 605
Pro Phe Gly Arg Pro Arg Val Leu Gln Lys Asn Val Asp His Cys Leu
        610 615 620
Leu Tyr His Arg Glu Tyr Val Ser Gly Phe Gly Lys Ala Met Arg Met
625 630 635 640
Pro Met Trp Ser Ser Tyr Thr Val Pro Gln Leu Gly Asp Thr Ser Pro
                                645 650 655
Leu Pro Pro Thr Val Pro Asp Cys Leu Arg Ala Asp Val Arg Val Pro
                        660 665 670
Pro Ser Glu Ser Gln Lys Cys Ser Phe Tyr Leu Ala Asp Lys Asn Ile
                675 680 685
Thr His Gly Phe Leu Tyr Pro Pro Ala Ser Asn Arg Thr Ser Asp Ser
        690 695 700
Gln Tyr Asp Ala Leu Ile Thr Ser Asn Leu Val Pro Met Tyr Glu Glu
705 710 715 720
Phe Arg Lys Met Trp Asp Tyr Phe His Ser Val Leu Leu Ile Lys His
                                725 730 735
Ala Thr Glu Arg Asn Gly Val Asn Val Val Ser Gly Pro Ile Phe Asp
                        740 745 750
Tyr Asn Tyr Asp Gly His Phe Asp Ala Pro Asp Glu Ile Thr Lys His
                755 760 765
Leu Ala Asn Thr Asp Val Pro Ile Pro Thr His Tyr Phe Val Val Leu
        770 775 780
Thr Ser Cys Lys Asn Lys Ser His Thr Pro Glu Asn Cys Pro Gly Trp
785 790 795 800
Leu Asp Val Leu Pro Phe Ile Ile Pro His Arg Pro Thr Asn Val Glu
                                805 810 815
Ser Cys Pro Glu Gly Lys Pro Glu Ala Leu Trp Val Glu Glu Arg Phe
                        820 825 830
Thr Ala His Ile Ala Arg Val Arg Asp Val Glu Leu Leu Thr Gly Leu
                835 840 845
Asp Phe Tyr Gln Asp Lys Val Gln Pro Val Ser Glu Ile Leu Gln Leu
        850 855 860
Lys Thr Tyr Leu Pro Thr Phe Glu Thr Thr Ile
865 870 875
Azurocidin-ENPP3-FC
SEQ ID No: 8
MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGFDLPPVI
LESMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSFPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEGPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKESVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEFR
KMWDYFHSVLLIKHATERNGVNVVSGPIFDYNYDGHFDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTIDKTHTCPPCPAPELLGGPSVFLFPPKPKDTLMISRTPEVTCVVVDVSHEDPEVKENW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
Single underline-Azurocidin signal sequence, Double underline-
Beginning and end of ENPP3 sequence, Bold residues-Fc sequence,
** indicates the cleavage point of the signal sequence.
Azurocidin-ENPP3-Albumin
SEQ ID No: 9
MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGEDLPPVI
LESMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSFPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEGPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKFSVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEER
KMWDYFHSVLLIKHATERNGVNVVSGPIFDYNYDGHEDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTIMKWVTFLLLLFVSGSAFSRGVFRREAHKSEIAHRYNDLGEQHFKGLVLIAFSQYLQK
CSYDEHAKLVQEVTDFAKTCVADESAANCDKSLHTLFGDKLCAIPNLRENYGELADCCTKQEPERNECFL
QHKDDNPSLPPFERPEAEAMCTSFKENPTTEMGHYLHEVARRHPYFYAPELLYYAEQYNEILTQCCAEAD
KESCLTPKLDGVKEKALVSSVRQRMKCSSMQKFGERAFKAWAVARLSQTFPNADFAEITKLATDLTKVNK
ECCHGDLLECADDRAELAKYMCENQATISSKLQTCCDKPLLKKAHCLSEVEHDTMPADLPAIAADFVEDQ
EVCKNYAEAKDVFLGTFLYEYSRRHPDYSVSLLLRLAKKYEATLEKCCAEANPPACYGTVLAEFQPLVEE
PKNLVKTNCDLYEKLGEYGFQNAILVRYTQKAPQVSTPTLVEAARNLGRVGTKCCTLPEDQRLPCVEDYL
SAILNRVCLLHEKTPVSEHVTKCCSGSLVERRPCFSALTVDETYVPKEFKAETFTFHSDICTLPEKEKQI
KKQTALAELVKHKPKATAEQLKTVMDDFAQFLDTCCKAADKDTCFSTEGPNLVTRCKDALARSWSHPQFE
K
Single underline-Azurocidin signal sequence, Double underline-
Beginning and end of ENPP3 sequence, Bold residues-Albumin sequence,
** indicates the cleavage point of the signal sequence.
Azurocidin-ENPP3
SEQ ID No: 10
MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGEDLPPVI
LFSMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSFPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKESVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEFR
KMWDYFHSVLLIKHATERNGVNVVSGPIFDYNYDGHFDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTI
Single underline-Azurocidin signal sequence, Double underline-
Beginning and end of ENPP3 sequence,**indicates the cleavage point
of the signal sequence.
ENPP4 Amino Acid Sequence-Wild Type
SEQ. ID NO: 11
Met Lys Leu Leu Val Ile Leu Leu Phe Ser Gly Leu Ile Thr Gly Phe
1 5 10 15
Arg Ser Asp Ser Ser Ser Ser Leu Pro Pro Lys Leu Leu Leu Val Ser
                        20 25 30
Phe Asp Gly Phe Arg Ala Asp Tyr Leu Lys Asn Tyr Glu Phe Pro His
                35 40 45
Leu Gln Asn Phe Ile Lys Glu Gly Val Leu Val Glu His Val Lys Asn
        50 55 60
Val Phe Ile Thr Lys Thr Phe Pro Asn His Tyr Ser Ile Val Thr Gly
65 70 75 80
Leu Tyr Glu Glu Ser His Gly Ile Val Ala Asn Ser Met Tyr Asp Ala
                                85 90 95
Val Thr Lys Lys His Phe Ser Asp Ser Asn Asp Lys Asp Pro Phe Trp
                      100 105 110
Trp Asn Glu Ala Val Pro Ile Trp Val Thr Asn Gln Leu Gln Glu Asn
                115 120 125
Arg Ser Ser Ala Ala Ala Met Trp Pro Gly Thr Asp Val Pro Ile His
        130 135 140
Asp Thr Ile Ser Ser Tyr Phe Met Asn Tyr Asn Ser Ser Val Ser Phe
145 150 155 160
Glu Glu Arg Leu Asn Asn Ile Thr Met Trp Leu Asn Asn Ser Asn Pro
                                165 170 175
Pro Val Thr Phe Ala Thr Leu Tyr Trp Glu Glu Pro Asp Ala Ser Gly
                        180 185 190
His Lys Tyr Gly Pro Glu Asp Lys Glu Asn Met Ser Arg Val Leu Lys
                195 200 205
Lys Ile Asp Asp Leu Ile Gly Asp Leu Val Gln Arg Leu Lys Met Leu
        210 215 220
Gly Leu Trp Glu Asn Leu Asn Val Ile Ile Thr Ser Asp His Gly Met
225 230 235 240
Thr Gln Cys Ser Gln Asp Arg Leu Ile Asn Leu Asp Ser Cys Ile Asp
                                245 250 255
His Ser Tyr Tyr Thr Leu Ile Asp Leu Ser Pro Val Ala Ala Ile Leu
                        260 265 270
Pro Lys Ile Asn Arg Thr Glu Val Tyr Asn Lys Leu Lys Asn Cys Ser
                275 280 285
Pro His Met Asn Val Tyr Leu Lys Glu Asp Ile Pro Asn Arg Phe Tyr
        290 295 300
Tyr Gln His Asn Asp Arg Ile Gln Pro Ile Ile Leu Val Ala Asp Glu
305 310 315 320
Gly Trp Thr Ile Val Leu Asn Glu Ser Ser Gln Lys Leu Gly Asp His
                                325 330 335
Gly Tyr Asp Asn Ser Leu Pro Ser Met His Pro Phe Leu Ala Ala His
                        340 345 350
Gly Pro Ala Phe His Lys Gly Tyr Lys His Ser Thr Ile Asn Ile Val
                355 360 365
Asp Ile Tyr Pro Met Met Cys His Ile Leu Gly Leu Lys Pro His Pro
        370 375 380
Asn Asn Gly Thr Phe Gly His Thr Lys Cys Leu Leu Val Asp Gln Trp
385 390 395 400
Cys Ile Asn Leu Pro Glu Ala Ile Ala Ile Val Ile Gly Ser Leu Leu
                                405 410 415
Val Leu Thr Met Leu Thr Cys Leu Ile Ile Ile Met Gln Asn Arg Leu
                        420 425 430
Ser Val Pro Arg Pro Phe Ser Arg Leu Gln Leu Gln Glu Asp Asp Asp
                435 440 445
Asp Pro Leu Ile Gly
        450
ENPP51 Amino Acid Sequence
SEQ. ID NO: 12
Met Thr Ser Lys Phe Leu Leu Val Ser Phe Ile Leu Ala Ala Leu Ser
1 5 10 15
Leu Ser Thr Thr Phe Ser Leu Gln**Pro Ser Cys Ala Lys Glu Val Lys
                        20 25 30
Ser Cys Lys Gly Arg Cys Phe Glu Arg Thr Phe Ser Asn Cys Arg Cys
                35 40 45
Asp Ala Ala Cys Val Ser Leu Gly Asn Cys Cys Leu Asp Phe Gln Glu
        50 55 60
Thr Cys Val Glu Pro Thr His Ile Trp Thr Cys Asn Lys Phe Arg Cys
65 70 75 80
Gly Glu Lys Arg Leu Ser Arg Phe Val Cys Ser Cys Ala Asp Asp Cys
                                85 90 95
Lys Thr His Asn Asp Cys Cys Ile Asn Tyr Ser Ser Val Cys Gln Asp
                      100 105 110
Lys Lys Ser Trp Val Glu Glu Thr Cys Glu Ser Ile Asp Thr Pro Glu
                115 120 125
Cys Pro Ala Glu Phe Glu Ser Pro Pro Thr Leu Leu Phe Ser Leu Asp
        130 135 140
Gly Phe Arg Ala Glu Tyr Leu His Thr Trp Gly Gly Leu Leu Pro Val
145 150 155 160
Ile Ser Lys Leu Lys Asn Cys Gly Thr Tyr Thr Lys Asn Met Arg Pro
                                165 170 175
Met Tyr Pro Thr Lys Thr Phe Pro Asn His Tyr Ser Ile Val Thr Gly
                        180 185 190
Leu Tyr Pro Glu Ser His Gly Ile Ile Asp Asn Lys Met Tyr Asp Pro
                195 200 205
Lys Met Asn Ala Ser Phe Ser Leu Lys Ser Lys Glu Lys Phe Asn Pro
        210 215 220
Leu Trp Tyr Lys Gly Gln Pro Ile Trp Val Thr Ala Asn His Gln Glu
225 230 235 240
Val Lys Ser Gly Thr Tyr Phe Trp Pro Gly Ser Asp Val Glu Ile Asp
                                245 250 255
Gly Ile Leu Pro Asp Ile Tyr Lys Val Tyr Asn Gly Ser Val Pro Phe
                        260 265 270
Glu Glu Arg Ile Leu Ala Val Leu Glu Trp Leu Gln Leu Pro Ser His
                275 280 285
Glu Arg Pro His Phe Tyr Thr Leu Tyr Leu Glu Glu Pro Asp Ser Ser
        290 295 300
Gly His Ser His Gly Pro Val Ser Ser Glu Val Ile Lys Ala Leu Gln
305 310 315 320
Lys Val Asp Arg Leu Val Gly Met Leu Met Asp Gly Leu Lys Asp Leu
                                325 330 335
Gly Leu Asp Lys Cys Leu Asn Leu Ile Leu Ile Ser Asp His Gly Met
                        340 345 350
Glu Gln Gly Ser Cys Lys Lys Tyr Val Tyr Leu Asn Lys Tyr Leu Gly
                355 360 365
Asp Val Asn Asn Val Lys Val Val Tyr Gly Pro Ala Ala Arg Leu Arg
        370 375 380
Pro Thr Asp Val Pro Glu Thr Tyr Tyr Ser Phe Asn Tyr Glu Ala Leu
385 390 395 400
Ala Lys Asn Leu Ser Cys Arg Glu Pro Asn Gln His Phe Arg Pro Tyr
                                405 410 415
Leu Lys Pro Phe Leu Pro Lys Arg Leu His Phe Ala Lys Ser Asp Arg
                        420 425 430
Ile Glu Pro Leu Thr Phe Tyr Leu Asp Pro Gln Trp Gln Leu Ala Leu
                435 440 445
Asn Pro Ser Glu Arg Lys Tyr Cys Gly Ser Gly Phe His Gly Ser Asp
        450 455 460
Asn Leu Phe Ser Asn Met Gln Ala Leu Phe Ile Gly Tyr Gly Pro Ala
465 470 475 480
Phe Lys His Gly Ala Glu Val Asp Ser Phe Glu Asn Ile Glu Val Tyr
                                485 490 495
Asn Leu Met Cys Asp Leu Leu Gly Leu Ile Pro Ala Pro Asn Asn Gly
                        500 505 510
Ser His Gly Ser Leu Asn His Leu Leu Lys Lys Pro Ile Tyr Asn Pro
                515 520 525
Ser His Pro Lys Glu Glu Gly Phe Leu Ser Gln Cys Pro Ile Lys Ser
        530 535 540
Thr Ser Asn Asp Leu Gly Cys Thr Cys Asp Pro Trp Ile Val Pro Ile
545 550 555 560
Lys Asp Phe Glu Lys Gln Leu Asn Leu Thr Thr Glu Asp Val Asp Asp
                                565 570 575
Ile Tyr His Met Thr Val Pro Tyr Gly Arg Pro Arg Ile Leu Leu Lys
                        580 585 590
Gln His Arg Val Cys Leu Leu Gln Gln Gln Gln Phe Leu Thr Gly Tyr
                595 600 605
Ser Leu Asp Leu Leu Met Pro Leu Trp Ala Ser Tyr Thr Phe Leu Ser
        610 615 620
Asn Asp Gln Phe Ser Arg Asp Asp Phe Ser Asn Cys Leu Tyr Gln Asp
625 630 635 640
Leu Arg Ile Pro Leu Ser Pro Val His Lys Cys Ser Tyr Tyr Lys Ser
                                645 650 655
Asn Ser Lys Leu Ser Tyr Gly Phe Leu Thr Pro Pro Arg Leu Asn Arg
                        660 665 670
Val Ser Asn His Ile Tyr Ser Glu Ala Leu Leu Thr Ser Asn Ile Val
                675 680 685
Pro Met Tyr Gln Ser Phe Gln Val Ile Trp His Tyr Leu His Asp Thr
        690 695 700
Leu Leu Gln Arg Tyr Ala His Glu Arg Asn Gly Ile Asn Val Val Ser
705 710 715 720
Gly Pro Val Phe Asp Phe Asp Tyr Asp Gly Arg Tyr Asp Ser Leu Glu
                                725 730 735
Ile Leu Lys Gln Asn Ser Arg Val Ile Arg Ser Gln Glu Ile Leu Ile
                        740 745 750
Pro Thr His Phe Phe Ile Val Leu Thr Ser Cys Lys Gln Leu Ser Glu
                755 760 765
Thr Pro Leu Glu Cys Ser Ala Leu Glu Ser Ser Ala Tyr Ile Leu Pro
        770 775 780
His Arg Pro Asp Asn Ile Glu Ser Cys Thr His Gly Lys Arg Glu Ser
785 790 795 800
Ser Trp Val Glu Glu Leu Leu Thr Leu His Arg Ala Arg Val Thr Asp
                                805 810 815
Val Glu Leu Ile Thr Gly Leu Ser Phe Tyr Gln Asp Arg Gln Glu Ser
                        820 825 830
Val Ser Glu Leu Leu Arg Leu Lys Thr His Leu Pro Ile Phe Ser Gln
                835 840 845
Glu Asp
        850
Singly underlined: signal peptide sequence; double-underlined:
beginning and end of NPP1; ** = cleavage position at the signal
peptide sequence
ENPP51
ALB Amino Acid Sequence:
SEQ. ID NO: 13
Met Thr Ser Lys Phe Leu Leu Val Ser Phe Ile Leu Ala Ala Leu Ser
1 5 10 15
Leu Ser Thr Thr Phe Ser Leu Gln**Pro Ser Cys Ala Lys Glu Val Lys
                        20 25 30
Ser Cys Lys Gly Arg Cys Phe Glu Arg Thr Phe Ser Asn Cys Arg Cys
                35 40 45
Asp Ala Ala Cys Val Ser Leu Gly Asn Cys Cys Leu Asp Phe Gln Glu
        50 55 60
Thr Cys Val Glu Pro Thr His Ile Trp Thr Cys Asn Lys Phe Arg Cys
65 70 75 80
Gly Glu Lys Arg Leu Ser Arg Phe Val Cys Ser Cys Ala Asp Asp Cys
                                85 90 95
Lys Thr His Asn Asp Cys Cys Ile Asn Tyr Ser Ser Val Cys Gln Asp
                      100 105 110
Lys Lys Ser Trp Val Glu Glu Thr Cys Glu Ser Ile Asp Thr Pro Glu
                115 120 125
Cys Pro Ala Glu Phe Glu Ser Pro Pro Thr Leu Leu Phe Ser Leu Asp
        130 135 140
Gly Phe Arg Ala Glu Tyr Leu His Thr Trp Gly Gly Leu Leu Pro Val
145 150 155 160
Ile Ser Lys Leu Lys Asn Cys Gly Thr Tyr Thr Lys Asn Met Arg Pro
                                165 170 175
Met Tyr Pro Thr Lys Thr Phe Pro Asn His Tyr Ser Ile Val Thr Gly
                        180 185 190
Leu Tyr Pro Glu Ser His Gly Ile Ile Asp Asn Lys Met Tyr Asp Pro
                195 200 205
Lys Met Asn Ala Ser Phe Ser Leu Lys Ser Lys Glu Lys Phe Asn Pro
        210 215 220
Leu Trp Tyr Lys Gly Gln Pro Ile Trp Val Thr Ala Asn His Gln Glu
225 230 235 240
Val Lys Ser Gly Thr Tyr Phe Trp Pro Gly Ser Asp Val Glu Ile Asp
                                245 250 255
Gly Ile Leu Pro Asp Ile Tyr Lys Val Tyr Asn Gly Ser Val Pro Phe
                        260 265 270
Glu Glu Arg Ile Leu Ala Val Leu Glu Trp Leu Gln Leu Pro Ser His
                275 280 285
Glu Arg Pro His Phe Tyr Thr Leu Tyr Leu Glu Glu Pro Asp Ser Ser
        290 295 300
Gly His Ser His Gly Pro Val Ser Ser Glu Val Ile Lys Ala Leu Gln
305 310 315 320
Lys Val Asp Arg Leu Val Gly Met Leu Met Asp Gly Leu Lys Asp Leu
                                325 330 335
Gly Leu Asp Lys Cys Leu Asn Leu Ile Leu Ile Ser Asp His Gly Met
                        340 345 350
Glu Gln Gly Ser Cys Lys Lys Tyr Val Tyr Leu Asn Lys Tyr Leu Gly
                355 360 365
Asp Val Asn Asn Val Lys Val Val Tyr Gly Pro Ala Ala Arg Leu Arg
        370 375 380
Pro Thr Asp Val Pro Glu Thr Tyr Tyr Ser Phe Asn Tyr Glu Ala Leu
385 390 395 400
Ala Lys Asn Leu Ser Cys Arg Glu Pro Asn Gln His Phe Arg Pro Tyr
                                405 410 415
Leu Lys Pro Phe Leu Pro Lys Arg Leu His Phe Ala Lys Ser Asp Arg
                        420 425 430
Ile Glu Pro Leu Thr Phe Tyr Leu Asp Pro Gln Trp Gln Leu Ala Leu
                435 440 445
Asn Pro Ser Glu Arg Lys Tyr Cys Gly Ser Gly Phe His Gly Ser Asp
        450 455 460
Asn Leu Phe Ser Asn Met Gln Ala Leu Phe Ile Gly Tyr Gly Pro Ala
465 470 475 480
Phe Lys His Gly Ala Glu Val Asp Ser Phe Glu Asn Ile Glu Val Tyr
                                485 490 495
Asn Leu Met Cys Asp Leu Leu Gly Leu Ile Pro Ala Pro Asn Asn Gly
                        500 505 510
Ser His Gly Ser Leu Asn His Leu Leu Lys Lys Pro Ile Tyr Asn Pro
                515 520 525
Ser His Pro Lys Glu Glu Gly Phe Leu Ser Gln Cys Pro Ile Lys Ser
        530 535 540
Thr Ser Asn Asp Leu Gly Cys Thr Cys Asp Pro Trp Ile Val Pro Ile
545 550 555 560
Lys Asp Phe Glu Lys Gln Leu Asn Leu Thr Thr Glu Asp Val Asp Asp
                                565 570 575
Ile Tyr His Met Thr Val Pro Tyr Gly Arg Pro Arg Ile Leu Leu Lys
                        580 585 590
Gln His Arg Val Cys Leu Leu Gln Gln Gln Gln Phe Leu Thr Gly Tyr
                595 600 605
Ser Leu Asp Leu Leu Met Pro Leu Trp Ala Ser Tyr Thr Phe Leu Ser
        610 615 620
Asn Asp Gln Phe Ser Arg Asp Asp Phe Ser Asn Cys Leu Tyr Gln Asp
625 630 635 640
Leu Arg Ile Pro Leu Ser Pro Val His Lys Cys Ser Tyr Tyr Lys Ser
                                645 650 655
Asn Ser Lys Leu Ser Tyr Gly Phe Leu Thr Pro Pro Arg Leu Asn Arg
                        660 665 670
Val Ser Asn His Ile Tyr Ser Glu Ala Leu Leu Thr Ser Asn Ile Val
                675 680 685
Pro Met Tyr Gln Ser Phe Gln Val Ile Trp His Tyr Leu His Asp Thr
        690 695 700
Leu Leu Gln Arg Tyr Ala His Glu Arg Asn Gly Ile Asn Val Val Ser
705 710 715 720
Gly Pro Val Phe Asp Phe Asp Tyr Asp Gly Arg Tyr Asp Ser Leu Glu
                                725 730 735
Ile Leu Lys Gln Asn Ser Arg Val Ile Arg Ser Gln Glu Ile Leu Ile
                        740 745 750
Pro Thr His Phe Phe Ile Val Leu Thr Ser Cys Lys Gln Leu Ser Glu
                755 760 765
Thr Pro Leu Glu Cys Ser Ala Leu Glu Ser Ser Ala Tyr Ile Leu Pro
        770 775 780
His Arg Pro Asp Asn Ile Glu Ser Cys Thr His Gly Lys Arg Glu Ser
785 790 795 800
Ser Trp Val Glu Glu Leu Leu Thr Leu His Arg Ala Arg Val Thr Asp
                                805 810 815
Val Glu Leu Ile Thr Gly Leu Ser Phe Tyr Gln Asp Arg Gln Glu Ser
                        820 825 830
Val Ser Glu Leu Leu Arg Leu Lys Thr His Leu Pro Ile Phe Ser Gln
                835 840 845
Glu Asp Gly Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1240 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355 1360 1365
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu Pro Glu
        1385 1390 1395
Lys Glu Lys Gln Ile Lys Lys Gln Thr Ala Leu Ala Glu Leu Val
        1400 1405 1410
Lys His Lys Pro Lys Ala Thr Ala Glu Gln Leu Lys Thr Val Met
        1415 1420 1425
Asp Asp Phe Ala Gln Phe Leu Asp Thr Cys Cys Lys Ala Ala Asp
        1430 1435 1440
Lys Asp Thr Cys Phe Ser Thr Glu Gly Pro Asn Leu Val Thr Arg
        1445 1450 1455
Cys Lys Asp Ala Leu Ala Arg Ser Trp Ser His Pro Gln Phe Glu
        1460 1465 1470
Lys
Singly underlined: signal peptide sequence; double-underlined:
beginning and end of NPP1; ** = cleavage position at the signal
peptide sequence; bold residues indicate albumin sequence
ENPP5-NPP3-Fc sequence
SEQ. ID NO: 14
Met Thr Ser Lys Phe Leu Leu Val Ser Phe Ile Leu Ala Ala Leu Ser
1 5 10 15
Leu Ser Thr Thr Phe Ser**Lys Gln Gly Ser Cys Arg Lys Lys Cys Phe
                        20 25 30
Asp Ala Ser Phe Arg Gly Leu Glu Asn Cys Arg Cys Asp Val Ala Cys
                35 40 45
Lys Asp Arg Gly Asp Cys Cys Trp Asp Phe Glu Asp Thr Cys Val Glu
        50 55 60
Ser Thr Arg Ile Trp Met Cys Asn Lys Phe Arg Cys Gly Glu Arg Leu
65 70 75 80
Glu Ala Ser Leu Cys Ser Cys Ser Asp Asp Cys Leu Gln Arg Lys Asp
                                85 90 95
Cys Cys Ala Asp Tyr Lys Ser Val Cys Gln Gly Glu Thr Ser Trp Leu
                      100 105 110
Glu Glu Asn Cys Asp Thr Ala Gln Gln Ser Gln Cys Pro Glu Gly Phe
                115 120 125
Asp Leu Pro Val Ile Leu Ser Met Gly Arg Pro Phe Asp Phe Ala Glu
        130 135 140
Tyr Leu Tyr Thr Trp Asp Thr Leu Met Pro Asn Ile Asn Lys Leu Lys
145 150 155 160
Thr Cys Gly Ile His Ser Lys Tyr Met Arg Ala Met Tyr Pro Thr Lys
                                165 170 175
Thr Phe Pro Asn His Tyr Thr Ile Val Thr Gly Leu Tyr Pro Glu Ser
                        180 185 190
His Gly Ile Ile Asp Asn Asn Met Tyr Asp Val Asn Leu Asn Lys Asn
                195 200 205
Phe Ser Leu Ser Ser Lys Glu Gln Asn Asn Pro Ala Trp Trp His Gly
        210 215 220
Gln Pro Met Trp Leu Thr Ala Met Tyr Gln Gly Leu Lys Ala Ala Thr
225 230 235 240
Tyr Phe Trp Pro Gly Ser Glu Val Ala Ile Asn Gly Ser Phe Pro Ser
                                245 250 255
Ile Tyr Met Pro Tyr Asn Gly Ser Val Pro Phe Glu Glu Arg Ile Ser
                        260 265 270
Thr Leu Leu Lys Trp Leu Asp Leu Pro Lys Ala Glu Arg Pro Arg Phe
                275 280 285
Tyr Thr Met Tyr Phe Glu Glu Pro Asp Ser Ser Gly His Ala Gly Gly
        290 295 300
Pro Val Ser Ala Arg Val Ile Lys Ala Leu Gln Val Val Asp His Ala
305 310 315 320
Phe Gly Met Leu Met Glu Gly Leu Lys Gln Arg Asn Leu His Asn Cys
                                325 330 335
Val Asn Ile Ile Leu Leu Ala Asp His Gly Met Asp Gln Thr Tyr Cys
                        340 345 350
Asn Lys Met Glu Tyr Met Thr Asp Tyr Phe Pro Arg Ile Asn Phe Phe
                355 360 365
Tyr Met Tyr Glu Gly Pro Ala Pro Arg Ile Arg Ala His Asn Ile Pro
        370 375 380
His Asp Phe Phe Ser Phe Asn Ser Glu Glu Ile Val Arg Asn Leu Ser
385 390 395 400
Cys Arg Lys Pro Asp Gln His Phe Lys Pro Tyr Leu Thr Pro Asp Leu
                                405 410 415
Pro Lys Arg Leu His Tyr Ala Lys Asn Val Arg Ile Asp Lys Val His
                        420 425 430
Leu Phe Val Asp Gln Gln Trp Leu Ala Val Arg Ser Lys Ser Asn Thr
                435 440 445
Asn Cys Gly Gly Gly Asn His Gly Tyr Asn Asn Glu Phe Arg Ser Met
        450 455 460
Glu Ala Ile Phe Leu Ala His Gly Pro Ser Phe Lys Glu Lys Thr Glu
465 470 475 480
Val Glu Pro Phe Glu Asn Ile Glu Val Tyr Asn Leu Met Cys Asp Leu
                                485 490 495
Leu Arg Ile Gln Pro Ala Pro Asn Asn Gly Thr His Gly Ser Leu Asn
                        500 505 510
His Leu Leu Lys Val Pro Phe Tyr Glu Pro Ser His Ala Glu Glu Val
                515 520 525
Ser Lys Phe Ser Val Cys Gly Phe Ala Asn Pro Leu Pro Thr Glu Ser
        530 535 540
Leu Asp Cys Phe Cys Pro His Leu Gln Asn Ser Thr Gln Leu Glu Gln
545 550 555 560
Val Asn Gln Met Leu Asn Leu Thr Gln Glu Glu Ile Thr Ala Thr Val
                                565 570 575
Lys Val Asn Leu Pro Phe Gly Arg Pro Arg Val Leu Gln Lys Asn Val
                        580 585 590
Asp His Cys Leu Leu Tyr His Arg Glu Tyr Val Ser Gly Phe Gly Lys
                595 600 605
Ala Met Arg Met Pro Met Trp Ser Ser Tyr Thr Val Pro Gln Leu Gly
        610 615 620
Asp Thr Ser Pro Leu Pro Pro Thr Val Pro Asp Cys Leu Arg Ala Asp
625 630 635 640
Val Arg Val Pro Pro Ser Glu Ser Gln Lys Cys Ser Phe Tyr Leu Ala
                                645 650 655
Asp Lys Asn Ile Thr His Gly Phe Leu Tyr Pro Pro Ala Ser Asn Arg
                        660 665 670
Thr Ser Asp Ser Gln Tyr Asp Ala Leu Ile Thr Ser Asn Leu Val Pro
                675 680 685
Met Tyr Glu Glu Phe Arg Lys Met Trp Asp Tyr Phe His Ser Val Leu
        690 695 700
Leu Ile Lys His Ala Thr Glu Arg Asn Gly Val Asn Val Val Ser Gly
705 710 715 720
Pro Ile Phe Asp Tyr Asn Tyr Asp Gly His Phe Asp Ala Pro Asp Glu
                                725 730 735
Ile Thr Lys His Leu Ala Asn Thr Asp Val Pro Ile Pro Thr His Tyr
                        740 745 750
Phe Val Val Leu Thr Ser Cys Lys Asn Lys Ser His Thr Pro Glu Asn
                755 760 765
Cys Pro Gly Trp Leu Asp Val Leu Pro Phe Ile Ile Pro His Arg Pro
        770 775 780
Thr Asn Val Glu Ser Cys Pro Glu Gly Lys Pro Glu Ala Leu Trp Val
785 790 795 800
Glu Glu Arg Phe Thr Ala His Ile Ala Arg Val Arg Asp Val Glu Leu
                                805 810 815
Leu Thr Gly Leu Asp Phe Tyr Gln Asp Lys Val Gln Pro Val Ser Glu
                        820 825 830
Ile Leu Gln Leu Lys Thr Tyr Leu Pro Thr Phe Glu Thr Thr Ile Asp
                835 840 845
Lys Thr His Thr Cys Pro Pro Cys Pro Ala Pro Glu Leu Leu Gly Gly
        850 855 860
Pro Ser Val Phe Leu Phe Pro Pro Lys Pro Lys Asp Thr Leu Met Ile
865 870 875 880
Ser Arg Thr Pro Glu Val Thr Cys Val Val Val Asp Val Ser His Glu
                                885 890 895
Asp Pro Glu Val Lys Phe Asn Trp Tyr Val Asp Gly Val Glu Val His
                        900 905 910
Asn Ala Lys Thr Lys Pro Arg Glu Glu Gln Tyr Asn Ser Thr Tyr Arg
                915 920 925
Val Val Ser Val Leu Thr Val Leu His Gln Asp Trp Leu Asn Gly Lys
        930 935 940
Glu Tyr Lys Cys Lys Val Ser Asn Lys Ala Leu Pro Ala Pro Ile Glu
945 950 955 960
Lys Thr Ile Ser Lys Ala Lys Gly Gln Pro Arg Glu Pro Gln Val Tyr
                                965 970 975
Thr Leu Pro Pro Ser Arg Glu Glu Met Thr Lys Asn Gln Val Ser Leu
                        980 985 990
Thr Cys Leu Val Lys Gly Phe Tyr Pro Ser Asp Ile Ala Val Glu Trp
                995 1000 1005
Glu Ser Asn Gly Gln Pro Glu Asn Asn Tyr Lys Thr Thr Pro Pro
        1010 1015 1020
Val Leu Asp Ser Asp Gly Ser Phe Phe Leu Tyr Ser Lys Leu Thr
        1025 1030 1035
Val Asp Lys Ser Arg Trp Gln Gln Gly Asn Val Phe Ser Cys Ser
        1040 1045 1050
Val Met His Glu Ala Leu His Asn His Tyr Thr Gln Lys Ser Leu
        1055 1060 1065
Ser Leu Ser Pro Gly Lys
        1070
Singly underlined: signal peptide sequence; double-underlined:
beginning and end of NPP3; ** = cleavage position at the signal
peptide sequence; bold residues indicate albumin sequence
ENPP5-NPP3-Albumin sequence
SEQ. ID NO: 15
Met Thr Ser Lys Phe Leu Leu Val Ser Phe Ile Leu Ala Ala Leu Ser
1 5 10 15
Leu Ser Thr Thr Phe Ser**Lys Gln Gly Ser Cys Arg Lys Lys Cys Phe
                        20 25 30
Asp Ala Ser Phe Arg Gly Leu Glu Asn Cys Arg Cys Asp Val Ala Cys
                35 40 45
Lys Asp Arg Gly Asp Cys Cys Trp Asp Phe Glu Asp Thr Cys Val Glu
        50 55 60
Ser Thr Arg Ile Trp Met Cys Asn Lys Phe Arg Cys Gly Glu Arg Leu
65 70 75 80
Glu Ala Ser Leu Cys Ser Cys Ser Asp Asp Cys Leu Gln Arg Lys Asp
                                85 90 95
Cys Cys Ala Asp Tyr Lys Ser Val Cys Gln Gly Glu Thr Ser Trp Leu
                      100 105 110
Glu Glu Asn Cys Asp Thr Ala Gln Gln Ser Gln Cys Pro Glu Gly Phe
                115 120 125
Asp Leu Pro Pro Val Ile Leu Phe Ser Met Asp Gly Phe Arg Ala Glu
        130 135 140
Tyr Leu Tyr Thr Trp Asp Thr Leu Met Pro Asn Ile Asn Lys Leu Lys
145 150 155 160
Thr Cys Gly Ile His Ser Lys Tyr Met Arg Ala Met Tyr Pro Thr Lys
                                165 170 175
Thr Phe Pro Asn His Tyr Thr Ile Val Thr Gly Leu Tyr Pro Glu Ser
                        180 185 190
His Gly Ile Ile Asp Asn Asn Met Tyr Asp Val Asn Leu Asn Lys Asn
                195 200 205
Phe Ser Leu Ser Ser Lys Glu Gln Asn Asn Pro Ala Trp Trp His Gly
        210 215 220
Gln Pro Met Trp Leu Thr Ala Met Tyr Gln Gly Leu Lys Ala Ala Thr
225 230 235 240
Tyr Phe Trp Pro Gly Ser Glu Val Ala Ile Asn Gly Ser Phe Pro Ser
                                245 250 255
Ile Tyr Met Pro Tyr Asn Gly Ser Val Pro Phe Glu Glu Arg Ile Ser
                        260 265 270
Thr Leu Leu Lys Trp Leu Asp Leu Pro Lys Ala Glu Arg Pro Arg Phe
                275 280 285
Tyr Thr Met Tyr Phe Glu Glu Pro Asp Ser Ser Gly His Ala Gly Gly
        290 295 300
Pro Val Ser Ala Arg Val Ile Lys Ala Leu Gln Val Val Asp His Ala
305 310 315 320
Phe Gly Met Leu Met Glu Gly Leu Lys Gln Arg Asn Leu His Asn Cys
                                325 330 335
Val Asn Ile Ile Leu Leu Ala Asp His Gly Met Asp Gln Thr Tyr Cys
                        340 345 350
Asn Lys Met Glu Tyr Met Thr Asp Tyr Phe Pro Arg Ile Asn Phe Phe
                355 360 365
Tyr Met Tyr Glu Gly Pro Ala Pro Arg Ile Arg Ala His Asn Ile Pro
        370 375 380
His Asp Phe Phe Ser Phe Asn Ser Glu Glu Ile Val Arg Asn Leu Ser
385 390 395 400
Cys Arg Lys Pro Asp Gln His Phe Lys Pro Tyr Leu Thr Pro Asp Leu
                                405 410 415
Pro Lys Arg Leu His Tyr Ala Lys Asn Val Arg Ile Asp Lys Val His
                        420 425 430
Leu Phe Val Asp Gln Gln Trp Leu Ala Val Arg Ser Lys Ser Asn Thr
                435 440 445
Asn Cys Gly Gly Gly Asn His Gly Tyr Asn Asn Glu Phe Arg Ser Met
        450 455 460
Glu Ala Ile Phe Leu Ala His Gly Pro Ser Phe Lys Glu Lys Thr Glu
465 470 475 480
Val Glu Pro Phe Glu Asn Ile Glu Val Tyr Asn Leu Met Cys Asp Leu
                                485 490 495
Leu Arg Ile Gln Pro Ala Pro Asn Asn Gly Thr His Gly Ser Leu Asn
                        500 505 510
His Leu Leu Lys Val Pro Phe Tyr Glu Pro Ser His Ala Glu Glu Val
                515 520 525
Ser Lys Phe Ser Val Cys Gly Phe Ala Asn Pro Leu Pro Thr Glu Ser
        530 535 540
Leu Asp Cys Phe Cys Pro His Leu Gln Asn Ser Thr Gln Leu Glu Gln
545 550 555 560
Val Asn Gln Met Leu Asn Leu Thr Gln Glu Glu Ile Thr Ala Thr Val
                                565 570 575
Lys Val Asn Leu Pro Phe Gly Arg Pro Arg Val Leu Gln Lys Asn Val
                        580 585 590
Asp His Cys Leu Leu Tyr His Arg Glu Tyr Val Ser Gly Phe Gly Lys
                595 600 605
Ala Met Arg Met Pro Met Trp Ser Ser Tyr Thr Val Pro Gln Leu Gly
        610 615 620
Asp Thr Ser Pro Leu Pro Pro Thr Val Pro Asp Cys Leu Arg Ala Asp
625 630 635 640
Val Arg Val Pro Pro Ser Glu Ser Gln Lys Cys Ser Phe Tyr Leu Ala
                                645 650 655
Asp Lys Asn Ile Thr His Gly Phe Leu Tyr Pro Pro Ala Ser Asn Arg
                        660 665 670
Thr Ser Asp Ser Gln Tyr Asp Ala Leu Ile Thr Ser Asn Leu Val Pro
                675 680 685
Met Tyr Glu Glu Phe Arg Lys Met Trp Asp Tyr Phe His Ser Val Leu
        690 695 700
Leu Ile Lys His Ala Thr Glu Arg Asn Gly Val Asn Val Val Ser Gly
705 710 715 720
Pro Ile Phe Asp Tyr Asn Tyr Asp Gly His Phe Asp Ala Pro Asp Glu
                                725 730 735
Ile Thr Lys His Leu Ala Asn Thr Asp Val Pro Ile Pro Thr His Tyr
                        740 745 750
Phe Val Val Leu Thr Ser Cys Lys Asn Lys Ser His Thr Pro Glu Asn
                755 760 765
Cys Pro Gly Trp Leu Asp Val Leu Pro Phe Ile Ile Pro His Arg Pro
        770 775 780
Thr Asn Val Glu Ser Cys Pro Glu Gly Lys Pro Glu Ala Leu Trp Val
785 790 795 800
Glu Glu Arg Phe Thr Ala His Ile Ala Arg Val Arg Asp Val Glu Leu
                                805 810 815
Leu Thr Gly Leu Asp Phe Tyr Gln Asp Lys Val Gln Pro Val Ser Glu
                        820 825 830
Ile Leu Gln Leu Lys Thr Tyr Leu Pro Thr Phe Glu Thr Thr Ile Gly
                835 840 845
Gly Gly Ser Gly Gly Gly Gly Ser Gly Gly Gly Gly Ser Met Lys Trp
        850 855 860
Val Thr Phe Leu Leu Leu Leu Phe Val Ser Gly Ser Ala Phe Ser Arg
865 870 875 880
Gly Val Phe Arg Arg Glu Ala His Lys Ser Glu Ile Ala His Arg Tyr
                                885 890 895
Asn Asp Leu Gly Glu Gln His Phe Lys Gly Leu Val Leu Ile Ala Phe
                        900 905 910
Ser Gln Tyr Leu Gln Lys Cys Ser Tyr Asp Glu His Ala Lys Leu Val
                915 920 925
Gln Glu Val Thr Asp Phe Ala Lys Thr Cys Val Ala Asp Glu Ser Ala
        930 935 940
Ala Asn Cys Asp Lys Ser Leu His Thr Leu Phe Gly Asp Lys Leu Cys
945 950 955 960
Ala Ile Pro Asn Leu Arg Glu Asn Tyr Gly Glu Leu Ala Asp Cys Cys
                                965 970 975
Thr Lys Gln Glu Pro Glu Arg Asn Glu Cys Phe Leu Gln His Lys Asp
                        980 985 990
Asp Asn Pro Ser Leu Pro Pro Phe Glu Arg Pro Glu Ala Glu Ala Met
                995 1000 1005
Cys Thr Ser Phe Lys Glu Asn Pro Thr Thr Phe Met Gly His Tyr
        1010 1015 1020
Leu His Glu Val Ala Arg Arg His Pro Tyr Phe Tyr Ala Pro Glu
        1025 1030 1035
Leu Leu Tyr Tyr Ala Glu Gln Tyr Asn Glu Ile Leu Thr Gln Cys
        1040 1045 1050
Cys Ala Glu Ala Asp Lys Glu Ser Cys Leu Thr Pro Lys Leu Asp
        1055 1060 1065
Gly Val Lys Glu Lys Ala Leu Val Ser Ser Val Arg Gln Arg Met
        1070 1075 1080
Lys Cys Ser Ser Met Gln Lys Phe Gly Glu Arg Ala Phe Lys Ala
        1085 1090 1095
Trp Ala Val Ala Arg Leu Ser Gln Thr Phe Pro Asn Ala Asp Phe
        1100 1105 1110
Ala Glu Ile Thr Lys Leu Ala Thr Asp Leu Thr Lys Val Asn Lys
        1115 1120 1125
Glu Cys Cys His Gly Asp Leu Leu Glu Cys Ala Asp Asp Arg Ala
        1130 1135 1140
Glu Leu Ala Lys Tyr Met Cys Glu Asn Gln Ala Thr Ile Ser Ser
        1145 1150 1155
Lys Leu Gln Thr Cys Cys Asp Lys Pro Leu Leu Lys Lys Ala His
        1160 1165 1170
Cys Leu Ser Glu Val Glu His Asp Thr Met Pro Ala Asp Leu Pro
        1175 1180 1185
Ala Ile Ala Ala Asp Phe Val Glu Asp Gln Glu Val Cys Lys Asn
        1190 1195 1200
Tyr Ala Glu Ala Lys Asp Val Phe Leu Gly Thr Phe Leu Tyr Glu
        1205 1210 1215
Tyr Ser Arg Arg His Pro Asp Tyr Ser Val Ser Leu Leu Leu Arg
        1220 1225 1230
Leu Ala Lys Lys Tyr Glu Ala Thr Leu Glu Lys Cys Cys Ala Glu
        1235 1240 1245
Ala Asn Pro Pro Ala Cys Tyr Gly Thr Val Leu Ala Glu Phe Gln
        1250 1255 1260
Pro Leu Val Glu Glu Pro Lys Asn Leu Val Lys Thr Asn Cys Asp
        1265 1270 1275
Leu Tyr Glu Lys Leu Gly Glu Tyr Gly Phe Gln Asn Ala Ile Leu
        1280 1285 1290
Val Arg Tyr Thr Gln Lys Ala Pro Gln Val Ser Thr Pro Thr Leu
        1295 1300 1305
Val Glu Ala Ala Arg Asn Leu Gly Arg Val Gly Thr Lys Cys Cys
        1310 1315 1320
Thr Leu Pro Glu Asp Gln Arg Leu Pro Cys Val Glu Asp Tyr Leu
        1325 1330 1335
Ser Ala Ile Leu Asn Arg Val Cys Leu Leu His Glu Lys Thr Pro
        1340 1345 1350
Val Ser Glu His Val Thr Lys Cys Cys Ser Gly Ser Leu Val Glu
        1355 1360 1365
Arg Arg Pro Cys Phe Ser Ala Leu Thr Val Asp Glu Thr Tyr Val
        1370 1375 1380
Pro Lys Glu Phe Lys Ala Glu Thr Phe Thr Phe His Ser Asp Ile
        1385 1390 1395
Cys Thr Leu Pro Glu Lys Glu Lys Gln Ile Lys Lys Gln Thr Ala
        1400 1405 1410
Leu Ala Glu Leu Val Lys His Lys Pro Lys Ala Thr Ala Glu Gln
        1415 1420 1425
Leu Lys Thr Val Met Asp Asp Phe Ala Gln Phe Leu Asp Thr Cys
        1430 1435 1440
Cys Lys Ala Ala Asp Lys Asp Thr Cys Phe Ser Thr Glu Gly Pro
        1445 1450 1455
Asn Leu Val Thr Arg Cys Lys Asp Ala Leu Ala
        1460 1465
Singly underlined: signal peptide sequence; double-underlined:
beginning and end of NPP3;
** = cleavage position at the signal
peptide sequence; bold residues indicate albumin sequence
ENPP5 Protein Export Signal Sequence
SEQ. ID NO: 16
Met Thr Ser Lys Phe Leu Leu Val Ser Phe Ile Leu Ala Ala Leu Ser
1 5 10 15
Leu Ser Thr Thr Phe Ser Xaa
20
SEQ. ID NO: 17
ENPP51-Fc
Met Thr Ser Lys Phe Leu Leu Val Ser Phe Ile Leu Ala Ala Leu Ser
1 5 10 15
Leu Ser Thr Thr Phe Ser**Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                      100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
180 190 185
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
                675 680 685
Ile Val Pro Met Tyr Gln Ser Phe Gln Val Ile Trp Arg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp Thr Leu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp Asp Lys Thr His Thr Cys Pro Pro Cys Pro Ala Pro
        850 855 860
Glu Leu Leu Gly Gly Pro Ser Val Phe Leu Phe Pro Pro Lys Pro Lys
865 870 875 880
Asp Thr Leu Met Ile Ser Arg Thr Pro Glu Val Thr Cys Val Val Val
                                885 890 895
Asp Val Ser His Glu Asp Pro Glu Val Lys Phe Asn Trp Tyr Val Asp
                        900 905 910
Gly Val Glu Val His Asn Ala Lys Thr Lys Pro Arg Glu Glu Gln Tyr
                915 920 925
Asn Ser Thr Tyr Arg Val Val Ser Val Leu Thr Val Leu His Gln Asp
        930 935 940
Trp Leu Asn Gly Lys Glu Tyr Lys Cys Lys Val Ser Asn Lys Ala Leu
945 950 955 960
Pro Ala Pro Ile Glu Lys Thr Ile Ser Lys Ala Lys Gly Gln Pro Arg
                                965 970 975
Glu Pro Gln Val Tyr Thr Leu Pro Pro Ser Arg Glu Glu Met Thr Lys
                        980 985 990
Asn Gln Val Ser Leu Thr Cys Leu Val Lys Gly Phe Tyr Pro Ser Asp
                995 1000 1005
Ile Ala Val Glu Trp Glu Ser Asn Gly Gln Pro Glu Asn Asn Tyr
        1010 1015 1020
Lys Thr Thr Pro Pro Val Leu Asp Ser Asp Gly Ser Phe Phe Leu
        1025 1030 1035
Tyr Ser Lys Leu Thr Val Asp Lys Ser Arg Trp Gln Gln Gly Asn
        1040 1045 1050
His Glu Ala Leu His Asn His Tyr Val Phe Ser Cys Ser Val Met
        1055 1060 1065
Thr Gln Lys Ser Leu Ser Leu Ser Pro Gly Lys
        1070 1075
Singly underlined: signal peptide sequence; double-underlined:
beginning and end of NPP1; ** = cleavage position at the signal
peptide sequence; bold residues indicate Fc sequence
ENPP71-Fc Amino Acid Sequence
SEQ. ID NO: 18
Met Arg Gly Pro Ala Val Leu Leu Thr Val Ala Leu Ala Thr Leu Leu
1 5 10 15
Ala Pro Gly Ala Gly Ala** Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                      100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
                        180 185 190
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
                675 680 685
Ile Val Pro Met Tyr Gln Ser Phe Gln Val Ile Trp Arg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp Thr Leu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp Leu Ile Asn Asp Lys Thr His Thr Cys Pro Pro Cys
        850 855 860
Pro Ala Pro Glu Leu Leu Gly Gly Pro Ser Val Phe Leu Phe Pro Pro
865 870 875 880
Lys Pro Lys Asp Thr Leu Met Ile Ser Arg Thr Pro Glu Val Thr Cys
                                885 890 895
Val Val Val Asp Val Ser His Glu Asp Pro Glu Val Lys Phe Asn Trp
                        900 905 910
Tyr Val Asp Gly Val Glu Val His Asn Ala Lys Thr Lys Pro Arg Glu
                915 920 925
Glu Gln Tyr Asn Ser Thr Tyr Arg Val Val Ser Val Leu Thr Val Leu
        930 935 940
His Gln Asp Trp Leu Asn Gly Lys Glu Tyr Lys Cys Lys Val Ser Asn
945 950 955 960
Lys Ala Leu Pro Ala Pro Ile Glu Lys Thr Ile Ser Lys Ala Lys Gly
                                965 970 975
Gln Pro Arg Glu Pro Gln Val Tyr Thr Leu Pro Pro Ser Arg Glu Glu
                        980 985 990
Met Thr Lys Asn Gln Val Ser Leu Thr Cys Leu Val Lys Gly Phe Tyr
                995 1000 1005
Pro Ser Asp Ile Ala Val Glu Trp Glu Ser Asn Gly Gln Pro Glu
        1010 1015 1020
Asn Asn Tyr Lys Thr Thr Pro Pro Val Leu Asp Ser Asp Gly Ser
        1025 1030 1035
Phe Phe Leu Tyr Ser Lys Leu Thr Val Asp Lys Ser Arg Trp Gln
        1040 1045 1050
Gln Gly Asn Val Phe Ser Cys Ser Val Met His Glu Ala Leu His
        1055 1060 1065
Asn His Tyr Thr Gln Lys Ser Leu Ser Leu Ser Pro Gly Lys
        1070 1075 1080
Singly underlined: signal peptide sequence; double-underlined:
beginning and end of NPP1; ** = cleavage position at the signal
peptide sequence; bold residues indicate Fc sequence
ENPP71 (lacking NPP1 N-Terminus GLK) Amino Acid Sequence :
SEQ. ID NO: 19
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                      100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Asn Leu Ile Leu Ile Ser Asp His Gly Met Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe Ser Gln Glu
                835 840 845
Asp
Singly underlined: signal peptide sequence; double-underlined:
beginning and end of NPP1; ** = cleavage position at the signal
peptide sequence
ENPP71 (lacking NPP1 N-Terminus GLK)-Fc Amino
Acid Sequence:
SEQ. ID NO: 20
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                      100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Asn Leu Ile Leu Ile Ser Asp His Gly Met Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe Ser Gln Glu
                835 840 845
Asp Leu Ile Asn Asp Lys Thr His Thr Cys Pro Pro Cys Pro Ala Pro
        850 855 860
Glu Leu Leu Gly Gly Pro Ser Val Phe Leu Phe Pro Pro Lys Pro Lys
865 870 875 880
Asp Thr Leu Met Ile Ser Arg Thr Pro Glu Val Thr Cys Val Val Val
                                885 890 895
Asp Val Ser His Glu Asp Pro Glu Val Lys Phe Asn Trp Tyr Val Asp
                        900 905 910
Gly Val Glu Val His Asn Ala Lys Thr Lys Pro Arg Glu Glu Gln Tyr
                915 920 925
Asn Ser Thr Tyr Arg Val Val Ser Val Leu Thr Val Leu His Gln Asp
        930 935 940
Trp Leu Asn Gly Lys Glu Tyr Lys Cys Lys Val Ser Asn Lys Ala Leu
945 950 955 960
Pro Ala Pro Ile Glu Lys Thr Ile Ser Lys Ala Lys Gly Gln Pro Arg
                                965 970 975
Glu Pro Gln Val Tyr Thr Leu Pro Pro Ser Arg Glu Glu Met Thr Lys
                        980 985 990
Asn Gln Val Ser Leu Thr Cys Leu Val Lys Gly Phe Tyr Pro Ser Asp
                995 1000 1005
Ile Ala Val Glu Trp Glu Ser Asn Gly Gln Pro Glu Asn Asn Tyr
        1010 1015 1020
Lys Thr Thr Pro Pro Val Leu Asp Ser Asp Gly Ser Phe Phe Leu
        1025 1030 1035
Tyr Ser Lys Leu Thr Val Asp Lys Ser Arg Trp Gln Gln Gly Asn
        1040 1045 1050
Val Phe Ser Cys Ser Val Met His Glu Ala Leu His Asn His Tyr
        1055 1060 1065
Thr Gln Lys Ser Leu Ser Leu Ser Pro Gly Lys
        1075 1070
Singly underlined: signal peptide sequence; double-underlined:
beginning and end of NPP1; ** = cleavage position at the signal
peptide sequence; bold residues indicate Fc sequence
ENPP71 (lacking NPP1 N-Terminus GLK)-ALB Amino
Acid Sequence
SEQ. ID NO: 21
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                      100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Leu Ile Asn Ile Leu Asp His Ser Met Gly Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe Ser Gln Glu
                835 840 845
Asp Arg Ser Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1240 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355 1360 1365
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu Pro Glu
        1385 1390 1395
Lys Glu Lys Gln Ile Lys Lys Gln Thr Ala Leu Ala Glu Leu Val
        1400 1405 1410
Lys His Lys Pro Lys Ala Thr Ala Glu Gln Leu Lys Thr Val Met
        1415 1420 1425
Asp Asp Phe Ala Gln Phe Leu Asp Thr Cys Cys Lys Ala Ala Asp
        1430 1435 1440
Lys Asp Thr Cys Phe Ser Thr Glu Gly Pro Asn Leu Val Thr Arg
        1445 1450 1455
Cys Lys Asp Ala Leu Ala Arg Ser Trp Ser His Pro Gln Phe Glu
        1460 1465 1470
Lys
Singly underlined: signal peptide sequence; double-underlined:
beginning and end of NPP1; ** = cleavage position at the signal
peptide sequence; bold residues indicate albumin sequence
ENPP7-NPP3-Fc sequence:
SEQ. ID NO: 22
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                      100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Tyr Thr Ile Val Thr Gly Leu Tyr Pro Glu Ser His Gly
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Asp Lys Thr
                835 840 845
His Thr Cys Pro Pro Cys Pro Ala Pro Glu Leu Leu Gly Gly Pro Ser
        850 855 860
Val Phe Leu Phe Pro Pro Lys Pro Lys Asp Thr Leu Met Ile Ser Arg
865 870 875 880
Thr Pro Glu Val Thr Cys Val Val Val Asp Val Ser His Glu Asp Pro
                                885 890 895
Glu Val Lys Phe Asn Trp Tyr Val Asp Gly Val Glu Val His Asn Ala
                        900 905 910
Lys Thr Lys Pro Arg Glu Glu Gln Tyr Asn Ser Thr Tyr Arg Val Val
                915 920 925
Ser Val Leu Thr Val Leu His Gln Asp Trp Leu Asn Gly Lys Glu Tyr
        930 935 940
Lys Cys Lys Val Ser Asn Lys Ala Leu Pro Ala Pro Ile Glu Lys Thr
945 950 955 960
Ile Ser Lys Ala Lys Gly Gln Pro Arg Glu Pro Gln Val Tyr Thr Leu
                                965 970 975
Pro Pro Ser Arg Glu Glu Met Thr Lys Asn Gln Val Ser Leu Thr Cys
                        980 985 990
Leu Val Lys Gly Phe Tyr Pro Ser Asp Ile Ala Val Glu Trp Glu Ser
                995 1000 1005
Asn Gly Gln Pro Glu Asn Asn Tyr Lys Thr Thr Pro Pro Val Leu
        1010 1015 1020
Asp Ser Asp Gly Ser Phe Phe Leu Tyr Ser Lys Leu Thr Val Asp
        1025 1030 1035
Lys Ser Arg Trp Gln Gln Gly Asn Val Phe Ser Cys Ser Val Met
        1040 1045 1050
His Glu Ala Leu His Asn His Ser Pro Gly Lys Tyr Thr Gln Lys
        1055 1060 1065
Ser Leu Ser Leu
        1070
Singly underlined: signal peptide sequence; double-underlined:
beginning and end of NPP3; ** = cleavage position at the signal
peptide sequence; bold residues indicate Fc sequence
ENPP71-Albumin
SEQ. ID NO: 23
Met Arg Gly Pro Ala Val Leu Leu Thr Val Ala Leu Ala Thr Leu Leu
1 5 10 15
Ala Pro Gly Ala Gly Leu Lys**Pro Ser Cys Ala Lys Glu Val Lys Ser
                        20 25 30
Cys Lys Gly Arg Cys Phe Glu Arg Thr Phe Gly Asn Cys Arg Cys Asp
                35 40 45
Ala Ala Cys Val Glu Leu Gly Asn Cys Cys Leu Asp Tyr Gln Glu Thr
        50 55 60
Cys Ile Glu Pro Glu His Ile Trp Thr Cys Asn Lys Phe Arg Cys Gly
65 70 75 80
Glu Lys Arg Leu Thr Arg Ser Leu Cys Ala Cys Ser Asp Asp Cys Lys
                                85 90 95
Asp Lys Gly Asp Cys Cys Ile Asn Tyr Ser Ser Val Cys Gln Gly Glu
                      100 105 110
Lys Ser Trp Val Glu Glu Pro Cys Glu Ser Ile Asn Glu Pro Gln Cys
                115 120 125
Pro Ala Gly Phe Glu Thr Pro Pro Thr Leu Leu Phe Ser Leu Asp Gly
        130 135 140
Phe Arg Ala Glu Tyr Leu His Thr Trp Gly Gly Leu Leu Pro Val Ile
145 150 155 160
Ser Lys Leu Lys Lys Cys Gly Thr Tyr Thr Lys Asn Met Arg Pro Val
                                165 170 175
Tyr Pro Thr Lys Thr Phe Pro Asn His Tyr Ser Ile Val Thr Gly Leu
                        180 185 190
Tyr Pro Glu Ser His Gly Ile Ile Asp Asn Lys Met Tyr Asp Pro Lys
                195 200 205
Met Asn Ala Ser Phe Ser Leu Lys Ser Lys Glu Lys Phe Asn Pro Glu
        210 215 220
Trp Tyr Lys Gly Glu Pro Ile Trp Val Thr Ala Lys Tyr Gln Gly Leu
225 230 235 240
Lys Ser Gly Thr Phe Phe Trp Pro Gly Ser Asp Val Glu Ile Asn Gly
                                245 250 255
Ile Phe Pro Asp Ile Tyr Lys Met Tyr Asn Gly Ser Val Pro Phe Glu
                        260 265 270
Glu Arg Ile Leu Ala Val Leu Gln Trp Leu Gln Leu Pro Lys Asp Glu
                275 280 285
Arg Pro His Phe Tyr Thr Leu Tyr Leu Glu Glu Pro Asp Ser Ser Gly
        290 295 300
His Ser Tyr Gly Pro Val Ser Ser Glu Val Ile Lys Ala Leu Gln Arg
305 310 315 320
Val Asp Gly Met Val Gly Met Leu Met Asp Gly Leu Lys Glu Leu Asn
                                325 330 335
Leu His Arg Cys Leu Asn Leu Ile Leu Ile Ser Asp His Gly Met Glu
                        340 345 350
Gln Gly Ser Cys Lys Lys Tyr Ile Tyr Leu Asn Lys Tyr Leu Gly Asp
                355 360 365
Val Lys Asn Ile Lys Val Ile Tyr Gly Pro Ala Ala Arg Leu Arg Pro
        370 375 380
Ser Asp Val Pro Asp Lys Tyr Tyr Ser Phe Asn Tyr Glu Gly Ile Ala
385 390 395 400
Arg Asn Leu Ser Cys Arg Glu Pro Asn Gln His Phe Lys Pro Tyr Leu
                                405 410 415
Lys His Phe Leu Pro Lys Arg Leu His Phe Ala Lys Ser Asp Arg Ile
                        420 425 430
Glu Pro Leu Thr Phe Tyr Leu Asp Pro Gln Trp Gln Leu Ala Leu Asn
                435 440 445
Pro Ser Glu Arg Lys Tyr Cys Gly Ser Gly Phe His Gly Ser Asp Asn
        450 455 460
Val Phe Ser Asn Met Gln Ala Leu Phe Val Gly Tyr Gly Pro Gly Phe
465 470 475 480
Lys His Gly Ile Glu Ala Asp Thr Phe Glu Asn Ile Glu Val Tyr Asn
                                485 490 495
Leu Met Cys Asp Leu Leu Asn Leu Thr Pro Ala Pro Asn Asn Gly Thr
                        500 505 510
His Gly Ser Leu Asn His Leu Leu Lys Asn Pro Val Tyr Thr Pro Lys
                515 520 525
His Pro Lys Glu Val His Pro Leu Val Gln Cys Pro Phe Thr Arg Asn
        530 535 540
Pro Arg Asp Asn Leu Gly Cys Ser Cys Asn Pro Ser Ile Leu Pro Ile
545 550 555 560
Glu Asp Phe Gln Thr Gln Phe Asn Leu Thr Val Ala Glu Glu Lys Ile
                                565 570 575
Ile Lys His Glu Thr Leu Pro Tyr Gly Arg Pro Arg Val Leu Gln Lys
                        580 585 590
Glu Asn Thr Ile Cys Leu Leu Ser Gln His Gln Phe Met Ser Gly Tyr
                595 600 605
Ser Gln Asp Ile Leu Met Pro Leu Trp Thr Ser Tyr Thr Val Asp Arg
        610 615 620
Asn Asp Ser Phe Ser Thr Glu Asp Phe Ser Asn Cys Leu Tyr Gln Asp
625 630 635 640
Phe Arg Ile Pro Leu Ser Pro Val His Lys Cys Ser Phe Tyr Lys Asn
                                645 650 655
Asn Thr Lys Val Ser Tyr Gly Phe Leu Ser Pro Pro Gln Leu Asn Lys
                        660 665 670
Asn Ser Ser Gly Ile Tyr Ser Glu Ala Leu Leu Thr Thr Asn Ile Val
                675 680 685
Pro Met Tyr Gln Ser Phe Gln Val Ile Trp Arg Tyr Phe His Asp Thr
        690 695 700
Leu Leu Arg Lys Tyr Ala Glu Glu Arg Asn Gly Val Asn Val Val Ser
705 710 715 720
Gly Pro Val Phe Asp Phe Asp Tyr Asp Gly Arg Cys Asp Ser Leu Glu
                                725 730 735
Asn Leu Arg Gln Lys Arg Arg Val Ile Arg Asn Gln Glu Ile Leu Ile
                        740 745 750
Pro Thr His Phe Phe Ile Val Leu Thr Ser Cys Lys Asp Thr Ser Gln
                755 760 765
Thr Pro Leu His Cys Glu Asn Leu Asp Thr Leu Ala Phe Ile Leu Pro
        770 775 780
His Arg Thr Asp Asn Ser Glu Ser Cys Val His Gly Lys His Asp Ser
785 790 795 800
Ser Trp Val Glu Glu Leu Leu Met Leu His Arg Ala Arg Ile Thr Asp
                                805 810 815
Val Glu His Ile Thr Gly Leu Ser Phe Tyr Gln Gln Arg Lys Glu Pro
                        820 825 830
Val Ser Asp Ile Leu Lys Leu Lys Thr His Leu Pro Thr Phe Ser Gln
                835 840 845
Glu Asp Gly Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1240 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355 1360 1365
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu
        1385 1390 1395
Singly underlined: signal peptide sequence; double-underlined:
beginning and end of NPP3; ** = cleavage position at the signal
peptide sequence; bold residues indicate Fc sequence
ENPP7-NPP3-Albumin
SEQ. ID NO: 24
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                      100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Thr Ile Val Thr Leu Tyr Pro Glu His Gly Tyr Gly Ser
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Gly Gly Gly
                835 840 845
Ser Gly Gly Gly Gly Ser Gly Gly Gly Gly Ser Met Lys Trp Val Thr
        850 855 860
Phe Leu Leu Leu Leu Phe Val Ser Gly Ser Ala Phe Ser Arg Gly Val
865 870 875 880
Phe Arg Arg Glu Ala His Lys Ser Glu Ile Ala His Arg Tyr Asn Asp
                                885 890 895
Leu Gly Glu Gln His Phe Lys Gly Leu Val Leu Ile Ala Phe Ser Gln
                        900 905 910
Tyr Leu Gln Lys Cys Ser Tyr Asp Glu His Ala Lys Leu Val Gln Glu
                915 920 925
Val Thr Asp Phe Ala Lys Thr Cys Val Ala Asp Glu Ser Ala Ala Asn
        930 935 940
Cys Asp Lys Ser Leu His Thr Leu Phe Gly Asp Lys Leu Cys Ala Ile
945 950 955 960
Pro Asn Leu Arg Glu Asn Tyr Gly Glu Leu Ala Asp Cys Cys Thr Lys
                                965 970 975
Gln Glu Pro Glu Arg Asn Glu Cys Phe Leu Gln His Lys Asp Asp Asn
                        980 985 990
Pro Ser Leu Pro Pro Phe Glu Arg Pro Glu Ala Glu Ala Met Cys Thr
                995 1000 1005
Ser Phe Lys Glu Asn Pro Thr Thr Phe Met Gly His Tyr Leu His
        1010 1015 1020
Glu Val Ala Arg Arg His Pro Tyr Phe Tyr Ala Pro Glu Leu Leu
        1025 1030 1035
Tyr Tyr Ala Glu Gln Tyr Asn Glu Ile Leu Thr Gln Cys Cys Ala
        1040 1045 1050
Glu Ala Asp Lys Glu Ser Cys Leu Thr Pro Lys Leu Asp Gly Val
        1055 1060 1065
Lys Glu Lys Ala Leu Val Ser Ser Val Arg Gln Arg Met Lys Cys
        1070 1075 1080
Ser Ser Met Gln Lys Phe Gly Glu Arg Ala Phe Lys Ala Trp Ala
        1085 1090 1095
Val Ala Arg Leu Ser Gln Thr Phe Pro Asn Ala Asp Phe Ala Glu
        1100 1105 1110
Ile Thr Lys Leu Ala Thr Asp Leu Thr Lys Val Asn Lys Glu Cys
        1115 1120 1125
Cys His Gly Asp Leu Leu Glu Cys Ala Asp Asp Arg Ala Glu Leu
        1130 1135 1140
Ala Lys Tyr Met Cys Glu Asn Gln Ala Thr Ile Ser Ser Lys Leu
        1145 1150 1155
Gln Thr Cys Cys Asp Lys Pro Leu Leu Lys Lys Ala His Cys Leu
        1160 1165 1170
Ser Glu Val Glu His Asp Thr Met Pro Ala Asp Leu Pro Ala Ile
        1175 1180 1185
Ala Ala Asp Phe Val Glu Asp Gln Glu Val Cys Lys Asn Tyr Ala
        1190 1195 1200
Glu Ala Lys Asp Val Phe Leu Gly Thr Phe Leu Tyr Glu Tyr Ser
        1205 1210 1215
Arg Arg His Pro Asp Tyr Ser Val Ser Leu Leu Leu Arg Leu Ala
        1220 1225 1230
Lys Lys Tyr Glu Ala Thr Leu Glu Lys Cys Cys Ala Glu Ala Asn
        1235 1240 1245
Pro Pro Ala Cys Tyr Gly Thr Val Leu Ala Glu Phe Gln Pro Leu
        1250 1255 1260
Val Glu Glu Pro Lys Asn Leu Val Lys Thr Asn Cys Asp Leu Tyr
        1265 1270 1275
Glu Lys Leu Gly Glu Tyr Gly Phe Gln Asn Ala Ile Leu Val Arg
        1280 1285 1290
Tyr Thr Gln Lys Ala Pro Gln Val Ser Thr Pro Thr Leu Val Glu
        1295 1300 1305
Ala Ala Arg Asn Leu Gly Arg Val Gly Thr Lys Cys Cys Thr Leu
        1310 1315 1320
Pro Glu Asp Gln Arg Leu Pro Cys Val Glu Asp Tyr Leu Ser Ala
        1325 1330 1335
Ile Leu Asn Arg Val Cys Leu Leu His Glu Lys Thr Pro Val Ser
        1340 1345 1350
Glu His Val Thr Lys Cys Cys Ser Gly Ser Leu Val Glu Arg Arg
        1355 1360 1365
Pro Cys Phe Ser Ala Leu Thr Val Asp Glu Thr Tyr Val Pro Lys
        1370 1375 1380
Glu Phe Lys Ala Glu Thr Phe Thr Phe His Ser Asp Ile Cys Thr
        1385 1390 1395
Leu Pro Glu Lys Glu Lys Gln Ile Lys Lys Gln Thr Ala Leu Ala
        1400 1405 1410
Glu Leu Val Lys His Lys Pro Lys Ala Thr Ala Glu Gln Leu Lys
        1415 1420 1425
Thr Val Met Asp Asp Phe Ala Gln Phe Leu Asp Thr Cys Cys Lys
        1430 1435 1440
Ala Ala Asp Lys Asp Thr Cys Phe Ser Thr Glu Gly Pro Asn Leu
        1445 1450 1455
Val Thr Arg Cys Lys Asp Ala Leu Ala
        1460 1465
Singly underlined: signal peptide sequence; double-underlined:
beginning and end of NPP3; ** = cleavage position at the signal
peptide sequence; bold residues indicate albumin sequence
ENPP7-ENPP3-Albumin
SEQ. ID NO: 25
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                      100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Tyr Thr Ile Val Thr Gly Leu Tyr Pro Glu Ser His Gly
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Asp Lys Thr
                835 840 845
His Thr Cys Pro Pro Cys Pro Ala Pro Glu Leu Leu Gly Gly Pro Ser
        850 855 860
Val Phe Leu Phe Pro Pro Lys Pro Lys Asp Thr Leu Met Ile Ser Arg
865 870 875 880
Thr Pro Glu Val Thr Gly Gly Gly Ser Gly Gly Gly Gly Ser Gly Gly
                                885 890 895
Gly Gly Ser Met Lys Trp Val Thr Phe Leu Leu Leu Leu Phe Val Ser
                        900 905 910
Gly Ser Ala Phe Ser Arg Gly Val Phe Arg Arg Glu Ala His Lys Ser
                915 920 925
Glu Ile Ala His Arg Tyr Asn Asp Leu Gly Glu Gln His Phe Lys Gly
        930 935 940
Leu Val Leu Ile Ala Phe Ser Gln Tyr Leu Gln Lys Cys Ser Tyr Asp
945 950 955 960
Glu His Ala Lys Leu Val Gln Glu Val Thr Asp Phe Ala Lys Thr Cys
                                965 970 975
Val Ala Asp Glu Ser Ala Ala Asn Cys Asp Lys Ser Leu His Thr Leu
                        980 985 990
Phe Gly Asp Lys Leu Cys Ala Ile Pro Asn Leu Arg Glu Asn Tyr Gly
                995 1000 1005
Glu Leu Ala Asp Cys Cys Thr Lys Gln Glu Pro Glu Arg Asn Glu
        1010 1015 1020
Cys Phe Leu Gln His Lys Asp Asp Asn Pro Ser Leu Pro Pro Phe
        1025 1030 1035
Glu Arg Pro Glu Ala Glu Ala Met Cys Thr Ser Phe Lys Glu Asn
        1040 1045 1050
Pro Thr Thr Phe Met Gly His Tyr Leu His Glu Val Ala Arg Arg
        1055 1060 1065
His Pro Tyr Phe Tyr Ala Pro Glu Leu Leu Tyr Tyr Ala Glu Gln
        1070 1075 1080
Tyr Asn Glu Ile Leu Thr Gln Cys Cys Ala Glu Ala Asp Lys Glu
        1085 1090 1095
Ser Cys Leu Thr Pro Lys Leu Asp Gly Val Lys Glu Lys Ala Leu
        1100 1105 1110
Val Ser Ser Val Arg Gln Arg Met Lys Cys Ser Ser Met Gln Lys
        1115 1120 1125
Phe Gly Glu Arg Ala Phe Lys Ala Trp Ala Val Ala Arg Leu Ser
        1130 1135 1140
Gln Thr Phe Pro Asn Ala Asp Phe Ala Glu Ile Thr Lys Leu Ala
        1145 1150 1155
Thr Asp Leu Thr Lys Val Asn Lys Glu Cys Cys His Gly Asp Leu
        1160 1165 1170
Leu Glu Cys Ala Asp Asp Arg Ala Glu Leu Ala Lys Tyr Met Cys
        1175 1180 1185
Glu Asn Gln Ala Thr Ile Ser Ser Lys Leu Gln Thr Cys Cys Asp
        1190 1195 1200
Lys Pro Leu Leu Lys Lys Ala His Cys Leu Ser Glu Val Glu His
        1205 1210 1215
Asp Thr Met Pro Ala Asp Leu Pro Ala Ile Ala Ala Asp Phe Val
        1220 1225 1230
Glu Asp Gln Glu Val Cys Lys Asn Tyr Ala Glu Ala Lys Asp Val
        1235 1240 1245
Phe Leu Gly Thr Phe Leu Tyr Glu Tyr Ser Arg Arg His Pro Asp
        1250 1255 1260
Tyr Ser Val Ser Leu Leu Leu Arg Leu Ala Lys Lys Tyr Glu Ala
        1265 1270 1275
Thr Leu Glu Lys Cys Cys Ala Glu Ala Asn Pro Pro Ala Cys Tyr
        1280 1285 1290
Gly Thr Val Leu Ala Glu Phe Gln Pro Leu Val Glu Glu Pro Lys
        1295 1300 1305
Asn Leu Val Lys Thr Asn Cys Asp Leu Tyr Glu Lys Leu Gly Glu
        1310 1315 1320
Tyr Gly Phe Gln Asn Ala Ile Leu Val Arg Tyr Thr Gln Lys Ala
        1325 1330 1335
Pro Gln Val Ser Thr Pro Thr Leu Val Glu Ala Ala Arg Asn Leu
        1340 1345 1350
Gly Arg Val Gly Thr Lys Cys Cys Thr Leu Pro Glu Asp Gln Arg
        1355 1360 1365
Leu Pro Cys Val Glu Asp Tyr Leu Ser Ala Ile Leu Asn Arg Val
        1370 1375 1380
Cys Leu Leu His Glu Lys Thr Pro Val Ser Glu His Val Thr Lys
        1385 1390 1395
Cys Cys Ser Gly Ser Leu Val Glu Arg Arg Pro Cys Phe Ser Ala
        1400 1405 1410
Leu Thr Val Asp Glu Thr Tyr Val Pro Lys Glu Phe Lys Ala Glu
        1415 1420 1425
Thr Phe Thr Phe His Ser Asp Ile Cys Thr Leu Pro Glu Lys Glu
        1430 1435 1440
Lys Gln Ile Lys Lys Gln Thr Ala Leu Ala Glu Leu Val Lys His
        1445 1450 1455
Lys Pro Lys Ala Thr Ala Glu Gln Leu Lys Thr Val Met Asp Asp
        1460 1465 1470
Phe Ala Gln Phe Leu Asp Thr Cys Cys Lys Ala Ala Asp Lys Asp
        1475 1480 1485
Thr Cys Phe Ser Thr Glu Gly Pro Asn Leu Val Thr Arg Cys Lys
        1490 1495 1500
Asp Ala Leu Ala
        1505
Singly underlined: signal peptide sequence; double-underlined:
beginning and end of NPP3; ** = cleavage position at the signal
peptide sequence; bold residues indicate albumin sequence
ENPP71 Amino Acid Sequence
SEQ. ID NO: 26
Met Arg Gly Pro Ala Val Leu Leu Thr Val Ala Leu Ala Thr Leu Leu
1 5 10 15
Ala Pro Gly Ala Gly Ala**Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                      100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
                        180 185 190
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
                675 680 685
Ile Val Pro Met Tyr Gln Ser Phe Gln Val Ile Trp Arg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp Thr Leu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp
        50
Singly underlined: signal peptide sequence; double-underlined:
beginning and end of NPP1; ** = cleavage position at the signal
peptide sequence
ENPP121 Amino Acid Sequence
SEQ. ID NO: 27
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu 50 Ala Pro Met Asp Val Gly Glu Glu Pro Leu Glu Lys Ala Ala
55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                      100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp
                915 920 925
Singly underlined: signal peptide sequence; double-underlined:
beginning and end of NPP1;
** = cleavage position at the signal peptide sequence
ENPP121-Fc Amino Acid Sequence
SEQ. ID. NO: 28
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                      100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp Leu Ile Asn
                915 920 925
Asp Lys Thr His Thr Cys Pro Pro Cys Pro Ala Pro Glu Leu Leu Gly
        930 935 940
Gly Pro Ser Val Phe Leu Phe Pro Pro Lys Pro Lys Asp Thr Leu Met
945 950 955 960
Ile Ser Arg Thr Pro Glu Val Thr Cys Val Val Val Asp Val Ser His
                                965 970 975
Glu Asp Pro Glu Val Lys Phe Asn Trp Tyr Val Asp Gly Val Glu Val
                        980 985 990
His Asn Ala Lys Thr Lys Pro Arg Glu Glu Gln Tyr Asn Ser Thr Tyr
                995 1000 1005
Arg Val Val Ser Val Leu Thr Val Leu His Gln Asp Trp Leu Asn
        1010 1015 1020
Gly Lys Glu Tyr Lys Cys Lys Val Ser Asn Lys Ala Leu Pro Ala
        1025 1030 1035
Pro Ile Glu Lys Thr Ile Ser Lys Ala Lys Gly Gln Pro Arg Glu
        1040 1045 1050
Pro Gln Val Tyr Thr Leu Pro Pro Ser Arg Glu Glu Met Thr Lys
        1055 1060 1065
Asn Gln Val Ser Leu Thr Cys Leu Val Lys Gly Phe Tyr Pro Ser
        1070 1075 1080
Asp Ile Ala Val Glu Trp Glu Ser Asn Gly Gln Pro Glu Asn Asn
        1085 1090 1095
Tyr Lys Thr Thr Pro Pro Val Leu Asp Ser Asp Gly Ser Phe Phe
        1100 1105 1110
Leu Tyr Ser Lys Leu Thr Val Asp Lys Ser Arg Trp Gln Gln Gly
        1115 1120 1125
Asn Val Phe Ser Cys Ser Val Met His Glu Ala Leu His Asn His
        1130 1135 1140
Tyr Thr Gln Lys Ser Leu Ser Leu Ser Pro Gly Lys
        1145 1150 1155
Singly underlined: signal peptide sequence; double-underlined:
beginning and end of NPP1; ** = cleavage position at the signal
peptide sequence; bold residues indicate Fc sequence
ENPP121-ALB Amino Acid Sequence:
SEQ. ID NO: 29
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                      100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp Arg Ser Gly
                915 920 925
Ser Gly Gly Ser Met Lys Trp Val Thr Phe Leu Leu Leu Leu Phe Val
        930 935 940
Ser Gly Ser Ala Phe Ser Arg Gly Val Phe Arg Arg Glu Ala His Lys
945 950 955 960
Ser Glu Ile Ala His Arg Tyr Asn Asp Leu Gly Glu Gln His Phe Lys
                                965 970 975
Gly Leu Val Leu Ile Ala Phe Ser Gln Tyr Leu Gln Lys Cys Ser Tyr
                        980 985 990
Asp Glu His Ala Lys Leu Val Gln Glu Val Thr Asp Phe Ala Lys Thr
                995 1000 1005
Cys Val Ala Asp Glu Ser Ala Ala Asn Cys Asp Lys Ser Leu His
        1010 1015 1020
Thr Leu Phe Gly Asp Lys Leu Cys Ala Ile Pro Asn Leu Arg Glu
        1025 1030 1035
Asn Tyr Gly Glu Leu Ala Asp Cys Cys Thr Lys Gln Glu Pro Glu
        1040 1045 1050
Arg Asn Glu Cys Phe Leu Gln His Lys Asp Asp Asn Pro Ser Leu
        1055 1060 1065
Pro Pro Phe Glu Arg Pro Glu Ala Glu Ala Met Cys Thr Ser Phe
        1070 1075 1080
Lys Glu Asn Pro Thr Thr Phe Met Gly His Tyr Leu His Glu Val
        1085 1090 1095
Ala Arg Arg His Pro Tyr Phe Tyr Ala Pro Glu Leu Leu Tyr Tyr
        1100 1105 1110
Ala Glu Gln Tyr Asn Glu Ile Leu Thr Gln Cys Cys Ala Glu Ala
        1115 1120 1125
Asp Lys Glu Ser Cys Leu Thr Pro Lys Leu Asp Gly Val Lys Glu
        1130 1135 1140
Lys Ala Leu Val Ser Ser Val Arg Gln Arg Met Lys Cys Ser Ser
        1145 1150 1155
Met Gln Lys Phe Gly Glu Arg Ala Phe Lys Ala Trp Ala Val Ala
        1160 1165 1170
Arg Leu Ser Gln Thr Phe Pro Asn Ala Asp Phe Ala Glu Ile Thr
        1175 1180 1185
Lys Leu Ala Thr Asp Leu Thr Lys Val Asn Lys Glu Cys Cys His
        1190 1195 1200
Gly Asp Leu Leu Glu Cys Ala Asp Asp Arg Ala Glu Leu Ala Lys
        1205 1210 1215
Tyr Met Cys Glu Asn Gln Ala Thr Ile Ser Ser Lys Leu Gln Thr
        1220 1225 1230
Cys Cys Asp Lys Pro Leu Leu Lys Lys Ala His Cys Leu Ser Glu
        1235 1240 1245
Val Glu His Asp Thr Met Pro Ala Asp Leu Pro Ala Ile Ala Ala
        1250 1255 1260
Asp Phe Val Glu Asp Gln Glu Val Cys Lys Asn Tyr Ala Glu Ala
        1265 1270 1275
Lys Asp Val Phe Leu Gly Thr Phe Leu Tyr Glu Tyr Ser Arg Arg
        1280 1285 1290
His Pro Asp Tyr Ser Val Ser Leu Leu Leu Arg Leu Ala Lys Lys
        1295 1300 1305
Tyr Glu Ala Thr Leu Glu Lys Cys Cys Ala Glu Ala Asn Pro Pro
        1310 1315 1320
Ala Cys Tyr Gly Thr Val Leu Ala Glu Phe Gln Pro Leu Val Glu
        1325 1330 1335
Glu Pro Lys Asn Leu Val Lys Thr Asn Cys Asp Leu Tyr Glu Lys
        1340 1345 1350
Leu Gly Glu Tyr Gly Phe Gln Asn Ala Ile Leu Val Arg Tyr Thr
        1355 1360 1365
Gln Lys Ala Pro Gln Val Ser Thr Pro Thr Leu Val Glu Ala Ala
        1370 1375 1380
Arg Asn Leu Gly Arg Val Gly Thr Lys Cys Cys Thr Leu Pro Glu
        1385 1390 1395
Asp Gln Arg Leu Pro Cys Val Glu Asp Tyr Leu Ser Ala Ile Leu
        1400 1405 1410
Asn Arg Val Cys Leu Leu His Glu Lys Thr Pro Val Ser Glu His
        1415 1420 1425
Val Thr Lys Cys Cys Ser Gly Ser Leu Val Glu Arg Arg Pro Cys
        1430 1435 1440
Phe Ser Ala Leu Thr Val Asp Glu Thr Tyr Val Pro Lys Glu Phe
        1445 1450 1455
Lys Ala Glu Thr Phe Thr Phe His Ser Asp Ile Cys Thr Leu Pro
        1460 1465 1470
Glu Lys Glu Lys Gln Ile Lys Lys Gln Thr Ala Leu Ala Glu Leu
        1475 1480 1485
Val Lys His Lys Pro Lys Ala Thr Ala Glu Gln Leu Lys Thr Val
        1490 1495 1500
Met Asp Asp Phe Ala Gln Phe Leu Asp Thr Cys Cys Lys Ala Ala
        1505 1510 1515
Asp Lys Asp Thr Cys Phe Ser Thr Glu Gly Pro Asn Leu Val Thr
        1520 1525 1530
Arg Cys Lys Asp Ala Leu Ala Arg Ser Trp Ser His Pro Gln Phe
        1535 1540 1545
Glu Lys
        1550
Singly underlined: signal peptide sequence; double-underlined:
beginning and end of NPP1; ** = cleavage position at the signal
peptide sequence; bold residues indicate albumin sequence
ENPP121-NPP3-Fc sequence
SEQ. ID NO: 30
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala**Lys
                                85 90 95
Gln Gly Ser Cys Arg Lys Lys Cys Phe Asp Ala Ser Phe Arg Gly Leu
                      100 105 110
Glu Asn Cys Arg Cys Asp Val Ala Cys Lys Asp Arg Gly Asp Cys Cys
                115 120 125
Trp Asp Phe Glu Asp Thr Cys Val Glu Ser Thr Arg Ile Trp Met Cys
        130 135 140
Asn Lys Phe Arg Cys Gly Glu Arg Leu Glu Ala Ser Leu Cys Ser Cys
145 150 155 160
Ser Asp Asp Cys Leu Gln Arg Lys Asp Cys Cys Ala Asp Tyr Lys Ser
                                165 170 175
Val Cys Gln Gly Glu Thr Ser Trp Leu Glu Glu Asn Cys Asp Thr Ala
                        180 185 190
Gln Gln Ser Gln Cys Pro Glu Gly Phe Asp Leu Pro Pro Val Ile Leu
                195 200 205
Phe Ser Met Asp Gly Phe Arg Ala Glu Tyr Leu Tyr Thr Trp Asp Thr
        210 215 220
Leu Met Pro Asn Ile Asn Lys Leu Lys Thr Cys Gly Ile His Ser Lys
225 230 235 240
Tyr Met Arg Ala Met Tyr Pro Thr Lys Thr Phe Pro Asn His Tyr Thr
                                245 250 255
Ile Val Thr Gly Leu Tyr Pro Glu Ser His Gly Ile Ile Asp Asn Asn
                        260 265 270
Met Tyr Asp Val Asn Leu Asn Lys Asn Phe Ser Leu Ser Ser Lys Glu
                275 280 285
Gln Asn Asn Pro Ala Trp Trp His Gly Gln Pro Met Trp Leu Thr Ala
        290 295 300
Met Tyr Gln Gly Leu Lys Ala Ala Thr Tyr Phe Trp Pro Gly Ser Glu
305 310 315 320
Val Ala Ile Asn Gly Ser Phe Pro Ser Ile Tyr Met Pro Tyr Asn Gly
                                325 330 335
Ser Val Pro Phe Glu Glu Arg Ile Ser Thr Leu Leu Lys Trp Leu Asp
                        340 345 350
Leu Pro Lys Ala Glu Arg Pro Arg Phe Tyr Thr Met Tyr Phe Glu Glu
                355 360 365
Pro Asp Ser Ser Gly His Ala Gly Gly Pro Val Ser Ala Arg Val Ile
        370 375 380
Lys Ala Leu Gln Val Val Asp His Ala Phe Gly Met Leu Met Glu Gly
385 390 395 400
Leu Lys Gln Arg Asn Leu His Asn Cys Val Asn Ile Ile Leu Leu Ala
                                405 410 415
Asp His Gly Met Asp Gln Thr Tyr Cys Asn Lys Met Glu Tyr Met Thr
                        420 425 430
Asp Tyr Phe Pro Arg Ile Asn Phe Phe Tyr Met Tyr Glu Gly Pro Ala
                435 440 445
Pro Arg Ile Arg Ala His Asn Ile Pro His Asp Phe Phe Ser Phe Asn
        450 455 460
Ser Glu Glu Ile Val Arg Asn Leu Ser Cys Arg Lys Pro Asp Gln His
465 470 475 480
Phe Lys Pro Tyr Leu Thr Pro Asp Leu Pro Lys Arg Leu His Tyr Ala
                                485 490 495
Lys Asn Val Arg Ile Asp Lys Val His Leu Phe Val Asp Gln Gln Trp
                        500 505 510
Leu Ala Val Arg Ser Lys Ser Asn Thr Asn Cys Gly Gly Gly Asn His
                515 520 525
Gly Tyr Asn Asn Glu Phe Arg Ser Met Glu Ala Ile Phe Leu Ala His
        530 535 540
Gly Pro Ser Phe Lys Glu Lys Thr Glu Val Glu Pro Phe Glu Asn Ile
545 550 555 560
Glu Val Tyr Asn Leu Met Cys Asp Leu Leu Arg Ile Gln Pro Ala Pro
                                565 570 575
Asn Asn Gly Thr His Gly Ser Leu Asn His Leu Leu Lys Val Pro Phe
                        580 585 590
Tyr Glu Pro Ser His Ala Glu Glu Val Ser Lys Phe Ser Val Cys Gly
                595 600 605
Phe Ala Asn Pro Leu Pro Thr Glu Ser Leu Asp Cys Phe Cys Pro His
        610 615 620
Leu Gln Asn Ser Thr Gln Leu Glu Gln Val Asn Gln Met Leu Asn Leu
625 630 635 640
Thr Gln Glu Glu Ile Thr Ala Thr Val Lys Val Asn Leu Pro Phe Gly
                                645 650 655
Arg Pro Arg Val Leu Gln Lys Asn Val Asp His Cys Leu Leu Tyr His
                        660 665 670
Arg Glu Tyr Val Ser Gly Phe Gly Lys Ala Met Arg Met Pro Met Trp
                675 680 685
Ser Ser Tyr Thr Val Pro Gln Leu Gly Asp Thr Ser Pro Leu Pro Pro
        690 695 700
Thr Val Pro Asp Cys Leu Arg Ala Asp Val Arg Val Pro Pro Ser Glu
705 710 715 720
Ser Gln Lys Cys Ser Phe Tyr Leu Ala Asp Lys Asn Ile Thr His Gly
                                725 730 735
Phe Leu Tyr Pro Pro Ala Ser Asn Arg Thr Ser Asp Ser Gln Tyr Asp
                        740 745 750
Ala Leu Ile Thr Ser Asn Leu Val Pro Met Tyr Glu Glu Phe Arg Lys
                755 760 765
Met Trp Asp Tyr Phe His Ser Val Leu Leu Ile Lys His Ala Thr Glu
        770 775 780
Arg Asn Gly Val Asn Val Val Ser Gly Pro Ile Phe Asp Tyr Asn Tyr
785 790 795 800
Asp Gly His Phe Asp Ala Pro Asp Glu Ile Thr Lys His Leu Ala Asn
                                805 810 815
Thr Asp Val Pro Ile Pro Thr His Tyr Phe Val Val Leu Thr Ser Cys
                        820 825 830
Lys Asn Lys Ser His Thr Pro Glu Asn Cys Pro Gly Trp Leu Asp Val
                835 840 845
Leu Pro Phe Ile Ile Pro His Arg Pro Thr Asn Val Glu Ser Cys Pro
        850 855 860
Glu Gly Lys Pro Glu Ala Leu Trp Val Glu Glu Arg Phe Thr Ala His
865 870 875 880
Ile Ala Arg Val Arg Asp Val Glu Leu Leu Thr Gly Leu Asp Phe Tyr
                                885 890 895
Gln Asp Lys Val Gln Pro Val Ser Glu Ile Leu Gln Leu Lys Thr Tyr
                        900 905 910
Leu Pro Thr Phe Glu Thr Thr Ile Asp Lys Thr His Thr Cys Pro Pro
                915 920 925
Cys Pro Ala Pro Glu Leu Leu Gly Gly Pro Ser Val Phe Leu Phe Pro
        930 935 940
Pro Lys Pro Lys Asp Thr Leu Met Ile Ser Arg Thr Pro Glu Val Thr
945 950 955 960
Cys Val Val Val Asp Val Ser His Glu Asp Pro Glu Val Lys Phe Asn
                                965 970 975
Trp Tyr Val Asp Gly Val Glu Val His Asn Ala Lys Thr Lys Pro Arg
                        980 985 990
Glu Glu Gln Tyr Asn Ser Thr Tyr Arg Val Val Ser Val Leu Thr Val
                995 1000 1005
Leu His Gln Asp Trp Leu Asn Gly Lys Glu Tyr Lys Cys Lys Val
        1010 1015 1020
Ser Asn Lys Ala Leu Pro Ala Pro Ile Glu Lys Thr Ile Ser Lys
        1025 1030 1035
Ala Lys Gly Gln Pro Arg Glu Pro Gln Val Tyr Thr Leu Pro Pro
        1040 1045 1050
Ser Arg Glu Glu Met Thr Lys Asn Gln Val Ser Leu Thr Cys Leu
        1055 1060 1065
Val Lys Gly Phe Tyr Pro Ser Asp Ile Ala Val Glu Trp Glu Ser
        1070 1075 1080
Asn Gly Gln Pro Glu Asn Asn Tyr Lys Thr Thr Pro Pro Val Leu
        1085 1090 1095
Asp Ser Asp Gly Ser Phe Phe Leu Tyr Ser Lys Leu Thr Val Asp
        1100 1105 1110
Lys Ser Arg Trp Gln Gln Gly Asn Val Phe Ser Cys Ser Val Met
        1115 1120 1125
His Glu Ala Leu His Asn His Tyr Thr Gln Lys Ser Leu Ser Leu
        1130 1135 1140
Ser Pro Gly Lys
        1145
Singly underlined: signal peptide sequence; double-underlined:
beginning and end of NPP3; ** = cleavage position at the signal
peptide sequence; bold residues indicate Fc sequence
ENPP121-NPP3-Albumin sequence
SEQ. ID NO: 31
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala**Lys
                                85 90 95
Gln Gly Ser Cys Arg Lys Lys Cys Phe Asp Ala Ser Phe Arg Gly Leu
                      100 105 110
Glu Asn Cys Arg Cys Asp Val Ala Cys Lys Asp Arg Gly Asp Cys Cys
                115 120 125
Trp Asp Phe Glu Asp Thr Cys Val Glu Ser Thr Arg Ile Trp Met Cys
        130 135 140
Asn Lys Phe Arg Cys Gly Glu Arg Leu Glu Ala Ser Leu Cys Ser Cys
145 150 155 160
Ser Asp Asp Cys Leu Gln Arg Lys Asp Cys Cys Ala Asp Tyr Lys Ser
                                165 170 175
Val Cys Gln Gly Glu Thr Ser Trp Leu Glu Glu Asn Cys Asp Thr Ala
                        180 185 190
Gln Gln Ser Gln Cys Pro Glu Gly Phe Asp Leu Pro Pro Val Ile Leu
                195 200 205
Phe Ser Met Asp Gly Phe Arg Ala Glu Tyr Leu Tyr Thr Trp Asp Thr
        210 215 220
Leu Met Pro Asn Ile Asn Lys Leu Lys Thr Cys Gly Ile His Ser Lys
225 230 235 240
Tyr Met Arg Ala Met Tyr Pro Thr Lys Thr Phe Pro Asn His Tyr Thr
                                245 250 255
Ile Val Thr Gly Leu Tyr Pro Glu Ser His Gly Ile Ile Asp Asn Asn
                        260 265 270
Met Tyr Asp Val Asn Leu Asn Lys Asn Phe Ser Leu Ser Ser Lys Glu
                275 280 285
Gln Asn Asn Pro Ala Trp Trp His Gly Gln Pro Met Trp Leu Thr Ala
        290 295 300
Met Tyr Gln Gly Leu Lys Ala Ala Thr Tyr Phe Trp Pro Gly Ser Glu
305 310 315 320
Val Ala Ile Asn Gly Ser Phe Pro Ser Ile Tyr Met Pro Tyr Asn Gly
                                325 330 335
Ser Val Pro Phe Glu Glu Arg Ile Ser Thr Leu Leu Lys Trp Leu Asp
                        340 345 350
Leu Pro Lys Ala Glu Arg Pro Arg Phe Tyr Thr Met Tyr Phe Glu Glu
                355 360 365
Pro Asp Ser Ser Gly His Ala Gly Gly Pro Val Ser Ala Arg Val Ile
        370 375 380
Lys Ala Leu Gln Val Val Asp His Ala Phe Gly Met Leu Met Glu Gly
385 390 395 400
Leu Lys Gln Arg Asn Leu His Asn Cys Val Asn Ile Ile Leu Leu Ala
                                405 410 415
Asp His Gly Met Asp Gln Thr Tyr Cys Asn Lys Met Glu Tyr Met Thr
                        420 425 430
Asp Tyr Phe Pro Arg Ile Asn Phe Phe Tyr Met Tyr Glu Gly Pro Ala
                435 440 445
Pro Arg Ile Arg Ala His Asn Ile Pro His Asp Phe Phe Ser Phe Asn
        450 455 460
Ser Glu Glu Ile Val Arg Asn Leu Ser Cys Arg Lys Pro Asp Gln His
465 470 475 480
Phe Lys Pro Tyr Leu Thr Pro Asp Leu Pro Lys Arg Leu His Tyr Ala
                                485 490 495
Lys Asn Val Arg Ile Asp Lys Val His Leu Phe Val Asp Gln Gln Trp
                        500 505 510
Leu Ala Val Arg Ser Lys Ser Asn Thr Asn Cys Gly Gly Gly Asn His
                515 520 525
Gly Tyr Asn Asn Glu Phe Arg Ser Met Glu Ala Ile Phe Leu Ala His
        530 535 540
Gly Pro Ser Phe Lys Glu Lys Thr Glu Val Glu Pro Phe Glu Asn Ile
545 550 555 560
Glu Val Tyr Asn Leu Met Cys Asp Leu Leu Arg Ile Gln Pro Ala Pro
                                565 570 575
Asn Asn Gly Thr His Gly Ser Leu Asn His Leu Leu Lys Val Pro Phe
                        580 585 590
Tyr Glu Pro Ser His Ala Glu Glu Val Ser Lys Phe Ser Val Cys Gly
                595 600 605
Phe Ala Asn Pro Leu Pro Thr Glu Ser Leu Asp Cys Phe Cys Pro His
        610 615 620
Leu Gln Asn Ser Thr Gln Leu Glu Gln Val Asn Gln Met Leu Asn Leu
625 630 635 640
Thr Gln Glu Glu Ile Thr Ala Thr Val Lys Val Asn Leu Pro Phe Gly
                                645 650 655
Arg Pro Arg Val Leu Gln Lys Asn Val Asp His Cys Leu Leu Tyr His
                        660 665 670
Arg Glu Tyr Val Ser Gly Phe Gly Lys Ala Met Arg Met Pro Met Trp
                675 680 685
Ser Ser Tyr Thr Val Pro Gln Leu Gly Asp Thr Ser Pro Leu Pro Pro
        690 695 700
Thr Val Pro Asp Cys Leu Arg Ala Asp Val Arg Val Pro Pro Ser Glu
705 710 715 720
Ser Gln Lys Cys Ser Phe Tyr Leu Ala Asp Lys Asn Ile Thr His Gly
                                725 730 735
Phe Leu Tyr Pro Pro Ala Ser Asn Arg Thr Ser Asp Ser Gln Tyr Asp
                        740 745 750
Ala Leu Ile Thr Ser Asn Leu Val Pro Met Tyr Glu Glu Phe Arg Lys
                755 760 765
Met Trp Asp Tyr Phe His Ser Val Leu Leu Ile Lys His Ala Thr Glu
        770 775 780
Arg Asn Gly Val Asn Val Val Ser Gly Pro Ile Phe Asp Tyr Asn Tyr
785 790 795 800
Asp Gly His Phe Asp Ala Pro Asp Glu Ile Thr Lys His Leu Ala Asn
                                805 810 815
Thr Asp Val Pro Ile Pro Thr His Tyr Phe Val Val Leu Thr Ser Cys
                        820 825 830
Lys Asn Lys Ser His Thr Pro Glu Asn Cys Pro Gly Trp Leu Asp Val
                835 840 845
Leu Pro Phe Ile Ile Pro His Arg Pro Thr Asn Val Glu Ser Cys Pro
        850 855 860
Glu Gly Lys Pro Glu Ala Leu Trp Val Glu Glu Arg Phe Thr Ala His
865 870 875 880
Ile Ala Arg Val Arg Asp Val Glu Leu Leu Thr Gly Leu Asp Phe Tyr
                                885 890 895
Gln Asp Lys Val Gln Pro Val Ser Glu Ile Leu Gln Leu Lys Thr Tyr
                        900 905 910
Leu Pro Thr Phe Glu Thr Thr Ile Gly Gly Gly Ser Gly Gly Gly Gly
                915 920 925
Ser Gly Gly Gly Gly Ser Met Lys Trp Val Thr Phe Leu Leu Leu Leu
        930 935 940
Phe Val Ser Gly Ser Ala Phe Ser Arg Gly Val Phe Arg Arg Glu Ala
945 950 955 960
His Lys Ser Glu Ile Ala His Arg Tyr Asn Asp Leu Gly Glu Gln His
                                965 970 975
Phe Lys Gly Leu Val Leu Ile Ala Phe Ser Gln Tyr Leu Gln Lys Cys
                        980 985 990
Ser Tyr Asp Glu His Ala Lys Leu Val Gln Glu Val Thr Asp Phe Ala
                995 1000 1005
Lys Thr Cys Val Ala Asp Glu Ser Ala Ala Asn Cys Asp Lys Ser
        1010 1015 1020
Leu His Thr Leu Phe Gly Asp Lys Leu Cys Ala Ile Pro Asn Leu
        1025 1030 1035
Arg Glu Asn Tyr Gly Glu Leu Ala Asp Cys Cys Thr Lys Gln Glu
        1040 1045 1050
Pro Glu Arg Asn Glu Cys Phe Leu Gln His Lys Asp Asp Asn Pro
        1055 1060 1065
Ser Leu Pro Pro Phe Glu Arg Pro Glu Ala Glu Ala Met Cys Thr
        1070 1075 1080
Ser Phe Lys Glu Asn Pro Thr Thr Phe Met Gly His Tyr Leu His
        1085 1090 1095
Glu Val Ala Arg Arg His Pro Tyr Phe Tyr Ala Pro Glu Leu Leu
        1100 1105 1110
Tyr Tyr Ala Glu Gln Tyr Asn Glu Ile Leu Thr Gln Cys Cys Ala
        1115 1120 1125
Glu Ala Asp Lys Glu Ser Cys Leu Thr Pro Lys Leu Asp Gly Val
        1130 1135 1140
Lys Glu Lys Ala Leu Val Ser Ser Val Arg Gln Arg Met Lys Cys
        1145 1150 1155
Ser Ser Met Gln Lys Phe Gly Glu Arg Ala Phe Lys Ala Trp Ala
        1160 1165 1170
Val Ala Arg Leu Ser Gln Thr Phe Pro Asn Ala Asp Phe Ala Glu
        1175 1180 1185
Ile Thr Lys Leu Ala Thr Asp Leu Thr Lys Val Asn Lys Glu Cys
        1190 1195 1200
Cys His Gly Asp Leu Leu Glu Cys Ala Asp Asp Arg Ala Glu Leu
        1205 1210 1215
Ala Lys Tyr Met Cys Glu Asn Gln Ala Thr Ile Ser Ser Lys Leu
        1220 1225 1230
Gln Thr Cys Cys Asp Lys Pro Leu Leu Lys Lys Ala His Cys Leu
        1235 1240 1245
Ser Glu Val Glu His Asp Thr Met Pro Ala Asp Leu Pro Ala Ile
        1250 1255 1260
Ala Ala Asp Phe Val Glu Asp Gln Glu Val Cys Lys Asn Tyr Ala
        1265 1270 1275
Glu Ala Lys Asp Val Phe Leu Gly Thr Phe Leu Tyr Glu Tyr Ser
        1280 1285 1290
Arg Arg His Pro Asp Tyr Ser Val Ser Leu Leu Leu Arg Leu Ala
        1295 1300 1305
Lys Lys Tyr Glu Ala Thr Leu Glu Lys Cys Cys Ala Glu Ala Asn
        1310 1315 1320
Pro Pro Ala Cys Tyr Gly Thr Val Leu Ala Glu Phe Gln Pro Leu
        1325 1330 1335
Val Glu Glu Pro Lys Asn Leu Val Lys Thr Asn Cys Asp Leu Tyr
        1340 1345 1350
Glu Lys Leu Gly Glu Tyr Gly Phe Gln Asn Ala Ile Leu Val Arg
        1355 1360 1365
Tyr Thr Gln Lys Ala Pro Gln Val Ser Thr Pro Thr Leu Val Glu
        1370 1375 1380
Ala Ala Arg Asn Leu Gly Arg Val Gly Thr Lys Cys Cys Thr Leu
        1385 1390 1395
Pro Glu Asp Gln Arg Leu Pro Cys Val Glu Asp Tyr Leu Ser Ala
        1400 1405 1410
Ile Leu Asn Arg Val Cys Leu Leu His Glu Lys Thr Pro Val Ser
        1415 1420 1425
Glu His Val Thr Lys Cys Cys Ser Gly Ser Leu Val Glu Arg Arg
        1430 1435 1440
Pro Cys Phe Ser Ala Leu Thr Val Asp Glu Thr Tyr Val Pro Lys
        1445 1450 1455
Glu Phe Lys Ala Glu Thr Phe Thr Phe His Ser Asp Ile Cys Thr
        1460 1465 1470
Leu Pro Glu Lys Glu Lys Gln Ile Lys Lys Gln Thr Ala Leu Ala
        1475 1480 1485
Glu Leu Val Lys His Lys Pro Lys Ala Thr Ala Glu Gln Leu Lys
        1490 1495 1500
Thr Val Met Asp Asp Phe Ala Gln Phe Leu Asp Thr Cys Cys Lys
        1505 1510 1515
Ala Ala Asp Lys Asp Thr Cys Phe Ser Thr Glu Gly Pro Asn Leu
        1520 1525 1530
Val Thr Arg Cys Lys Asp Ala Leu Ala
        1535 1540
Singly underlined: signal peptide sequence; double-underlined:
beginning and end of NPP3; ** = cleavage position at the signal
peptide sequence; bold residues indicate albumin sequence
ENPP121GLK Protein Export Signal Sequence
SEQ. ID NO: 32
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala Gly
                                85 90 95
Leu Lys
Albumin Sequence
SEQ. ID NO: 33
Gly Gly Gly Gly Ser Gly Gly Gly Gly Ser Gly Gly Gly Gly Ser Met
1 5 10 15
Lys Trp Val Thr Phe Leu Leu Leu Leu Phe Val Ser Gly Ser Ala Phe
                        20 25 30
Ser Arg Gly Val Phe Arg Arg Glu Ala His Lys Ser Glu Ile Ala His
                35 40 45
Arg Tyr Asn Asp Leu Gly Glu Gln His Phe Lys Gly Leu Val Leu Ile
        50 55 60
Ala Phe Ser Gln Tyr Leu Gln Lys Cys Ser Tyr Asp Glu His Ala Lys
65 70 75 80
Leu Val Gln Glu Val Thr Asp Phe Ala Lys Thr Cys Val Ala Asp Glu
                                85 90 95
Ser Ala Ala Asn Cys Asp Lys Ser Leu His Thr Leu Phe Gly Asp Lys
                      100 105 110
Leu Cys Ala Ile Pro Asn Leu Arg Glu Asn Tyr Gly Glu Leu Ala Asp
                115 120 125
Cys Cys Thr Lys Gln Glu Pro Glu Arg Asn Glu Cys Phe Leu Gln His
        130 135 140
Lys Asp Asp Asn Pro Ser Leu Pro Pro Phe Glu Arg Pro Glu Ala Glu
145 150 155 160
Ala Met Cys Thr Ser Phe Lys Glu Asn Pro Thr Thr Phe Met Gly His
                                165 170 175
Tyr Leu His Glu Val Ala Arg Arg His Pro Tyr Phe Tyr Ala Pro Glu
                        180 185 190
Leu Leu Tyr Tyr Ala Glu Gln Tyr Asn Glu Ile Leu Thr Gln Cys Cys
                195 200 205
Ala Glu Ala Asp Lys Glu Ser Cys Leu Thr Pro Lys Leu Asp Gly Val
        210 215 220
Lys Glu Lys Ala Leu Val Ser Ser Val Arg Gln Arg Met Lys Cys Ser
225 230 235 240
Ser Met Gln Lys Phe Gly Glu Arg Ala Phe Lys Ala Trp Ala Val Ala
                                245 250 255
Arg Leu Ser Gln Thr Phe Pro Asn Ala Asp Phe Ala Glu Ile Thr Lys
                        260 265 270
Leu Ala Thr Asp Leu Thr Lys Val Asn Lys Glu Cys Cys His Gly Asp
                275 280 285
Leu Leu Glu Cys Ala Asp Asp Arg Ala Glu Leu Ala Lys Tyr Met Cys
        290 295 300
Glu Asn Gln Ala Thr Ile Ser Ser Lys Leu Gln Thr Cys Cys Asp Lys
305 310 315 320
Pro Leu Leu Lys Lys Ala His Cys Leu Ser Glu Val Glu His Asp Thr
                                325 330 335
Met Pro Ala Asp Leu Pro Ala Ile Ala Ala Asp Phe Val Glu Asp Gln
                        340 345 350
Glu Val Cys Lys Asn Tyr Ala Glu Ala Lys Asp Val Phe Leu Gly Thr
                355 360 365
Phe Leu Tyr Glu Tyr Ser Arg Arg His Pro Asp Tyr Ser Val Ser Leu
        370 375 380
Leu Leu Arg Leu Ala Lys Lys Tyr Glu Ala Thr Leu Glu Lys Cys Cys
385 390 395 400
Ala Glu Ala Asn Pro Pro Ala Cys Tyr Gly Thr Val Leu Ala Glu Phe
                                405 410 415
Gln Pro Leu Val Glu Glu Pro Lys Asn Leu Val Lys Thr Asn Cys Asp
                        420 425 430
Leu Tyr Glu Lys Leu Gly Glu Tyr Gly Phe Gln Asn Ala Ile Leu Val
                435 440 445
Arg Tyr Thr Gln Lys Ala Pro Gln Val Ser Thr Pro Thr Leu Val Glu
        450 455 460
Ala Ala Arg Asn Leu Gly Arg Val Gly Thr Lys Cys Cys Thr Leu Pro
465 470 475 480
Glu Asp Gln Arg Leu Pro Cys Val Glu Asp Tyr Leu Ser Ala Ile Leu
                                485 490 495
Asn Arg Val Cys Leu Leu His Glu Lys Thr Pro Val Ser Glu His Val
                        500 505 510
Thr Lys Cys Cys Ser Gly Ser Leu Val Glu Arg Arg Pro Cys Phe Ser
                515 520 525
Ala Leu Thr Val Asp Glu Thr Tyr Val Pro Lys Glu Phe Lys Ala Glu
        530 535 540
Thr Phe Thr Phe His Ser Asp Ile Cys Thr Leu Pro Glu Lys Glu Lys
545 550 555 560
Gln Ile Lys Lys Gln Thr Ala Leu Ala Glu Leu Val Lys His Lys Pro
                                565 570 575
Lys Ala Thr Ala Glu Gln Leu Lys Thr Val Met Asp Asp Phe Ala Gln
                        580 585 590
Phe Leu Asp Thr Cys Cys Lys Ala Ala Asp Lys Asp Thr Cys Phe Ser
                595 600 605
Thr Glu Gly Pro Asn Leu Val Thr Arg Cys Lys Asp Ala Leu Ala
        610 615 620
Human IgG Fc domain, Fc
SEQ. ID NO: 34
Asp Lys Thr His Thr Cys Pro Pro Cys Pro Ala Pro Glu Leu Leu Gly
1 5 10 15
Gly Pro Ser Val Phe Leu Phe Pro Pro Lys Pro Lys Asp Thr Leu Met
                        20 25 30
Ile Ser Arg Thr Pro Glu Val Thr Cys Val Val Val Asp Val Ser His
                35 40 45
Glu Asp Pro Glu Val Lys Phe Asn Trp Tyr Val Asp Gly Val Glu Val
        50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
                                85 90 95
Lys Glu Tyr Lys Cys Lys Val Ser Asn Lys Ala Leu Pro Ala Pro Ile
                      100 105 110
Glu Lys Thr Ile Ser Lys Ala Lys Gly Gln Pro Arg Glu Pro Gln Val
                115 120 125
Tyr Thr Leu Pro Pro Ser Arg Glu Glu Met Thr Lys Asn Gln Val Ser
        130 135 140
Leu Thr Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
                                165 170 175
Val Leu Asp Ser Asp Gly Ser Phe Phe Leu Tyr Ser Lys Leu Thr Val
                        180 185 190
Asp Lys Ser Arg Trp Gln Gln Gly Asn Val Phe Ser Cys Ser Val Met
                195 200 205
His Glu Ala Leu His Asn His Tyr Thr Gln Lys Ser Leu Ser Leu Ser
        210 215 220
Pro Gly Lys
225
Albumin Sequence
SEQ. ID NO: 35
Met Lys Trp Val Thr Phe Leu Leu Leu Leu Phe Val Ser Gly Ser Ala
1 5 10 15
Phe Ser Arg Gly Val Phe Arg Arg Glu Ala His Lys Ser Glu Ile Ala
                        20 25 30
His Arg Tyr Asn Asp Leu Gly Glu Gln His Phe Lys Gly Leu Val Leu
                35 40 45
Ile Ala Phe Ser Gln Tyr Leu Gln Lys Cys Ser Tyr Asp Glu His Ala
        50 55 60
Lys Leu Val Gln Glu Val Thr Asp Phe Ala Lys Thr Cys Val Ala Asp
65 75 80 70
Glu Ser Ala Ala Asn Cys Asp Lys Ser Leu His Thr Leu Phe Gly Asp
                                85 90 95
Lys Leu Cys Ala Ile Pro Asn Leu Arg Glu Asn Tyr Gly Glu Leu Ala
                      100 105 110
Asp Cys Cys Thr Lys Gln Glu Pro Glu Arg Asn Glu Cys Phe Leu Gln
                115 120 125
His Lys Asp Asp Asn Pro Ser Leu Pro Pro Phe Glu Arg Pro Glu Ala
        130 135 140
Glu Ala Met Cys Thr Ser Phe Lys Glu Asn Pro Thr Thr Phe Met Gly
145 150 155 160
His Tyr Leu His Glu Val Ala Arg Arg His Pro Tyr Phe Tyr Ala Pro
                                165 170 175
Glu Leu Leu Tyr Tyr Ala Glu Gln Tyr Asn Glu Ile Leu Thr Gln Cys
                        180 185 190
Cys Ala Glu Ala Asp Lys Glu Ser Cys Leu Thr Pro Lys Leu Asp Gly
                195 200 205
Val Lys Glu Lys Ala Leu Val Ser Ser Val Arg Gln Arg Met Lys Cys
        210 215 220
Ser Ser Met Gln Lys Phe Gly Glu Arg Ala Phe Lys Ala Trp Ala Val
225 230 235 240
Ala Arg Leu Ser Gln Thr Phe Pro Asn Ala Asp Phe Ala Glu Ile Thr
                                245 250 255
Lys Leu Ala Thr Asp Leu Thr Lys Val Asn Lys Glu Cys Cys His Gly
                        260 265 270
Asp Leu Leu Glu Cys Ala Asp Asp Arg Ala Glu Leu Ala Lys Tyr Met
                275 280 285
Cys Glu Asn Gln Ala Thr Ile Ser Ser Lys Leu Gln Thr Cys Cys Asp
        290 295 300
Lys Pro Leu Leu Lys Lys Ala His Cys Leu Ser Glu Val Glu His Asp
305 310 315 320
Thr Met Pro Ala Asp Leu Pro Ala Ile Ala Ala Asp Phe Val Glu Asp
                                325 330 335
Gln Glu Val Cys Lys Asn Tyr Ala Glu Ala Lys Asp Val Phe Leu Gly
                        340 345 350
Thr Phe Leu Tyr Glu Tyr Ser Arg Arg His Pro Asp Tyr Ser Val Ser
                355 360 365
Leu Leu Leu Arg Leu Ala Lys Lys Tyr Glu Ala Thr Leu Glu Lys Cys
        370 375 380
Cys Ala Glu Ala Asn Pro Pro Ala Cys Tyr Gly Thr Val Leu Ala Glu
385 390 395 400
Phe Gln Pro Leu Val Glu Glu Pro Lys Asn Leu Val Lys Thr Asn Cys
                                405 410 415
Asp Leu Tyr Glu Lys Leu Gly Glu Tyr Gly Phe Gln Asn Ala Ile Leu
                        420 425 430
Val Arg Tyr Thr Gln Lys Ala Pro Gln Val Ser Thr Pro Thr Leu Val
                435 440 445
Glu Ala Ala Arg Asn Leu Gly Arg Val Gly Thr Lys Cys Cys Thr Leu
        450 455 460
Pro Glu Asp Gln Arg Leu Pro Cys Val Glu Asp Tyr Leu Ser Ala Ile
465 470 475 480
Leu Asn Arg Val Cys Leu Leu His Glu Lys Thr Pro Val Ser Glu His
                                485 490 495
Val Thr Lys Cys Cys Ser Gly Ser Leu Val Glu Arg Arg Pro Cys Phe
                        500 505 510
Ser Ala Leu Thr Val Asp Glu Thr Tyr Val Pro Lys Glu Phe Lys Ala
                515 520 525
Glu Thr Phe Thr Phe His Ser Asp Ile Cys Thr Leu Pro Glu Lys Glu
        530 535 540
Lys Gln Ile Lys Lys Gln Thr Ala Leu Ala Glu Leu Val Lys His Lys
545 550 555 560
Pro Lys Ala Thr Ala Glu Gln Leu Lys Thr Val Met Asp Asp Phe Ala
                                565 570 575
Gln Phe Leu Asp Thr Cys Cys Lys Ala Ala Asp Lys Asp Thr Cys Phe
                        580 585 590
Ser Thr Glu Gly Pro Asn Leu Val Thr Arg Cys Lys Asp Ala Leu Ala
                595 600 605
Arg Ser Trp Ser His Pro Gln Phe Glu Lys
        610 615
ENPP2 Signal Peptide
SEQ. ID NO: 36
Leu Phe Thr Phe Ala Val Gly Val Asn Ile Cys Leu Gly
1 5 10 15
Phe Thr Ala
SEQ. ID NO: 37
Signal Sequence ENPP7
Met Arg Gly Pro Ala Val Leu Leu Thr Val Ala Leu Ala Thr Leu Leu
1 5 10 15
Ala Pro Gly Ala
                        20
Signal sequence ENPP7
SEQ. ID NO: 38
Met Arg Gly Pro Ala Val Leu Leu Thr Val Ala Leu Ala Thr Leu Leu
1 5 10 15
Ala Pro Gly Ala Gly Ala
                        20
Signal Sequence ENPP1-2-1
SEQ. ID NO: 39
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala
                                85 90 95
exENPP3
SEQ. ID NO: 40
Leu Leu Val Ile Met Ser Leu Gly Leu Gly Leu Gly Leu Gly Leu Arg
1 5 10 15
Lys
Signal Sequence ENPP5:
SEQ. ID NO: 41
Met Thr Ser Lys Phe Leu Leu Val Ser Phe Ile Leu Ala Ala Leu Ser
1 5 10 15
Leu Ser Thr Thr Phe Ser
                        20
Signal Sequence-Azurocidin
SEQ ID NO: 42
Met Thr Arg Leu Thr Val Leu Ala Leu Leu Ala Gly Leu Leu Ala Ser Ser Arg Ala
Linker
SEQ. ID NO: 43
Asp Ser Ser
Linker
SEQ. ID NO: 44
Glu Ser Ser
Linker
SEQ. ID NO: 45
Arg Gln Gln
Linker
SEQ. ID NO: 46
Lys Arg
Linker
SEQ. ID NO: 47
(Arg)ₘ ; m = 0-15
Linker
SEQ. ID NO: 48
Asp Ser Ser Ser Glu Glu Lys Phe Leu Arg Arg Ile Gly Arg Phe Gly
SEQ. ID NO: 49
Linker
Glu Glu Glu Glu Glu Glu Glu Pro Arg Gly Asp Thr
1 5 10
SEQ. ID NO: 50
Linker
Ala Pro Trp His Leu Ser Ser Gln Tyr Ser Arg Thr
1 5 10
Linker
SEQ. ID NO: 51
Ser Thr Leu Pro Ile Pro His Glu Phe Ser Arg Glu
1 5 10
Linker
SEQ. ID NO: 52
Val Thr Lys His Leu Asn Gln Ile Ser Gln Ser Tyr
1 5 10
Linker
SEQ. ID NO: 53
(Glu)ₘ; m = 1-15
Linker
SEQ. ID NO: 54
Leu Ile Asn
Linker
SEQ. ID NO: 55
Gly Gly Ser Gly Gly Ser
1 5
SEQ. ID NO: 56
Linker
Arg Ser Gly Ser Gly Gly Ser
1 5
Linker
SEQ. ID NO: 57
(Asp)ₘ; m = 1-15
1
Linker
SEQ. ID NO: 58
Leu Val Ile Met Ser Leu Gly Leu Gly Leu Gly Leu Gly Leu Arg Lys
1 5 10 15
Linker
SEQ. ID NO: 59
Val Ile Met Ser Leu Gly Leu Gly Leu Gly Leu Gly Leu Arg Lys
1 5 10 15
Linker
SEQ. ID NO: 60
Ile Met Ser Leu Gly Leu Gly Leu Gly Leu Gly Leu Arg Lys
1 5 10
Linker
SEQ. ID NO: 61
Met Ser Leu Gly Leu Gly Leu Gly Leu Gly Leu Arg Lys
1 5 10
Linker
SEQ. ID NO: 62
Ser Leu Gly Leu Gly Leu Gly Leu Gly Leu Arg Lys
1 5 10
Linker
SEQ. ID NO: 63
Leu Gly Leu Gly Leu Gly Leu Gly Leu Arg Lys
1 5 10
Linker
SEQ. ID NO: 64
Gly Leu Gly Leu Gly Leu Gly Leu Arg Lys
1 5 10
Linker
SEQ. ID NO: 65
Leu Gly Leu Gly Leu Gly Leu Arg Lys
1 5
Linker
SEQ. ID NO: 66
Gly Leu Gly Leu Gly Leu Arg Lys
1 5
Linker
SEQ. ID NO: 67
Leu Gly Leu Gly Leu Arg Lys
1 5
Linker
SEQ. ID NO: 68
Gly Leu Gly Leu Arg Lys
1 5
Linker
SEQ. ID NO: 69
Leu Gly Leu Arg Lys
1 5
Linker
SEQ. ID NO: 70
Gly Leu Arg Lys
Linker
SEQ. ID NO: 71
Leu Arg Lys
1
Linker
SEQ. ID NO: 72
Arg Lys
1
Linker
SEQ. ID NO: 73
(Lys)ₘ; m = 0-15
1
Linker
SEQ. ID NO: 74
Dₘ; m = 1-15
Linker
SEQ. ID NO: 75
(GGGGS)ₙ; n = 1-10
ENPP3 Nucleotide sequence
SEQ. ID NO: 76
atggaatcta cgttgacttt agcaacggaa caacctgtta agaagaacac tottaagaaa 60
tataaaatag cttgcattgt tcttcttgct ttgctggtga tcatgtcact tggattaggc 120
ctggggcttg gactcaggaa actggaaaag caaggcagct gcaggaagaa gtgctttgat 180
gcatcattta gaggactgga gaactgccgg tgtgatgtgg catgtaaaga ccgaggtgat 240
tgctgctggg attttgaaga cacctgtgtg gaatcaactc gaatatggat gtgcaataaa 300
tttcgttgtg gagagaccag attagaggcc agcctttgct cttgttcaga tgactgtttg 360
cagaggaaag attgctgtgc tgactataag agtgtttgcc aaggagaaac ctcatggctg 420
gaagaaaact gtgacacagc ccagcagtct cagtgcccag aagggtttga cctgccacca 480
gttatcttgt tttctatgga tggatttaga gctgaatatt tatacacatg ggatacttta 540
atgccaaata tcaataaact gaaaacatgt ggaattcatt caaaatacat gagagctatg 600
tatcctacca aaaccttccc aaatcattac accattgtca cgggcttgta tccagagtca 660
catggcatca ttgacaataa tatgtatgat gtaaatctca acaagaattt ttcactttct 720
tcaaaggaac aaaataatcc agcctggtgg catgggcaac caatgtggct gacagcaatg 780
tatcaaggtt taaaagccgc tacctacttt tggcccggat cagaagtggc tataaatggc 840
tcctttcctt ccatatacat gccttacaac ggaagtgtcc catttgaaga gaggatttct 900
acactgttaa aatggctgga cctgcccaaa gctgaaagac ccaggtttta taccatgtat 960
tttgaagaac ctgattoctc tggacatgca ggtggaccag tcagtgccag agtaattaaa 1020
gccttacagg tagtagatca tgcttttggg atgttgatgg aaggcctgaa gcagcggaat 1080
ttgcacaact gtgtcaatat catccttctg gctgaccatg gaatggacca gacttattgt 1140
aacaagatgg aatacatgac tgattatttt cccagaataa acttcttcta catgtacgaa 1200
gggcctgccc cccgcatccg agctcataat atacctcatg acttttttag ttttaattct 1260
gaggaaattg ttagaaacct cagttgccga aaacctgatc agcatttcaa gccctatttg 1320
actcctgatt tgccaaagcg actgcactat gccaagaacg tcagaatcga caaagttcat 1380
ctctttgtgg atcaacagtg gctggctgtt aggagtaaat caaatacaaa ttgtggagga 1440
ggcaaccatg gttataacaa tgagtttagg agcatggagg ctatctttct ggcacatgga 1500
cccagtttta aagagaagac tgaagttgaa ccatttgaaa atattgaagt ctataaccta 1560
atgtgtgatc ttctacgcat tcaaccagca ccaaacaatg gaacccatgg tagtttaaac 1620
catcttctga aggtgccttt ttatgagcca tcccatgcag aggaggtgtc aaagttttct 1680
gtttgtggct ttgctaatcc attgcccaca gagtctcttg actgtttctg ccctcaccta 1740
caaaatagta ctcagctgga acaagtgaat cagatgctaa atctcaccca agaagaaata 1800
acagcaacag tgaaagtaaa tttgccattt gggaggccta gggtactgca gaagaacgtg 1860
gaccactgtc tcctttacca cagggaatat gtcagtggat ttggaaaagc tatgaggatg 1920
cccatgtgga gttcatacac agtcccccag ttgggagaca catcgcctct gcctcccact 1980
gtcccagact gtctgcgggc tgatgtcagg gttcctcctt ctgagagcca aaaatgttcc 2040
ttctatttag cagacaagaa tatcacccac ggcttcctct atcctcctgc cagcaataga 2100
acatcagata gccaatatga tgctttaatt actagcaatt tggtacctat gtatgaagaa 2160
ttcagaaaaa tgtgggacta cttccacagt gttcttctta taaaacatgc cacagaaaga 2220
aatggagtaa atgtggttag tggaccaata tttgattata attatgatgg ccattttgat 2280
gctccagatg aaattaccaa acatttagcc aacactgatg ttcccatccc aacacactac 2340
tttgtggtgc tgaccagttg taaaaacaag agccacacac cggaaaactg ccctgggtgg 2400
ctggatgtcc taccctttat catccctcac cgacctacca acgtggagag ctgtcctgaa 2460
ggtaaaccag aagctctttg ggttgaagaa agatttacag ctcacattgc ccgggtccgt 2520
gatgtagaac ttctcactgg gcttgacttc tatcaggata aagtgcagcc tgtctctgaa 2580
attttgcaac taaagacata tttaccaaca tttgaaacca ctatt 2625
ENPP1 Nucleotide sequence:
SEQ. ID NO: 77
atggaacggg acggctgtgc cggcggagga tcaagaggcg gagaaggcgg cagagcccct 60
agagaaggac ctgccggcaa cggcagagac agaggcagat ctcatgccgc cgaagcccct 120
ggcgatcctc aggctgctgc ttctctgctg gcccccatgg atgtgggcga ggaacctctg 180
gaaaaggccg ccagagccag aaccgccaag gaccccaaca cctacaaggt gctgagcctg 240
gtgctgtccg tgtgcgtgct gaccaccatc ctgggctgca tcttcggcct gaagcccagc 300
tgcgccaaag aagtgaagtc ctgcaagggc cggtgcttcg agcggacctt cggcaactgc 360
agatgcgacg ccgcctgtgt ggaactgggc aactgctgcc tggactacca ggaaacctgc 420
atcgagcccg agcacatctg gacctgcaac aagttcagat gcggcgagaa gcggctgacc 480
agatccctgt gtgcctgcag cgacgactgc aaggacaagg gcgactgctg catcaactac 540
agcagcgtgt gccagggcga gaagtcctgg gtggaagaac cctgcgagag catcaacgag 600
ccccagtgcc ctgccggctt cgagacacct cctaccctgc tgttcagcct ggacggcttt 660
cgggccgagt acctgcacac atggggaggc ctgctgcccg tgatcagcaa gctgaagaag 720
tgcggcacct acaccaagaa catgcggccc gtgtacccca ccaagacctt ccccaaccac 780
tactccatcg tgaccggcct gtaccccgag agccacggca tcatcgacaa caagatgtac 840
gaccccaaga tgaacgccag cttcagectg aagtccaaag agaagttcaa ccccgagtgg 900
tataagggcg agcccatctg ggtcaccgcc aagtaccagg gcctgaaaag cggcacattc 960
ttttggcccg gcagcgacgt ggaaatcaac ggcatcttcc ccgacatcta taagatgtac 1020
aacggcagcg tgcccttcga ggaacggatc ctggctgtgc tgcagtggct gcagctgccc 1080
aaggatgagc ggccccactt ctacaccctg tacctggaag aacctgacag cagcggccac 1140
agctacggcc ctgtgtccag cgaagtgatc aaggccctgc agcgggtgga cggcatggtg 1200
ggaatgctga tggacggcct gaaagagctg aacctgcaca gatgcctgaa cctgatectg 1260
atcagcgacc acggcatgga acagggatcc tgcaagaagt acatctacct gaacaagtac 1320
ctgggcgacg tgaagaacat caaagtgatc tacggcccag ccgccagact gaggcctagc 1380
gacgtgcccg acaagtacta cagcttcaac tacgagggaa tcgcccggaa cctgagctgc 1440
agagagccca accagcactt caagccctac ctgaagcact tcctgcccaa gcggctgcac 1500
ttogccaaga gcgacagaat cgagcccctg accttctacc tggaccccca gtggcagctg 1560
gccctgaatc ccagcgagag aaagtactgc ggcagcggct tccacggctc cgacaacgtg 1620
ttcagcaaca tgcaggccct gttcgtgggc tacggacccg gctttaagca cggcatcgag 1680
gccgacacct tcgagaacat cgaggtgtac aatctgatgt gcgacctgct gaatctgacc 1740
cctgccccca acaatggcac ccacggcage ctgaaccatc tgctgaagaa ccccgtgtac 1800
acccctaagc accccaaaga ggtgcacccc ctggtgcagt gccccttcac cagaaacccc 1860
agagacaacc tgggctgtag ctgcaacccc agcatcctgc ccatcgagga cttccagacc 1920
cagttcaacc tgaccgtggc cgaggaaaag atcatcaagc acgagacact gccctacggc 1980
agaccccggg tgctgcagaa agagaacacc atctgcctgc tgagccagca ccagttcatg 2040
agcggctact cccaggacat cctgatgccc ctgtggacca gctacaccgt ggaccggaac 2100
gacagcttct ccaccgagga tttcagcaac tgcctgtacc aggatttccg gatccccctg 2160
agccccgtgc acaagtgcag cttctacaag aacaacacca aggtgtccta cggcttcctg 2220
agccctcccc agctgaacaa gaacagctcc ggcatctaca gcgaggccct gctgactacc 2280
aacatcgtgc ccatgtacca gagcttccaa gtgatctggc ggtacttcca cgacaccctg 2340
ctgcggaagt acgccgaaga acggaacggc gtgaacgtgg tgtccggccc agtgttcgac 2400
ttcgactacg acggcagatg tgacagcctg gaaaatctgc ggcagaaaag aagagtgatc 2460
cggaaccagg aaattctgat ccctacccac ttctttatcg tgctgacaag ctgcaaggat 2520
accagccaga cccccctgca ctgcgagaac ctggataccc tggccttcat cctgcctcac 2580
cggaccgaca acagcgagag ctgtgtgcac ggcaagcacg acagctcttg ggtggaagaa 2640
ctgctgatgc tgcaccgggc cagaatcacc gatgtggaac acatcaccgg cctgagcttt 2700
taccagcagc ggaaagaacc cgtgtccgat atcctgaagc tgaaaaccca tctgcccacc 2760
ttcagccagg aagat 2775
Azurocidin-ENPP1-FC Nucleotide sequence
SEQ ID NO: 78
ggtaccgccaccatgacaagactgacagtgctggctctgctggccggactgttggcctcttctagagctg
ctccttcctgcgccaaagaagtgaagtcctgcaagggcagatgcttcgagcggaccttcggcaactgtag
atgtgacgccgcttgcgtggaactgggcaactgctgcctggactaccaagagacatgcatcgagcccgag
cacatctggacctgcaacaagttcagatgcggcgagaagcggctgaccagatctctgtgcgcctgctctg
acgactgcaaggacaagggcgactgctgcatcaactactcctctgtgtgccagggcgagaagtcctgggt
tgaagaaccctgcgagtccatcaacgagcctcagtgtcctgccggcttcgagacacctcctactctgctg
ttctccctggatggcttcagagccgagtacctgcatacttggggaggcctgctgccagtgatctccaagc
tgaagaagtgcggcacctacaccaagaacatgaggcctgtgtaccctaccaagacattccccaaccacta
ctccatcgtgaccggcctgtatcctgagagccacggcatcatcgacaacaagatgtacgaccccaagatg
aacgcctccttcagcctgaagtccaaagagaagttcaaccccgagtggtataagggcgagcctatctggg
tcaccgctaagtaccagggactgaagtctggcaccttcttttggcctggctccgacgtggaaatcaacgg
catcttccccgacatctataagatgtacaacggctccgtgcctttcgaggaacgcattctggctgttctg
cagtggctgcagctgcctaaggatgagaggcctcacttctacaccctgtacctggaagaacctgactcct
ccggccactcttatggccctgtgtcctctgaagtgatcaaggccctgcagcgagtggacggaatggtcgg
aatgctgatggacggcctgaaagagctgaacctgcacagatgcctgaacctgatcctgatctccgaccac
ggcatggaacaggggagctgcaagaagtacatctacctgaacaagtacctgggcgacgtgaagaacatca
aagtgatctacggcccagccgccagactgaggccttctgatgtgcctgacaagtactactccttcaacta
cgagggaatcgcccggaacctgtcctgcagagagcctaaccagcacttcaagccctacctgaagcacttt
ctgcctaagcggctgcacttcgccaagtctgacagaatcgagcccctgaccttctatctggaccctcagt
ggcagctggccctgaatcctagcgagagaaagtactgtggctccggcttccacggctccgacaacgtgtt
ctctaatatgcaggccctgttcgtcggctacggccctggctttaaacacggcatcgaggccgacaccttc
gagaacatcgaggtgtacaatctgatgtgtgacctgctgaatctgacccctgctcctaacaacggcaccc
acggatctctgaaccatctgctgaagaatcccgtgtacacccctaagcaccccaaagaggttcaccctct
ggtccagtgtcctttcaccagaaatcctcgggacaacctgggctgctcttgcaacccttctatcctgcct
atcgaggactttcagacccagttcaacctgaccgtggccgaggaaaagatcatcaagcacgagacactgc
cctacggcagacctagagtgctgcagaaagagaacaccatctgcctgctgtcccagcaccagttcatgtc
cggctactcccaggacatcctgatgcctctgtggacctcctacaccgtggaccggaacgatagcttctcc
accgaggacttcagcaactgcctgtaccaggatttcagaatccctctgagccccgtgcacaagtgcagct
tctacaagaacaacaccaaggtgtcctacggcttcctgtctcctccacagctgaacaagaactccagcgg
catctactctgaggccctgctgaccaccaacatcgtgcccatgtaccagtccttccaagtgatctggcgg
tacttccacgacaccctgctgaggaagtacgccgaagaaagaaacggcgtgaacgtggtgtctggccccg
tgttcgacttcgactacgacggcagatgcgactctctggaaaacctgcggcagaaaagacgagtgatccg
gaatcaagagatcctgattcctacacacttctttatcgtgctgaccagctgcaaggatacctctcagacc
cctctgcactgcgagaatctggacaccctggccttcattctgcctcacagaaccgacaactccgagtcct
gtgtgcacggcaagcacgactcctcttgggtcgaagaactgctgatgctgcaccgggccagaatcaccga
tgtggaacacatcaccggcctgagcttctaccagcagcggaaagaacctgtgtccgatatcctgaagctg
aaaacccatctgccaaccttcagccaagaggacctgatcaacgacaagacccacacctgtcctccatgtc
ctgctccagaactgctcggaggcccctctgtgttcctgtttccacctaagccaaaggacacactgatgat
ctctcggacccctgaagtgacctgcgtggtggtggatgtgtctcacgaagatcccgaagtcaagttcaat
tggtacgtggacggcgtggaagtgcacaacgccaagaccaagcctagagaggaacagtacaactccacct
acagagtggtgtccgtgctgactgtgctgcaccaggattggctgaacggcaaagagtacaagtgcaaagt
gtccaacaaggctctgcccgctcctatcgaaaagaccatctccaaggctaagggccagcctcgggaacct
caggtttacaccctgcctccatctcgggaagagatgaccaagaaccaggtgtccctgacctgcctggtca
agggcttctacccttccgatatcgccgtggaatgggagtccaatggccagcctgagaacaactacaagac
aacccctcctgtgctggacagcgacggctcattcttcctgtactctaagctgacagtggacaagtcccgg
tggcagcaaggcaatgtgttttcctgctctgtgatgcacgaggccctccacaatcactacacccagaagt
ccctgtctctgtcccctggcaaatgatagctcgag
Legend-
bold = start/stop codon; underlined = nucleotide
sequence of signal peptide.
Azurocidin-ENPP3-FC Nucleotide sequence
SEQ ID NO: 79
atgaccagactgaccgtgctggccctgctggccggcctgctggccagcagcagagccgccaagca
gggcagctgcagaaagaagtgcttcgacgccagcttcagaggcctggagaactgcagatgcgacgtggcc
tgcaaggacagaggcgactgctgctgggacttcgaggacacctgcgtggagagcaccagaatctggatgt
gcaacaagttcagatgcggcgagaccagactggaggccagcctgtgcagctgcagcgacgactgcctgca
gagaaaggactgctgcgccgactacaagagcgtgtgccagggcgagaccagctggctggaggagaactgc
gacaccgcccagcagagccagtgccccgagggcttcgacctgccccccgtgatcctgttcagcatggacg
gcttcagagccgagtacctgtacacctgggacaccctgatgcccaacatcaacaagctgaagacctgcgg
catccacagcaagtacatgagagccatgtaccccaccaagaccttccccaaccactacaccatcgtgacc
ggcctgtaccccgagagccacggcatcatcgacaacaacatgtacgacgtgaacctgaacaagaacttca
gcctgagcagcaaggagcagaacaaccccgcctggtggcacggccagcccatgaacctgaccgccatgta
ccagggcctgaaggccgccacctacttctggcccggcagcgaggtggccatcaacggcagcttccccagc
atctacatgccctacaacggcagcgtgcccttcgaggagagaatcagcaccctgctgaagtggctggacc
tgcccaaggccgagagacccagattctacaccatgtacttcgaggagcccgacagcagcggccacgccgg
cggccccgtgagcgccagagtgatcaaggccctgcaggtggtggaccacgccttcggcatgctgatggag
ggcctgaagcagagaaacctgcacaactgcgtgaacatcatcctgctggccgaccacggcatggaccaga
cctactgcaacaagatggagtacatgaccgactacttccccagaatcaacttcttctacatgtacgaggg
ccccgcccccagaatcagagcccacaacatcccccacgacttcttcagcttcaacagcgaggagatcgtg
agaaacctgagctgcagaaagcccgaccagcacttcaagccctacctgacccccgacctgcccaagagac
tgcactacgccaagaacgtgagaatcgacaaggtgcacctgttcgtggaccagcagtggctggccgtgag
aagcaagagcaacaccaactgcggcggcggcaaccacggctacaacaacgagttcagaagcatggaggcc
atcttcctggcccacggccccagcttcaaggagaagaccgaggtggagcccttcgagaacatcgaggtgt
acaacctgatgtgcgacctgctgagaatccagcccgcccccaacaacggcacccacggcagcctgaacca
cctgctgaaggtgcccttctacgagcccagccacgccgaggaggtgagcaagttcagcgtgtgcggcttc
gccaaccccctgcccaccgagagcctggactgcttctgcccccacctgcagaacagcacccagctggagc
aggtgaaccagatgctgaacctgacccaggaggagatcaccgccaccgtgaaggtgaacctgcccttcgg
cagacccagagtgctgcagaagaacgtggaccactgcctgctgtaccacagagagtacgtgagcggcttc
ggcaaggccatgagaatgcccatgtggagcagctacaccgtgccccagctgggcgacaccagccccctgc
cccccaccgtgcccgactgcctgagagccgacgtgagagtgccccccagcgagagccagaagtgcagctt
ctacctggccgacaagaacatcacccacggcttcctgtacccccccgccagcaacagaaccagcgacagc
cagtacgacgccctgatcaccagcaacctggtgcccatgtacgaggagttcagaaagatgtgggactact
tccacagcgtgctgctgatcaagcacgccaccgagagaaacggcgtgaacgtggtgagcggccccatctt
cgactacaactacgacggccacttcgacgcccccgacgagatcaccaagcacctggccaacaccgacgtg
cccatccccacccactacttcgtggtgctgaccagctgcaagaacaagagccacacccccgagaactgcc
ccggctggctggacgtgctgcccttcatcatcccccacagacccaccaacgtggagagctgccccgaggg
caagcccgaggccctgtgggtggaggagagattcaccgcccacatcgccagagtgagagacgtggagctg
ctgaccggcctggacttctaccaggacaaggtgcagcccgtgagcgagatcctgcagctgaagacctacc
tgcccaccttcgagaccaccatcgacaagacccacacctgccccccctgccccgcccccgagctgctggg
cggccccagcgtgttcctgttcccccccaagcccaaggacaccctgatgatcagcagaacccccgaggtg
acctgcgtggtggtggacgtgagccacgaggaccccgaggtgaagttcaactggtacgtggacggcgtgg
aggtgcacaacgccaagaccaagcccagagaggagcagtacaacagcacctacagagtggtgagcgtgct
gaccgtgctgcaccaggactggctgaacggcaaggagtacaagtgcaaggtgagcaacaaggccctgccc
gcccccatcgagaagaccatcagcaaggccaagggccagcccagagagccccaggtgtacaccctgcccc
ccagcagagaggagatgaccaagaaccaggtgagcctgacctgcctggtgaagggcttctaccccagcga
catcgccgtggagtgggagagcaacggccagcccgagaacaactacaagaccaccccccccgtgctggac
agcgacggcagcttcttcctgtacagcaagctgaccgtggacaagagcagatggcagcagggcaacgtgt
tcagctgcagcgtgatgcacgaggccctgcacaaccactacacccagaagagcctgagcctgagccccgg
caag
```

### Cloning and Expression of ENPP1 and ENPP3 Fusion Polypeptides

ENPP1, or an ENPP1 polypeptide, is prepared as described in US 2015/0359858 A1, which is incorporated herein in its entirety by reference. ENPP1 is a transmembrane protein localized to the cell surface with distinct intramembrane domains. In order to express ENPP1 as a soluble extracellular protein, the transmembrane domain of ENPP1 may be swapped for the transmembrane domain of ENPP2 or a signal peptide sequence such as Azurocidin, which results in the accumulation of soluble, recombinant ENPP1 in the extracellular fluid of the baculovirus cultures. Signal sequences of any other known proteins may be used to target the extracellular domain of ENPP1 for secretion as well, such as but not limited to the signal sequence of the immunoglobulin kappa and lambda light chain proteins. Further, the disclosure should not be construed to be limited to the polypeptides described herein, but also includes polypeptides comprising any enzymatically active truncation of the ENPP1 extracellular domain.

ENPP1 is made soluble by omitting the transmembrane domain. Human ENPP1 (SEQ ID NO:1) was modified to express a soluble, recombinant protein by replacing its transmembrane region (e.g., residues 77-98) with the corresponding subdomain of human ENPP2 (NCBI accession NP 00112433 5, e.g., residues 12-30) or Azurocidin signal sequence (SEQ ID 42).

The modified ENPP1 sequence was cloned into a modified pFastbac FIT vector possessing a TEV protease cleavage site followed by a C-terminus 9-F lIS tag, and cloned and expressed in insect cells, and both proteins were expressed in a baculovirus system as described previously (Albright, et al., 2012, Blood 120:4432-4440; Saunders, et al., 2011, J. Biol. Chem. 18:994-1004; Saunders, et al., 2008, Mol. Cancer Ther. 7:3352-3362), resulting in the accumulation of soluble, recombinant protein in the extracellular fluid.

ENPP3 is poorly exported to the cell surface. Soluble ENPP3 polypeptide is constructed by replacing the signal sequence of ENPP3 with the native signal sequence of other ENPPs or Azurocidin or suitable signal sequences. Several examples of ENPP3 fusion constructs are disclosed in WO 2017/087936. Soluble ENPP3 constructs are prepared by using the signal export signal sequence of other ENPP enzymes, such as but not limited to ENPP7 and/or ENPPS. Soluble ENPP3 constructs are prepared using a signal sequence comprised of a combination of the signal sequences of ENPP1 and ENPP2 (“ENPP1-2-1” or “ENPP121” hereinafter). Signal sequences of any other known proteins may be used to target the extracellular domain of ENPP3 for secretion as well, such as but not limited to the signal sequence of the immunoglobulin kappa and lambda light chain proteins. Further, the disclosure should not be construed to be limited to the constructs described herein, but also includes constructs comprising any enzymatically active truncation of the ENPP3 extracellular domain.

In certain embodiments, the ENPP3 polypeptide is soluble. In some embodiments, the polypeptide of the disclosure includes an ENPP3 polypeptide that lacks the ENPP3 transmembrane domain. In another embodiment, the polypeptide of the disclosure includes an ENPP3 polypeptide wherein the ENPP3 transmembrane domain has been removed and replaced with the transmembrane domain of another polypeptide, such as, by way of non-limiting example, ENPP2, ENPPS or ENPP7 or Azurocidin signal sequence.

In some embodiments, the polypeptide of the disclosure comprises an IgG Fc domain. In certain embodiments, the polypeptide of the disclosure comprises an albumin domain. In other embodiments, the albumin domain is located at the C terminal region of the ENPP3 polypeptide. In yet other embodiments, the IgG Fc domain is located at the C terminal region of the ENPP3 polypeptide. In yet other embodiments, the presence of IgG Fc domain or albumin domain improves half-life, solubility, reduces immunogenicity and increases the activity of the ENPP3 polypeptide.

In certain embodiments, the polypeptide of the disclosure comprises a signal peptide resulting in the secretion of a precursor of the ENPP3 polypeptide, which undergoes proteolytic processing to yield the ENPP3 polypeptide. In other embodiments, the signal peptide is selected from the group consisting of signal peptides of ENPP2, ENPP5 and ENPP7. In yet other embodiments, the signal peptide is selected from the group consisting of SEQ ID NOs: 36-42.

In certain embodiments, the IgG Fc domain or the albumin domain is connected to the C terminal region of the ENPP3 polypeptide by a linker region. In other embodiments, the linker is selected from SEQ ID NOs: 43-75, where n is an integer ranging from 1-20.

### Production and Purification of ENPP1 and ENPP3 Fusion Polypeptides

To produce soluble, recombinant ENPP1 polypeptide for in vitro use, polynucleotide encoding the extracellular domain of ENPP1 (Human NPP1 (NCBI accession NP 006199)) was fused to the Fc domain of IgG (referred to as “ENPP1-Fc”) and was expressed in stable CHO cell lines. In some embodiments, ENPP1 polynucleotide encoding residues 96 to 925 of NCBI accession NP 006199 were fused to Fc domain to generate ENPP1 polypeptide.

Alternately the ENPP1 polypeptide can also be expressed from HEK293 cells, Baculovirus insect cell system or CHO cells or Yeast Pichia expression system using suitable vectors. The ENPP1 polypeptide can be produced in either adherent or suspension cells. Preferably the ENPP1 polypeptide is expressed in CHO cells. To establish stable cell lines the nucleic acid sequence encoding ENPP1 constructs are cloned into an appropriate vector for large scale protein production.

ENPP3 is produced by establishing stable transfections in either CHO or HEK293 mammalian cells. ENPP3 polynucleotide encoding ENPP3 (Human NPP3 (UniProtKB/Swiss-Prot: 014638.2) was fused to the Fc domain of IgG (referred to as “ENPP3-Fc”) and was expressed in stable CHO cell lines. In some embodiments, ENPP3 polynucleotide encoding residues 49-875 of UniProtKB/Swiss-Prot: 014638.2 was fused to Fc domain to generate ENPP3 polypeptide. The ENPP3 polypeptide can be produced in either adherent or suspension cells. To establish stable cell lines the nucleic acid sequence encoding NPP3 fusion polypeptides of the disclosure into an appropriate vector for large scale protein production. There are a variety of these vectors available from commercial sources and any of those can be used. ENPP3 polypeptides are produced following the protocols established in WO 2017/087936, the contents of which are hereby incorporated by reference in their entirety. ENPP1 polypeptides are produced following the protocols established in Albright, et al, 2015, Nat Commun. 6:10006, the contents of which are hereby incorporated by reference in their entirety.

A suitable plasmid containing the desired polypeptide constructs of ENPP1 or ENPP3 can be stably transfected into expression plasmid using established techniques such as electroporation or lipofectamine, and the cells can be grown under antibiotic selection to enhance for stably transfected cells. Clones of single, stably transfected cells are then established and screened for high expressing clones of the desired fusion protein. Screening of the single cell clones for ENPP1 or ENPP3 polypeptide expression can be accomplished in a high-throughput manner in 96 well plates using the synthetic enzymatic substrate pNP-TMP as previously described (Saunders, et al, 2008, Mol. Cancer Therap. 7(10):3352-62; Albright, et al, 2015, Nat Commun. 6:10006).

Upon identification of high expressing clones for ENPP3 or ENPP1 polypeptides through screening, protein production can be accomplished in shaking flasks or bio-reactors previously described for ENPP1 (Albright, et al, 2015, Nat Commun. 6:10006). Purification of ENPP3 or ENPP1 polypeptides can be accomplished using a combination of standard purification techniques known in the art. These techniques are well known in art and are selected from techniques such as column chromatograph, ultracentrifugation, filtration, and precipitation. Column chromatographic purification is accomplished using affinity chromatography such as protein-A and protein-G resins, metal affinity resins such as nickel or copper, hydrophobic exchange chromatography, and reverse-phase high-pressure chromatography (HPLC) using C8-C14 resins. Ion exchange may also be employed, such as anion and cation exchange chromatography using commercially available resins such as Q-sepharose (anion exchange) and SP-sepharose (cation exchange), blue sepharose resin and blue-sephadex resin, and hydroxyapatite resins. Size exclusion chromatography using commercially available S-75 and S200 Superdex resins can also be employed, as known in the art. Buffers used to solubilize the protein and provide the selection media for the above described chromatographic steps, are standard biological buffers known to practitioners of the art and science of protein chemistry.

Some examples of buffers that are used in preparation include citrate, phosphate, acetate, tris(hydroxymemyl)aminomethane, saline buffers, glycine-HCL buffers, Cacodylate buffers, and sodium barbital buffers, which are well known in art. Using a single techniques, or a series of techniques in combination, and the appropriate buffer systems purified ENPP3 and the crude starting material side by side on a Coomasie stained polyacrylamide gel after a single purification step. The ENPP3 protein can then be additionally purified using additional techniques and/or chromatographic steps as described above, to reach substantially higher purity such as ˜99% purity adjusted to the appropriate pH, one can purify the ENPP1 or ENPP3 polypeptides described to greater than 99% purity from crude material.

Following purification, ENPP1-Fc or ENPP3-Fc was dialyzed into PBS supplemented with Zn2+ and Mg2+(PBSplus) concentrated to between 5 and 7 mg/ml, and frozen at −80° C. in aliquots of 200-500 μl. Aliquots were thawed immediately prior to use and the specific activity of the solution was adjusted to 31.25 au/ml (or about 0.7 mg/ml depending on the preparation) by dilution in PBSplus.

### Dosage & Mode of Administration

In another embodiment, the hsNPP1 or hsNPP3 is administered in one or more doses containing about 1.0 mg/kg to about 5.0 mg/kg NPP1 or about 1.0 mg/kg to about 5.0 mg/kg NPP3 respectively. In another embodiment, the hsNPP1 or hsNPP3 is administered in one or more doses containing about 1.0 mg/kg to about 10.0 mg/kg NPP1 or about 1.0 mg/kg to about 10.0 mg/kg NPP3.

The time period between doses of the hsNPP1 or hsNPP3 is at least 2 days and can be longer, for example at least 3 days, at least 1 week, 2 weeks or 1 month. In one embodiment, the administration is weekly, bi-weekly, or monthly.

The recombinant hsNPP1 or hsNPP3 can be administered in any suitable way, such as intravenously, subcutaneously, or intraperitoneally.

The recombinant hsNPP1 or hsNPP3 can be administered in combination with one or more additional therapeutic agents. Exemplary therapeutic agents include, but are not limited to Bisphosphonate, Statins, Fibrates, Niacin, Aspirin, Clopidogrel, and warfarin.

In some embodiments, the recombinant hsNPP1 or hsNPP3 and additional therapeutic agent are administered separately and are administered concurrently or sequentially. In some embodiments, the recombinant hsNPP1 or hsNPP3 is administered prior to administration of the additional therapeutic agent. In some embodiments, the recombinant hsNPP1 or hsNPP3 is administered after administration of the additional therapeutic agent. In other embodiments, the recombinant hsNPP1 or hsNPP3 and additional therapeutic agent are administered together.

### Nucleic Acid Administration and Therapy

Viral Vectors for In Vivo Expression of ENPP1 and ENPP3

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically, the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein. Hence, genetic material such as a polynucleotide comprising an NPP1 or an NPP3 sequence can be introduced to a mammal in order to treat VSMC proliferation.

Certain modified viruses are often used as vectors to carry a coding sequence because after administration to a mammal, a virus infects a cell and expresses the encoded protein. Modified viruses useful according to the disclosure are derived from viruses which include, for example: parvovirus, picornavirus, pseudorabies virus, hepatitis virus A, B or C, papillomavirus, papovavirus (such as polyoma and SV40) or herpes virus (such as Epstein-Barr Virus, Varicella Zoster Virus, Cytomegalovirus, Herpes Zoster and Herpes Simplex Virus types 1 and 2), an RNA virus or a retrovirus, such as the Moloney murine leukemia virus or a lentivirus (i.e. derived from Human Immunodeficiency Virus, Feline Immunodeficiency Virus, equine infectious anemia virus, etc.). Among DNA viruses useful according to the disclosure are: Adeno-associated viruses adenoviruses, Alphaviruses, and Lentiviruses.

A viral vector is generally administered by injection, most often intravenously (by IV) directly into the body, or directly into a specific tissue, where it is taken up by individual cells. Alternately, a viral vector may be administered by contacting the viral vector ex vivo with a sample of the patient's cells, thereby allowing the viral vector to infect the cells, and cells containing the vector are then returned to the patient. Once the viral vector is delivered, the coding sequence expressed and results in a functioning protein. Generally, the infection and transduction of cells by viral vectors occur by a series of sequential events as follows: interaction of the viral capsid with receptors on the surface of the target cell, internalization by endocytosis, intracellular trafficking through the endocytic/proteasomal compartment, endosomal escape, nuclear import, virion uncoating, and viral DNA double-strand conversion that leads to the transcription and expression of the recombinant coding sequence interest. (Colella et al., Mol Ther Methods Clin Dev. 2017 Dec. 1; 8:87-104).

### Adeno-Associated Viral Vectors According to the Disclosure

AAV refers to viruses belonging to the genus Dependovirus of the Parvoviridae family. The AAV genome is approximately 4.7 kilobases long and is composed of linear single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding non-structural replication (Rep) proteins required for the AAV life cycle. The cap frame contains overlapping nucleotide sequences of structural VP capsid proteins: VP1, VP2 and VP3, which interact together to form a capsid of an icosahedral symmetry.

The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex. Following wild type AAV infection in mammalian cells the rep genes (i.e. Rep78 and Rep52) are expressed from the P5 promoter and the P19 promoter, respectively, and both Rep proteins have a function in the replication of the viral genome. A splicing event in the rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells.

Because the patient's cells lack the rep and cap genes, as well as the accessory function genes, the rAAV cannot further replicate and package their genomes. Moreover, without a source of 5 rep and cap genes, wtAAV cannot be formed in the patient's cells.

The AAV vector typically lacks rep and cap frames. Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In one embodiment, the disclosure relates to an adeno-associated viral (AAV) expression vector comprising a sequence encoding mammal ENPP1 or mammal ENPP3, and upon administration to a mammal the vector expresses an ENPP1 or ENPP3 precursor in a cell, the precursor including an Azurocidin signal peptide fused at its carboxy terminus to the amino terminus of ENPP1 or ENPP3. The ENPP1 or ENPP3 precursor may include a stabilizing domain, such as an IgG Fc region or human albumin. Upon secretion of the precursor from the cell, the signal peptide is cleaved off and enzymatically active soluble mammal ENPP1 or ENPP3 is provided extracellularly.

An AAV expression vector may include an expression cassette comprising a transcriptional regulatory region operatively linked to a nucleotide sequence comprising a transcriptional regulatory region operatively linked to a recombinant nucleic acid sequence encoding a polypeptide comprising an Azurocidin signal peptide sequence and an ectonucleotide pyrophosphatase/phosphodiesterase (ENPP1) polypeptide sequence.

In some embodiments, the expression cassette comprises a promoter and enhancer, the Kozak sequence GCCACCATGG, a nucleotide sequence encoding mammal NPP1 protein or a nucleotide sequence encoding mammal NPP3 protein, other suitable regulatory elements and a polyadenylation signal.

In some embodiments, the AAV recombinant genome of the AAV vector according to the disclosure lacks the rep open reading frame and/or the cap open reading frame.

The AAV vector according to the disclosure comprises a capsid from any serotype. In general, the AAV serotypes have genomic sequences of significant homology at the amino acid and the nucleic acid levels, provide an identical set of genetic functions, and replicate and assemble through practically identical mechanisms. In particular, the AAV of the present disclosure may belong to the serotype 1 of AAV (AAV1), AAV2, AAV3 (including types 3A and 3B), AAV4, AAV5, AAV6, AAV7, AAV8, AAV9, AAV10, AAVrh10, AAV11, avian AAV, bovine AAV, canine AAV, equine AAV, or ovine AAV.

Examples of the sequences of the genome of the different AAV serotypes may be found in the literature or in public databases such as GenBank. For example, GenBank accession numbers NC_001401.2 (AAV2), NC_001829.1 (AAV4), NC_006152.1 (AAV5), AF028704.1 (AAV6), NC_006260.1 (AAV7), NC_006261.1 (AAV8), AX753250.1 (AAV9) and AX753362.1 (AAV10).

In some embodiments, the adeno-associated viral vector according to the disclosure comprises a capsid derived from a serotype selected from the group consisting of the AAV2, AAV5, AAV7, AAV8, AAV9, AAV10 and AAVrh10 serotypes. In another embodiment, the serotype of the AAV is AAV8. If the viral vector comprises sequences encoding the capsid proteins, these may be modified so as to comprise an exogenous sequence to direct the AAV to a particular cell type or types, or to increase the efficiency of delivery of the targeted vector to a cell, or to facilitate purification or detection of the AAV, or to reduce the host response.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal a-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA 5 encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like. Also included in the disclosure is a method of treating a mammal having a disease or disorder in an amount effective to provide a therapeutic effect.

The method comprises administering to the mammal an rAAV vector encoding the polypeptide of interest. Preferably, the mammal is a human. Typically, the number of viral vector genomes/mammal which are administered in a single injection ranges from about 1×108 to about 5×1016. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹⁰ to about 5×10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, 15 to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal

The published application, US 2017/0290926—Smith et al., the contents of which are incorporated by reference in their entirety herein, describes in detail the process by which AAV vectors are generated, delivered and administered.

### RNA Based In Vivo Expression of ENPP1 and ENPP3 Polypeptides

The present disclosure provides compositions and methods for the production and delivery of recombinant double-stranded RNA molecules (dsRNA that encode ENPP1 or ENPP3 polypeptides described herein. The double stranded RNA particle (dsRP) can contain a dsRNA molecule enclosed in a capsid or coat protein. The dsRNA molecule can be a viral genome or portion of a genome, which can be derived from a wild-type viral genome. The RNA molecule can encode an RNA-dependent RNA polymerase (RDRP) and a polyprotein that forms at least part of a capsid or coat protein. The RNA molecule can also contain an RNA sub-sequence that encodes an ENPP1 or ENPP3 polypeptides that are translated by the cellular components of a host cell. When the dsRP is transfected into a host cell the sub-sequence can be translated by the cellular machinery of the host cell to produce the ENPP1 or ENPP3 polypeptides.

In another aspect the disclosure provides a method of producing a protein product in a host cell. The method includes transfecting a host cell with a dsRP having a recombinant double-stranded RNA molecule (dsRNA) and a capsid or coat protein. The RNA molecule can encode an RNA-dependent RNA polymerase and a polyprotein that forms at least part of the capsid or coat protein, and the dsRP can be able to replicate in the host cell. The RNA molecule has at least one RNA sub-sequence that encodes ENPP1 or ENPP3 polypeptides that is translated by cellular components of the host cell.

In another aspect the disclosure provides an RNA molecule translatable by a host cell. The RNA molecule can be any RNA molecule that encodes the ENPP1 or ENPP3 polypeptides described herein. In one embodiment the RNA molecule encodes an RNA-dependent RNA polymerase and a polyprotein that forms at least part of a capsid or coat protein of a dsRP and, optionally, can have at least one sub-sequence of RNA that encodes an additional protein product.

Production of dsRP

A dsRP of the disclosure can also be produced by presenting to a host cell a plasmid or other DNA molecule encoding a dsRP of the disclosure or encoding the genes of the dsRP. The plasmid or DNA molecule containing nucleotide sequences encoding desired protein such as ENPP1 or ENPP3 polypeptide is then transfected into the host cell and the host cell begins producing the dsRP of the disclosure. The dsRP can also be produced in the host cell by presenting to the host cell an RNA molecule encoding the genes of the dsRP. The RNA molecule can be (+)-strand RNA.

Once the dsRP of the disclosure has been presented to the host cell (or a plasmid encoding the genes of the dsRP of the disclosure, or an RNA molecule encoding the genes of the dsRP), the dsRP will be produced within the host cell using the cellular components of the host cell. The dsRP of the disclosure is therefore self-sustaining within the host cell and is propagated within the host cell. The host cell can be any suitable host cell such as, for example, a eukaryotic cell, a mammalian cell, a fungal cell, a bacterial cell, an insect cell, or a yeast cell. The host cell can propagate a recombinant dsRP after a recombinant dsRNA molecule of the disclosure or a DNA molecule encoding a dsRP of the disclosure is presented to and taken up by the host cell.

Methods of Producing a dsRNA Virus or dsRP

The disclosure also provides methods of producing a dsRP of the disclosure. A double-stranded or single-stranded RNA or DNA molecule can be presented to a host cell. The amplification of the dsRNA molecules in the host cell utilizes the natural production and assembly processes already present in many types of host cells (e.g., yeast). The disclosure can thus be applied by presenting to a host cell a single-stranded or double-stranded RNA or DNA molecule of the disclosure, which is taken up by the host cell and is utilized to produce the recombinant dsRP and protein or peptide encoded by the RNA sub-sequence using the host cell's cellular components. The disclosure can also be applied by providing to the host cell a linear or circular DNA molecule (e.g., a plasmid or vector) containing one or more sequences coding for an RNA-dependent RNA polymerase, a polyprotein that forms at least part of the capsid or coat protein of the dsRP, and a sub-sequence encoding the protein of interest such as ENPP1 or ENPP3 polypeptides as disclosed herein.

The presentation of a dsRNA or ssRNA molecule of the disclosure can be performed in any suitable way such as, for example, by presenting an RNA molecule of the disclosure directly to the host cell as “naked” or unmodified single-stranded or double-stranded RNA. The RNA molecule can be transfected (or transformed) into a yeast, bacterial, or mammalian host cell by any suitable method, for example by electroporation, exposure of the host cell to calcium phosphate, or by the production of liposomes that fuse with the cell membrane and deposit the viral sequence inside. It can also be performed by a specific mechanism of direct introduction of dsRNA from killer viruses or heterologous dsRNA into the host cell. This step can be optimized using a reporter system, such as red fluorescent protein (RFP), or by targeting a specific constitutive gene transcript within the host cell genome. This can be done by using a target with an obvious phenotype or by monitoring by quantitative reverse transcriptase PCR (RT-PCR).

In some embodiments a DNA molecule (e.g., a plasmid or other vector) that encodes an RNA molecule of the disclosure is introduced into the host cell. The DNA molecule can contain a sequence coding for the RNA molecule of a dsRP of the disclosure. The DNA molecule can code for an entire genome of the dsRP, or a portion thereof. The DNA molecule can further code for the at least one sub-sequence of RNA that produces the additional (heterologous) protein product. The DNA sequence can also code for gag protein or gag-pol protein, and as well as any necessary or desirable promoters or other sequences supporting the expression and purpose of the molecule. The DNA molecule can be a linear DNA, a circular DNA, a plasmid, a yeast artificial chromosome, or may take another form convenient for the specific application.

In one embodiment the DNA molecule can further comprise T7 ends for producing concatamers and hairpin structures, thus allowing for propagation of the virus or dsRP sequence in the host cell. The DNA molecule can be transfected or transformed into the host cell and then, using the host cellular machinery, transcribed and thus provide the dsRNA molecule having the at least one sub-sequence of RNA to the host cell. The host cell can then produce the encoded desired ENPP1 or ENPP3 polypeptide. The dsRNA can be packaged in the same manner that a wild-type virus would be, using the host cell's metabolic processes and machinery. The ENPP1 or ENPP3 polypeptide is also produced using the host cell's metabolic processes and cellular components.

The patent, U.S. Ser. No. 10/266,834 by Brown et al., the contents of which are incorporated by reference in their entirety herein, describes in detail the process by which dsRNA particles that encode polypeptides are generated, delivered and administered.

ENPP1 Coated Stents and ENPP3 Coated Stents

Stents are typically elongated structures used to keep open lumens (e.g., openings in the body) found in various parts of the body so that the parts of the body containing those lumens may function properly. Stents are often used in the treatment of atherosclerosis, a disease of the vascular system in which arteries become partially, and sometimes completely, occluded with substances that may include lipids, cholesterol, calcium, and various types of cells, such as smooth muscle cells and platelets.

Stents located within any lumen in the body may not always prevent partial or complete restenosis. In particular, stents do not always prevent the re-narrowing of an artery following Percutaneous transluminal angioplasty (PTA). In some cases, the introduction and presence of the stent itself in the artery or vein can create regions of trauma or tissue injury such as, e.g., tears in the inner lining of the artery, called the endothelium requiring further surgeries post stent placement.

It is believed that such trauma or tissue injury can trigger migration of vascular smooth muscle cells, which are usually separated from the arterial lumen by the endothelium, into the arterial lumen, where they proliferate to create a mass of cells that may, in a matter of days or weeks, occlude the artery. Such re-occlusion, which is sometimes seen after PTA, is an example of restenosis. Coating a stent with therapeutic agent such as ENPP1 agent or ENPP3 agent is expected to prevent and/or reduce vascular smooth muscle cell proliferation which in return reduces the occurrence of or treats restenosis.

In some embodiments, the patient is need of surgery and/or has tissue injury due to the presence of a prior implanted non-eluting stent.

In some embodiments, the patient is need of surgery and/or has tissue injury due to the presence of a prior implanted eluting stent that elutes therapeutic agents other than ENPP1 agent or ENPP3 agent.

In some embodiments, the prior stent that had caused the tissue injury is removed and replaced with ENPP1 agent coated stent.

In some embodiments, the prior stent that had caused the tissue injury is removed and replaced with ENPP3 agent coated stent.

In some embodiments, the prior stent that had caused the tissue injury is not removed and the ENPP1 agent coated stent is implanted adjacent to the prior stent.

In some embodiments, the prior stent that had caused the tissue injury is not removed and the ENPP3 agent coated stent is implanted adjacent to the prior stent.

ENPP1 or ENPP3 coated stents are typically hollow, cylindrical structures made from struts or interconnected filaments. Stents are usually implanted at their site of use in the body by attaching them in a compressed state to a catheter that is directed through the body to the site of stent use. Vascular stents are frequently used in blood vessels to open the vessel and provide improved blood flow. The stent can be expanded to a size which enables it to keep the lumen open by supporting the walls of the lumen once it is positioned at the desired site. Vascular stents can be collapsed to reduce their diameter so that the stent can be guided through a patient's arteries or veins to reach the site of deployment. Stents are typically either coupled to the outside of the balloon for expansion by the expanding balloon or are self-expanding upon removal of a restraint such as a wire or sleeve maintaining the stent in its collapsed state.

Vascular stents are often made of metal to provide the strength necessary to support the occluded arterial walls. Two of the preferred metals are Nitinol alloys of nickel and titanium, and stainless steel. Other materials that can be used in fabricating stents are ceramics, polymers, and plastics. The polymer may be a polymer having no functional groups. Alternatively, the polymer may be one having functional groups, but none that are reactive with the ENPP1 agent or ENPP3 agent. The polymer may include a biodegradable polymer. For example, the polymer may include a polymer selected from the group consisting of polyhydroxy acids, polyanhydrides, polyphosphazenes, polyalkylene oxalates, biodegradable polyamides, polyorthoesters, polyphosphoesters, polyorthocarbonates, and blends or copolymers thereof. The polymer may also include a biostable polymer, alone or in combination with a biodegradable polymer. For example, the polymer may include a polymer selected from the group consisting of polyurethanes, silicones, polyacrylates, polyesters, polyalkylene oxides, polyalcohols, polyolefins, polyvinyl chlorides, cellulose and its derivatives, fluorinated polymers, biostable polyamides, and blends or copolymers thereof.

The effect of different stent designs on the drug distribution pattern has been scrutinized in experimental studies and also tested in clinical trials (Hwang C W, Wu D, Edelman E R. 2001. Physiological transport forces govern drug distribution for stent-based delivery. Circulation, 104: 600-5; & Takebayashi H, Mintz G S, Carlier S G, et al. 2004. Nonuniform strut distribution correlates with more neointimal hyperplasia after Sirolimus-eluting stent implantation. Circulation, 110:3430-4). Although a large number of stent designs have been developed to date, only the multicellular design is currently most commonly used; they can be categorized into “closed cell” and “open cell” configurations (Rogers C D K. 2002. Drug-eluting stents: role of stent design, delivery vehicle, and drug selection. Rev Cardiovasc Med, 3(Suppl 5): S10-15). A closed cell stent has a uniform cell expansion and constant cell spacing when deployed in a curved vascular segment, which gives more uniform drug distribution (Rogers 2002). An open cell stent has a greater variation in the surface coverage between the inner and outer curvatures in the curved segment but gives better conformability to curved surface at the expense of less uniform drug distribution (Rogers 2002). The majority of current stents use a closed cell design. The optimal stent design for drug delivery would have a large stent surface area, a small cell gap, and minimal strut deformation after deployment while maintaining conformability, radial support, and flexibility to reach the complex coronary lesions. Several examples of the different geometrical stent structures are described in Paisal et al. (Muhammad Sufyan Amir Paisal et al 2017 IOP Conf. Ser.: Mater. Sci. Eng. 165 012003)

ENPP1 coated stents or ENPP3 coated stents are prepared by applying a coating composition comprising an effective amount of ENPP1 agent or ENPP3 agent respectively. The coating composition preferably includes an amount of the ENPP1 agent or ENPP3 agent that is sufficient to be therapeutically effective for inhibiting regrowth of plaque or inhibiting restenosis or preventing vascular smooth cell proliferation.

In one embodiment, the coating composition comprises from about 1 wt % to about 50 wt % ENPP1 polypeptide, based on the total weight of the coating composition. In another embodiment, the coating composition comprises from about 5 wt % to about 30 wt % ENPP1 polypeptide. In yet another embodiment, the coating composition comprises from about 10 wt % to about 20 wt % ENPP1 polypeptide.

In one embodiment, the coating composition comprises from about 1 wt % to about 50 wt % ENPP3 polypeptide, based on the total weight of the coating composition. In another embodiment, the coating composition comprises from about 5 wt % to about 30 wt % ENPP3 polypeptide. In yet another embodiment, the coating composition comprises from about 10 wt % to about 20 wt % ENPP3 polypeptide.

In one embodiment, the coating composition comprises from about 1 μg/ml to about 10 mg/ml of ENPP1 polypeptide. In another embodiment, the coating composition comprises from about 100 μg/ml to 5 mg/ml ENPP1 polypeptide. In yet another embodiment, the coating composition comprises from about 500 μg/ml to about 2 mg/ml ENPP1 polypeptide.

In a related embodiment, the ENPP1 polypeptide of the coating composition is ENPP1-Fc.

In a related embodiment, the ENPP1 polypeptide of the coating composition is ENPP1-Albumin.

In one embodiment, the coating composition comprises from about 1 μg/ml to about 10 mg/ml of ENPP3 polypeptide. In another embodiment, the coating composition comprises from about 100 μg/ml to 5 mg/ml ENPP3 polypeptide. In yet another embodiment, the coating composition comprises from about 500 μg/ml to about 2 mg/ml ENPP3 polypeptide.

In a related embodiment, the ENPP3 polypeptide of the coating composition is ENPP3-Fc.

In a related embodiment, the ENPP3 polypeptide of the coating composition is ENPP3-Albumin.

In one embodiment, the coating composition comprises from about 1 ng/μl to about 1000 μg/μl of ENPP1 mRNA. In another embodiment, the coating composition comprises from about 100 ng/μl to 10 μg/μl ENPP1 mRNA. In yet another embodiment, the coating composition comprises from about 50 ng/μl to about 5 μg/μl ENPP1 mRNA.

In one embodiment, the coating composition comprises from about 1 ng/μl to about 1000 μg/μl of ENPP1-Fc mRNA. In another embodiment, the coating composition comprises from about 100 ng/μl to 10 μg/μl ENPP1-Fc mRNA. In yet another embodiment, the coating composition comprises from about 50 ng/μl to about 5 μg/μl ENPP1-Fc mRNA.

In one embodiment, the coating composition comprises from about 1 ng/μl to about 1000 μg/μl of ENPP1-Albumin mRNA. In another embodiment, the coating composition comprises from about 100 ng/μl to 10 μg/μl ENPP1-Albumin mRNA. In yet another embodiment, the coating composition comprises from about 50 ng/μl to about 5 μg/μl ENPP1-Albumin mRNA.

In one embodiment, the coating composition comprises from about 1 ng/μl to about 1000 μg/μl of ENPP3 mRNA. In another embodiment, the coating composition comprises from about 100 ng/μl to 5 μg/μl ENPP3 mRNA. In yet another embodiment, the coating composition comprises from about 500 ng/μl to about 2 μg/μl ENPP3 mRNA.

In one embodiment, the coating composition comprises from about 1 ng/μl to about 1000 μg/μl of ENPP3-Fc mRNA. In another embodiment, the coating composition comprises from about 100 ng/μl to 5 μg/μl ENPP3-Fc mRNA. In yet another embodiment, the coating composition comprises from about 500 ng/μl to about 2 μg/μl ENPP3-Fc mRNA.

In one embodiment, the coating composition comprises from about 1 ng/μl to about 1000 μg/μl of ENPP3-Albumin mRNA. In another embodiment, the coating composition comprises from about 100 ng/μl to 5 μg/μl ENPP3-Albumin mRNA. In yet another embodiment, the coating composition comprises from about 500 ng/μl to about 2 μg/μl ENPP3-Albumin mRNA.

Stents may be coated with a substance, such as a biodegradable or biostable polymer, to improve the biocompatibility of the stent, making it less likely to cause an allergic or other immunological response in a patient. A coating substance may also add to the strength of the stent. Some known coating substances include organic acids, their derivatives, and synthetic polymers that are either biodegradable or biostable. Biostable coating substances do not degrade in the body, biodegradable coating substances can degrade in the body.

The coating composition comprises an effective amount of carrier which helps in the coating process to ensure that the therapeutic molecules such as ENPP1 agent or ENPP3 agent adhere to the stent surface and also facilitate in eluting the therapeutic agent into the body at the site of stent placement. The carrier could be a liquid carrier or a solid carrier. The coating composition may alternatively comprise more than one solid compound in a solid carrier. The coating composition may further comprise both a liquid carrier and a solid carrier. In a still further aspect, the coating composition may also comprise more than one type of nonpolymeric or polymeric compound in the carrier and may further comprise both a polymeric material and a nonpolymeric material in a solid or liquid carrier.

In another embodiment, two or more types of biodegradable compounds (polymers or non-polymers) may be blended together to obtain a liquid carrier for use in the coating composition. The biodegradable compounds can be liquids before they are mixed together, e.g., forming a homogeneous solution, mixture, or suspension. Alternatively, some of the biodegradable compounds may be solids before they are mixed with other liquid biodegradable compounds. The solid biodegradable compounds preferably dissolve when they are mixed with the liquid biodegradable compounds, resulting in a liquid carrier composition containing the different biodegradable compounds.

In another embodiment, the biodegradable carrier component of the coating composition is a solid, which dissolves when mixed with the biologically active component and any other components included in the coating composition.

The carrier could be a polymeric carrier. Some polymeric carriers are synthetic polymers. Examples of synthetic polymers that serve as reservoir matrices include but not limited to poly-n-butyl methacrylate, polyethylene-vinyl acetate, poly (lactide-co-Σ-caprolactone) copolymer, Fibrin, cellulose, Phosphorylcholine. Some eluting stent comprise porous 300 μm ceramic layer containing therapeutic molecule-loaded nanocavities. Examples of drug eluting stents, stent structures and stent designs can be found in Drug-Eluting Stent: A Review and Update, Vasc Health Risk Manag. 2005 December; 1(4): 263-276 and Modern Stents: Where Are We Going?, Rambam Maimonides Med J. 2020 April; 11(2): e0017.

The carriers in the coating composition may be either biodegradable or biostable. Biodegradable polymers are often used in synthetic biodegradable sutures. These polymers include polyhydroxy acids. Polyhydroxy acids suitable for use in the present invention include poly-L-lactic acids, poly-DL-lactic acids, polyglycolic acids, polylactides including homopolymers and copolymers of lactide (including lactides made from all stereo isomers of lactic acids, such as D-,L-lactic acid and meso lactic acid), polylactones, polycaprolactones, polyglycolides, polyparadioxanone, poly 1,4-dioxepan-2-one, poly 1,5-dioxepan-2-one, poly 6,6-dimethyl-1, 4-dioxan-2-one, polyhydroxyvalerate, polyhydroxybuterate, polytrimethylene carbonate polymers, and blends of the foregoing.

Polylactones suitable for use in the present invention include polycaprolactones such as poly(e-caprolactone), polyvalerolactones such as poly(d-valerolactone), and polybutyrolactones such as poly(butyrolactone). Other biodegradable polymers that can be used are polyanhydrides, polyphosphazenes, biodegradable polyamides such as synthetic polypeptides such as polylysine and polyaspartic acid, polyalkylene oxalates, polyorthoesters, polyphosphoesters, and polyorthocarbonates. Copolymers and blends of any of the listed polymers may be used. Polymer names that are identical except for the presence or absence of brackets represent the same polymers.

Biostable polymers suitable for use in the present invention include, but are not limited to polyurethanes, silicones such as polyalkyl siloxanes such as polydimethyl siloxane and polybutyl methacrylate, polyesters such as poly(ethylene terephthalate), polyalkylene oxides such as polyethylene oxide or polyethylene glycol, polyalcohols such as polyvinyl alcohols and polyethylene glycols, polyolefins such as poly-5 ethylene, polypropylene, poly(ethylene-propylene) rubber and natural rubber, polyvinyl chloride, cellulose and modified cellulose derivatives such as rayon, rayon-triacetate, cellulose acetate, cellulose acetate butyrate, cellophane, cellulose nitrate, cellulose propionate, cellulose ethers such as carboxymethyl cellulose and hydroxyalkyl celluloses, fluorinated polymers such as polytetrafluoroethylene (Teflon), and bio stable polyamides such as Nylon 66 and polycaprolactam. Fixed animal tissues such as glutaraldehyde fixed bovine pericardium can also be used. Polyesters and polyamides can be either biodegradable or biostable. Ester and amide bonds are susceptible to hydrolysis, which can contribute to biodegradation.

In some cases, the coating composition further comprises an effective amount of a non-polymeric carrier. The non-polymeric carrier can include one or more of fatty acid, biocompatible oil, or wax. Examples of non-polymeric biodegradable carriers include liquid oleic acid, vitamin E, peanut oil, and cottonseed oil, which are liquids that are both hydrophobic and biocompatible. In some cases, the nonpolymeric or polymeric carrier, can be a liquid at room and body temperature. In some cases, the nonpolymeric or polymeric carrier can be a solid at room and body temperature, or a solid at room temperature and a liquid at body temperature.

In another embodiment, the polymer solution can be formed into a film and the film then applied to the stent. Any of a variety of conventional methods of forming films can be used. For example, the polymer, ENPP1 agent or ENPP3 agent and solvent are preferably mixed into solution and then poured onto a smooth, flat surface such that a coating film is formed after the solution is dried to remove the solvent. The film can then be cut to fit the stent on which it is to be used. The film may then be mounted, such as by wrapping, on the outer surface of a stent.

In another embodiment, the coated stent is prepared by spraying the stent with the liquid carrier comprising the therapeutic agent such as ENPP1 agent or ENPP3 agent resulting in a coating of uniform thickness on the struts of the stent. In another embodiment, the stent may be dip coated or immersed in the coating solution comprising carrier and therapeutic agent, such that the solution completely coats the struts of the stent. Alternatively, the stent may be painted with the coating solution comprising carrier and therapeutic agent, such as with a paint brush. In each of these coating applications, the entirety of both the outer and inner surfaces of the stent are preferably coated, although only portions of either or both surfaces may be coated in some embodiments.

As discussed above, the coating composition comprises a bioactive component and a biodegradable carrier component. Preferably, the coating composition comprises from 0.1% to 100% by weight of a biologically active component and from 1% to 99% by weight of a biodegradable carrier component. More preferably, the coating composition comprises from 0.1% to 50% by weight of a biologically active component and from 50% to 99.9% by weight of a biodegradable carrier component. The coating composition can be prepared in a number of ways including by simply mixing the bioactive component and the carrier component together to form a mixture, e.g., a solution or suspension. Alternatively, the bioactive component and the carrier component together are mixed in a suitable solvent, the coating is applied to the stent, and the solvent is removed. Preferably the coating composition is applied to the stent in its expanded state.

In addition to stents, examples of other medical devices that can be coated in accordance with aspects of the inventions disclosed herein include catheters, heart valves, pacemaker leads, annuloplasty rings and other medical implants. In other specific embodiments, coated angioplasty balloons and other coated medical devices can also comprise one of the coating compositions disclosed herein. However, stents are preferred. The coating composition may be applied to the stent (or other medical device) by any number of ways, e.g, by spraying the coating composition onto the stent, by immersing the stent in the coating composition, or by painting the stent with the coating composition. Preferably, a stent is coated in its expanded (i.e., enlarged diameter) form so that a sufficient amount of the coating composition will be applied to coat the entire surface of the expanded stent. When the stent is immersed in the coating composition, the excess coating composition on the surface of the stent may be removed, such as by brushing off the excess coating composition with a paint brush. In each of these coating applications, preferably both the outer and inner surfaces of the stent are coated.

The coating compositions described herein preferably remain on a stent, partially or in substantial part, after the stent has been introduced to the body, for at least several days, for several weeks and more preferably for several months thereby slowly releasing the therapeutic agents such as ENPP1 agent or ENPP3 agent into the blood stream.

### Pharmaceutical Compositions and Formulations

The disclosure provides pharmaceutical compositions comprising a polypeptide of the disclosure within the methods described herein. Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation. In certain embodiments, administration of the compound of the disclosure to a subject elevates the subject's plasma PPi to a level that is close to normal, where a normal level of PPi in mammals is 1-3 μM. “Close to normal” refers to 0 to 1.2 μM or 0-40% below or above normal, 30 nM to 0.9 μM or 1-30% 15 below or above normal, 0 to 0.6 μM or 0-20% below or above normal, or 0 to 0.3 μM or 0-10% below or above normal.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure is administered at an appropriate time interval of every 2 days, or every 4 days, or every week or every month so as to achieve a continuous level of plasma PPi that is either close to the normal (1-3 μM) level or above (30-50% higher than) normal levels of PPi. Therapeutic dosage of the polypeptides of the disclosure may also be determined based on half-life or the rate at which the therapeutic polypeptide is cleared out of the body. The polypeptide according to the disclosure is administered at appropriate time intervals of either every 2 days, or every 4 days, every week or every month so as to achieve a constant level of enzymatic activity of ENPP1 or ENPP3 polypeptides.

For example, several divided doses may be administered daily, or the dose may be proportionally reduced as indicated by the exigencies of the therapeutic situation. A non-limiting example of an effective dose range for a therapeutic compound of the disclosure is from about 0.01 and 50 mg/kg of body weight/per day. In certain embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient. Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

“Parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### EXAMPLES

The present disclosure is further exemplified by the following examples. The examples are for illustrative purpose only and are not intended, nor should they be construed as limiting the disclosure in any manner.

### Example 1: Efficacy of ENPP1-Fc Fusion Protein in In-Stent Restenosis Model

The efficacy of an ENPP1-Fc fusion protein was evaluated in large animal model of peripheral vascular injury—specifically, in-stent restenosis lesions in the peripheral vasculature of domestic (Yorkshire) swine. Therapeutic effects of the ENPP1-Fc fusion protein were assessed with respect to the ability to inhibit stenosis after angioplasty in previously injured and stented peripheral arteries of Yorkshire swine.

Four peripheral arterial sites were created for induction of neointimal response in each animal; one site was selected in each of four arteries (bilateral profunda and superficial femoral arteries).

All target sites were injured on Day 0 to create the in-stent restenosis model, 10 days prior to the first dose of ENPP1-Fc or a vehicle only control, and 14 days before repeat injury. The four peripheral artery sites were mapped using quantitative vascular angiography (QVA) in order to select the treatment site and correctly sized balloon and stent. The injury was created by overstretch of the artery with a standard angioplasty balloon catheter at a target 130% overstretch; three inflations were performed. Immediately following injury, a bare metal stent was deployed. Peripheral stents were self-expandable, targeting approximately a 120% overstretch.

ENPP1-Fc treatment occurred systemically starting on Day 10 and was dosed every 4 days subcutaneously until termination. On Day 14, all vessels were assessed by angiography and Optical Coherence Tomography (OCT). Then the previously injured and stented artery sites were subjected to a re-injury event consisting of overstretch of the artery with a single inflation of a standard angioplasty balloon catheter at the same pressure/diameter as the original pre-stent injury was done (130% of the baseline reference diameter). Following re-injury interventions, final post-procedural angiography and OCT were also recorded for select peripheral sites.

Four weeks following the re-injury event on Day 14, arteries underwent repeat imaging with angiography and endovascular imaging (OCT). The treated peripheral segments were explanted and stored in 10% neutral buffered formalin.

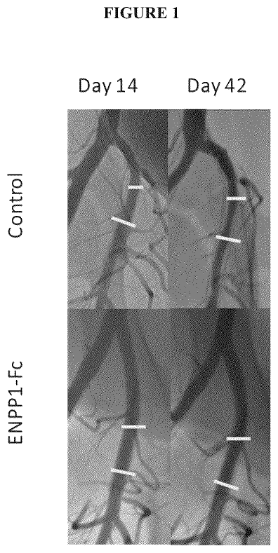

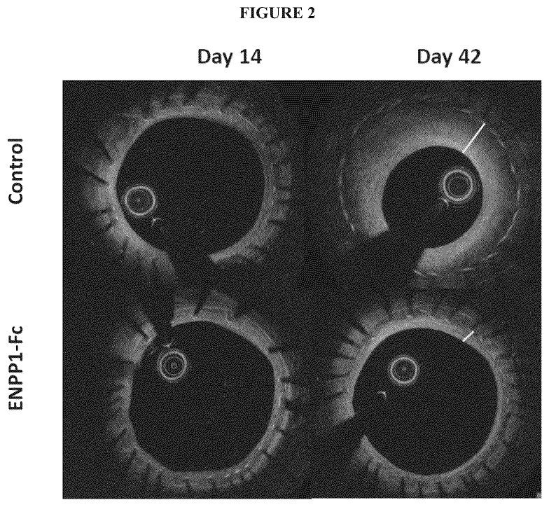

As shown in FIG. 1, angiography revealed a pronounced narrowing of the profunda at day 42 relative to the morphology of the vessel at day 14 in animals given the vehicle control. By contrast, in animals treated with ENPP1-Fc little visible change in profunda morphology was observed between day 14 and day 42. Similarly, as measured by OCT, pronounced intimal thickening was observed within the profunda at day 42 relative to the morphology of the vessel at day 14 in animals treated with the vehicle control. By contrast, little visible intimal thickening was observed between day 14 and day 42 in the profunda of animals treated with ENPP1-Fc (FIG. 2).

Tables 1 and 2 (below) summarizes the mean OCT values in all profunda arteries by treatment group.

TABLE 1

| | Day 14 Re-Injury, Profunda | | | | |
|---|---|---|---|---|---|
| | Lumen | Stent Area | Neointimal | Neointimal | % Area of |
| | Area (mm²) | (mm²) | Thickness (mm) | Area (mm²) | Stenosis |
| ENPP1- | 12.10 ± 1.09 | 14.59 ± 1.24 | 0.19 ± 0.04 | 2.49 ± 0.52 | 17 ± 3 |
| Fc | | | | | |
| Control | 10.82 ± 1.06 | 13.60 ± 0.82 | 0.23 ± 0.04 | 2.78 ± 0.40 | 21 ± 4 |

TABLE 2

| | Day 42 Termination, Profunda | | | | |
|---|---|---|---|---|---|
| | Lumen | Stent Area | Neointimal | Neointimal | % Area of |
| | Area (mm²) | (mm²) | Thickness (mm) | Area (mm²) | Stenosis |
| ENPP1- | 12.95 ± 0.70 | 16.47 ± 0.89 | 0.27 ± 0.08 | 3.52 ± 1.07 | 21 ± 5 |
| Fc | | | | | |
| Control | 9.51 ± 2.24 | 14.60 ± 1.40 | 0.45 ± 0.16 | 5.10 ± 1.34 | 35 ± 11 |

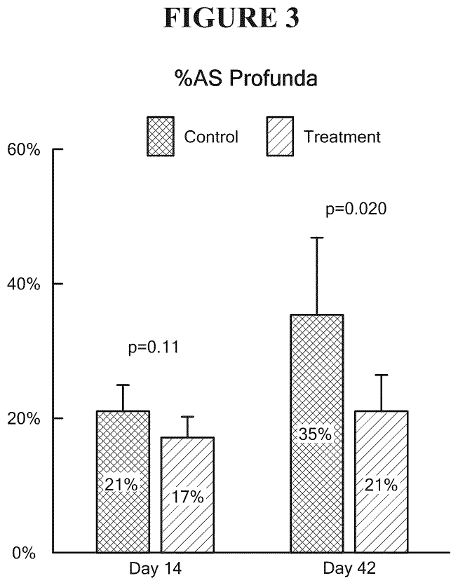

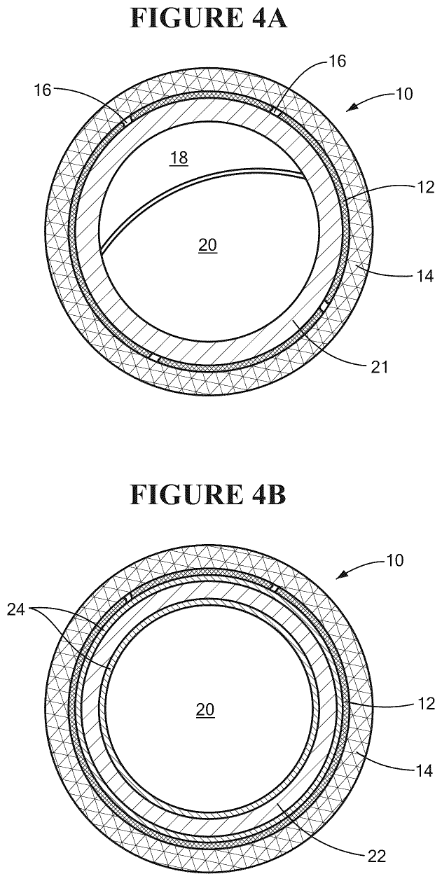

As set forth in the Table, the profunda arteries of animals treated with ENPP1-Fc had a higher lumen area at day 42 compared to the vehicle control group. The stent area was similar between both groups. Neointimal thickness and neointimal area were also reduced at day 42 in animals treated with ENPP1-Fc relative to the vehicle control animals. In additional, animals treated with ENPP1-Fc had a markedly lower % area of stenosis as compared to the vehicle control group (see FIG. 3). These data indicate that ENPP1 polypeptides are useful for, among other things, inhibiting the intimal thickening associated with injury of and/or to peripheral arteries.

### Example 2: Efficacy of ENPP3-Fc Fusion Protein in In-Stent Restenosis Model

The efficacy of an ENPP3-Fc fusion protein is evaluated in large animal model of peripheral vascular injury—specifically, in-stent restenosis lesions in the peripheral vasculature of domestic (Yorkshire) swine. Therapeutic effects of the ENPP3-Fc fusion protein are assessed with respect to the ability to inhibit stenosis after angioplasty in previously injured and stented peripheral arteries of Yorkshire swine.

Four peripheral arterial sites are created for induction of neointimal response in each animal as described in Example 1. All target sites are injured on Day 0 to create the in-stent restenosis model, 10 days prior to the first dose of ENPP3-Fc or a vehicle only control, and 14 days before repeat injury. The four peripheral artery sites are mapped using quantitative vascular angiography (QVA) in order to select the treatment site and correctly sized balloon and stent. The injury is created by overstretch of the artery with a standard angioplasty balloon catheter at a target 130% overstretch; three inflations are performed. Immediately following injury, a bare metal stent is deployed. Peripheral stents are self-expandable, targeting approximately a 120% overstretch.

ENPP3-Fc treatment is initiated systemically starting on Day 10 and is dosed every 4 days subcutaneously until termination. On Day 14, all vessels are assessed by angiography and Optical Coherence Tomography (OCT). Then the previously injured and stented artery sites are subjected to a re-injury event consisting of overstretch of the artery with a single inflation of a standard angioplasty balloon catheter at the same pressure/diameter as the original pre-stent injury (130% of the baseline reference diameter). Following re-injury interventions, final post-procedural angiography and OCT are recorded for select peripheral sites.

Four weeks following the re-injury event on Day 14, arteries underwent repeat imaging with angiography and endovascular imaging (OCT). The treated peripheral segments are explanted and stored in 10% neutral buffered formalin.

Without being bound to any one theory, it is expected that animals treated with ENPP3-Fc would exhibit lower % area of stenosis as compared to the vehicle control group. It is expected that ENPP3 polypeptides would be useful for, among other things, inhibiting the intimal thickening associated with injury of and/or to peripheral arteries.

### Example 3—ENPP1 Eluting Coated Stent for the Treatment of Atherosclerotic Peripheral Blood Vessels

In this example the ability of ENPP1 polypeptides (ENPP1 or ENPP1-Fc or ENPP1-Albumin) coated stents to inhibit neointima formation and inflammation in peripheral arteries thereby reducing thrombosis and/or vessel occlusion.

Without being bound to any one theory, it is expected that inducing the overexpression of ENPP1 polypeptides (ENPP1 or ENPP1-Fc or ENPP1-Ablumin) at the site of the implanted stent in the peripheral arteries would result in one or more (i) a decrease in platelet activation, (ii) a reduction in restenosis and inflammatory responses, and (iii) a decrease in VSMC proliferation, following stent implantation. This therapy is based on the delivery of ENPP1 mRNA (or ENPP1-Fc mRNA or ENPP1-Albumin mRNA) to the endothelial cells, which then in turn express the ENPP1 protein at the site of the stent implant after mRNA translation.

Production of ENPP1 mRNA

pcDNA 3.3 plasmid (Eurofins Genomics GmbH, Ebersberg, Germany) containing ENPP1 DNA templates is amplified using the HotStar HiFidelity Polymerase Kit (Qiagen, Hilden, Germany) according to the manufacturer's instructions. The PCR product (PCR cycler: Eppendorf, Wesseling, Germany) is purified with the Qiaquick PCR Purification Kit (Qiagen). In vitro transcribed mRNA is generated with the MEGAscript1 T7 Kit (Ambion, Glasgow, Scotland) according to the manufacturer's instructions.

To modify the mRNA a 3″-O-Mem7 G(5′)ppp(5′)G RNA Cap Structure Analog (New England Biolabs, Frankfurt, Germany) is added to the reaction as well as pseudouridine-5′-triphosphate and 5-methylcytidine-5′-triphosphate (TriLink Biotech, San Diego, CA, USA), which are substituted for UTP and CTP, respectively. For RNase inhibition 1 μl of RNase inhibitor (Thermo Scientific, Waltham) is added per reaction. The in vitro transcribed mRNA is then purified with the RNeasy Kit (Qiagen). The purified mRNA is dephosphorylated using the Antarctic Phosphatase Kit (New England Biolabs) and once again purified with the RNeasy Kit (Qiagen). The same procedure is repeated to generate enhanced green fluorescent protein (eGFP) mRNA using eGFP DNA. (Avci-Adali M, Behring A, Keller T, Krajewski S, Schlensak C, Wendel HP (2014) Optimized conditions for successful transfection of human endothelial cells with in vitro synthesized and modified mRNA for induction of protein expression. J Blot Eng 8: 8).

The functionality of the generated ENPP1 mRNA is validated by measuring free phosphate after hydrolysis of ATP by transfected HEK293 cells. ENPP1 mRNA transfected HEK293 cells are incubated with 20 μM ATP (möLab, Langenfeld, Germany) or PBS as control for 10 min at 37° C. on a shaking platform (Polymax 1040, Heidolph, Schwabach, Germany). The ATP substrate degrades over time in the presence of ENPP1, with the accumulation of the enzymatic product AMP. Using varying concentrations of ATP substrate, the initial rate velocities for ENPP1 are derived in the presence of ATP, and the data is fit to a curve to derive the enzymatic rate constants.

### Stent Coating

In order to develop a bioactive stent coating, which allows local delivery of ENPP1mRNA and transfection of endothelial cells in vivo, the generated ENPP1 mRNA is first coated on thermanox plastic slides. The stent coating is thus simulated using thermanox plastic slides (Nunc, Thermo scientific, USA). First, 100.000 HEK293 cells per well are seeded on a 12-well plate.

After 24 hours, 2 μl Lipofectamin as well as 10 μg ENPP1 mRNA are mixed with 50 μl Opti-MEM and incubated at room temperature for 20 min. Meanwhile, 10 μl from a polylactic-co-glycolic-acid (PLGA) (Evoniks, Darmstadt) stock solution (20 mg/ml)) is diluted in 990 μl ethyl acetate (final concentration 200 μg/ml). Then 200 μl of the PLGA solution are mixed with the transfection complexes.

The thermanox slides are coated with the solution in a step-by-step approach at room temperature. eGFP mRNA and sterilized water are used as controls. The HEK293 cells are supplied with a new medium before the dried slides are plated face down onto the cells. The cells are incubated with the slides at 37° C. and 5% CO₂ for 24 hrs, 48 hrs and 72 hrs and then analyzed using a FACScan cytometer.

The expression of ENPP1 of HEK293 cells is measured using flow cytometry. The ENPP1 coated thermonox slide exposed cells and control cells are stained with anti-ENPP1-fluorescein isothiocyanate (FITC) antibody. Flow cytometric analysis of the HEK293 cells after incubation with the ENPP1mRNA/PLGA covered thermanox slides are expected to show that the ENPP1 mRNA is released from the PLGA coating, whereby increase in ENPP1 expression is expected to be detectable after 24 hours, 48 hours and 72 hours post exposure to slides.

Compared to control HEK293 cells, (which were exposed thermonox slides coated with Lipofectamine alone) 0.5-1 μg of the ENPP1 mRNA is expected to be sufficient to induce increase of the ENPP1 protein expression in HEK cells exposed to ENPP1 mRNA coated thermonox slides even after 24 hours of exposure.

Without being bound to any one theory, it is proposed herein that the ENPP1 expressed at the site of the stent implant is expected to prevent intimal proliferation and reduce platelet occlusion in peripheral arteries.

### Example 4—Preparation and Implantation of ENPP1 Coated Stent for the Treatment of Atherosclerotic Peripheral Blood Vessels

In this example, a juvenile pig animal model is used for implanting the ENPP1-coated stent to determine the efficacy of an ENPP1 coated stent to inhibit neointima formation, restenosis and inflammation. An ENPP1 agent coated stent is prepared and then implanted in peripheral artery. Four peripheral arterial sites are created for induction of neointimal response in each animal; one site was selected in each of four arteries (bilateral profunda and superficial femoral arteries) as described in Example 1.

Preparation of ENPP1 Coated Stent

Any stent is amenable to be coated with ENPP1 agent. Common examples of commercial sources that sell stents for use include Abbot, Boston Scientific, Medtronic, Alvimedica, Lepu Medical Technology, Cordis, Balton or Biotronik. Peripheral arterial stents are shorter in length (12-18 mm) with a diameter range from 5-8 mm are commonly used for placement in iliac and femoral arteries. Henry et al. describes in detail the different types of stent lengths and diameters available for peripheral arteries (Henry et al., Tex Heart Inst J 2000; 27(2): 119-126.)

For example, a plain stent such as a bare metal stent can be converted to ENPP1 coated eluting stent by placing a polymeric film comprising ENPP1 mRNA inside the stent or by spraying a polymeric or nonpolymeric solution comprising ENPP1 mRNA or ENPP1 polypeptide on to the stent surface.

Some examples of ENPP1 polymeric film are shown below, the ENPP1 polymeric film can be placed inside stents to create ENPP1 coated eluting stents. Optionally nonpolymeric carrier such as Vitamin E, Vitamin E acetate, Vitamin E succinate, oleic acid, peanut oil and cottonseed oil can be added to the solution improve the stability of ENPP1 agent in the polymeric film
(a) ENPP1 agent coating composition (A)—10 mg PCL (poly caprolactone) polymer and 100 μg ENPP1 mRNA (or ENPP1-Fc mRNA or ENPP1-Albumin mRNA) are dissolved in sterile double distilled water at room temperature. The solution is poured onto a glass plate and the solvent is allowed to evaporate for 12-24 hours. After almost complete removal of the solvent, the ENPP1-loaded PCL film is removed from the glass plate and is cut to 1.5 cm by 1.5 cm size which is then further trimmed to fit the size of the stent. The ENPP1 mRNA (or ENPP1-Fc mRNA or ENPP1-Albumin mRNA) comprising polymeric film is then mounted on the stainless stent. The same process can be repeated for preparing a stent coated with a vector expressing ENPP1 polypeptide (ENPP1 or ENPP1-Fc or ENPP1-Albumin) by using 50 μg of vector DNA.
(b) ENPP1 agent coating composition (B)—10 mg EVA (ethylene-vinyl acetate) polymer and 100 μg ENPP1 mRNA (or ENPP1-Fc mRNA or ENPP1-Albumin mRNA) are dissolved in sterile double distilled water at room temperature. The solution is poured onto a glass plate and the solvent is allowed to evaporate for 12-24 hours. After almost complete removal of the solvent, the ENPP1-mRNA (or ENPP1-Fc mRNA or ENPP1-Albumin mRNA) loaded EVA film is removed from the glass plate and was cut to 1.5 cm by 1.5 cm size. The ENPP1 mRNA (or ENPP1-Fc mRNA or ENPP1-Albumin mRNA) comprising polymeric film is then mounted on the stainless stent. The same process can be repeated for preparing a stent coated with a vector expressing ENPP1 polypeptide (ENPP1 or ENPP1-Fc or ENPP1-Albumin) by using 50 μg of vector DNA.

Some examples of ENPP1 comprising spray solutions are shown below, the spray solutions can be applied onto stents to create ENPP1 coated eluting stents. Optionally nonpolymeric carrier such as Vitamin E, Vitamin E acetate, Vitamin E succinate, oleic acid, peanut oil and cottonseed oil can be added to the spray solution improve the stability of ENPP1 agent.
(c) ENPP1 agent coating composition (C)—10 mg PCL (poly caprolactone) polymer and 100 μg ENPP1 mRNA is dissolved in sterile double distilled water at room temperature. 100 μl polymeric PCL solution comprising the ENPP1 mRNA (or ENPP1-Fc mRNA or ENPP1-Albumin mRNA) is sprayed onto a stent (6 mm×20 mm) using a semi-automated nebulizer apparatus. The nebulizer spray system provides means of rotating and traversing the length of the stent at a controlled rate. The traversing component of the apparatus contained a glass nebulizer system that applies nebulized polycaprolactone solution to the stent at a rate of 3 ml per minute. Once applied, the polymer coating is “reflowed” by application of 60° C. heated air for approximately 5 seconds. The process of reflowing the polymer provides better adherence to the stent surface. The same process can be repeated for preparing a stent coated with a vector expressing ENPP1 polypeptide (ENPP1 or ENPP1-Fc or ENPP1-Albumin) by using 50 μg of vector DNA.
(d) ENPP1 agent coating composition (D)-A 1% solution of uncured two-part silicone rubber is dissolved in trichloroethylene and then sprayed on to the stent using a nebulizer spray system as described above in (C). The coated stent is dried at room temperature for 15 minutes to allow the trichloroethylene to evaporate. The coated stent comprising silicone is heated in a vacuum oven for a period of four hours in order to crosslink the silicone coating. The coated stents are removed from the oven and allowed to cool for a period of 1 hour. 100 μg ENPP1 mRNA is dissolved in sterile double distilled water at room temperature. A volume of 100 μl of ENPP1 comprising spray solution is applied to the silicone coating of each stent in dropwise fashion. The crosslinked silicone absorbs the solution, where the solvent subsequently evaporates at room temperature, leaving behind the ENPP1 mRNA (or ENPP1-Fc mRNA or ENPP1-Albumin mRNA) entrapped within the silicone. The same process can be repeated for preparing a stent coated with a vector expressing ENPP1 polypeptide (ENPP1 or ENPP1-Fc or ENPP1-Albumin) by using 50 μg of vector DNA. The solvent subsequently evaporates at room temperature, leaving behind the ENPP1 encoding vector entrapped within the silicone.
(e) ENPP1 agent coating composition (E)-10 mg PCL (poly caprolactone) polymer and ENPP1 polypeptide (any one of ENPP1 or ENPP1-Fc or ENPP1-albumin) is dissolved in sterile double distilled water at room temperature to reach an ENPP1 polypeptide concentration of 10 mg/ml. 100 μl polymeric PCL solution comprising the ENPP1 polypeptide (10 mg/ml) is sprayed onto a stent as described in (C)
(f) ENPP1 agent coating composition (F)—The coated stent comprising silicone are prepared as discussed in (d). The coated stents are removed from the oven and allowed to cool for a period of 1 hour. ENPP1 polypeptide (ENPP1 or ENPP1-Fc or ENPP1-Albumin) is dissolved in a sterile double distilled water at room temperature to reach an ENPP1 polypeptide concentration of 10 mg/ml. A volume of 100 μl of ENPP1 comprising spray solution (10 mg/ml) is applied to the silicone coating of each stent in dropwise fashion. The crosslinked silicone absorbs the solution, where the solvent subsequently evaporates at room temperature, leaving behind the ENPP1 mRNA entrapped within the silicone.

Animal Model

Thirty 4-to-5-month-old juvenile pigs with the weight of 25-35 kg are procured from commercial sources. Thirty stainless steel vents are obtained from one or more commercial sources such as Abbot, Boston Scientific, Medtronic, Alvimedica, Lepu Medical Technology, Cordis, Balton or Biotronik. Thirty stainless steel stents thus obtained are coated with ENPP1 mRNA following the protocol shown above for coating. Thirty bare metal stents (BMSs) are obtained from Abbott to be used as control set. The ENPP1 coated stent is then sterilized using ethylene oxide, compressed, and mounted on a balloon angioplasty catheter. It is then deployed at a site in an artery using standard balloon angioplasty techniques. Same is done for the control set using bare metal stents.

All target sites are injured on Day 0 to create the in-stent restenosis model. The four peripheral artery sites are mapped using quantitative vascular angiography (QVA) as described in Example. The injury is created as described in Example 1.

The stents are randomly assigned and placed in the peripheral (bilateral profunda and superficial femoral) arteries (one stent per artery) of 30 pigs, one coated stent per pig. The pigs are then maintained on 100 mg aspirin per day. On Day 14, all vessels are assessed by angiography and Optical Coherence Tomography (OCT). Then the previously injured and stented artery sites were subjected to a re-injury event consisting of overstretch of the artery with a single inflation of a standard angioplasty balloon catheter at the same pressure/diameter as the original pre-stent injury was done (130% of the baseline reference diameter). Following re-injury interventions, final post-procedural angiography and OCT are recorded for select peripheral sites. Four weeks following the re-injury event on Day 14, arteries underwent repeat imaging with angiography and endovascular imaging (OCT). The treated peripheral segments are explanted and stored in 10% neutral buffered formalin.

Lumen area, Stent area, Neointimal thickness, Neointimal area and % of Stenosis is calculated for pigs with ENPP1 coated stents and pigs with bare metal stents. It is expected that the profunda arteries of animals treated with ENPP1-Fc coated stents would have a higher lumen area at compared to the vehicle control group treated with bare metal stents. The stent area is expected to be similar between both groups. Neointimal thickness and neointimal area are expected to be reduced in animals treated with ENPP1-Fc coated stents relative to the vehicle control animals with bare metal stents. In addition, animals treated with ENPP1-Fc are expected to have a markedly lower % area of stenosis as compared to the vehicle control group.

Thus, in situ administration of ENPP1 agent by using ENPP1 coated stents is expected to prevent and effectively treat myointimal proliferation and/or restenosis at the site of injury in peripheral arteries.

### Example 5—Preparation and Implantation of ENPP3 Coated Stent for the Treatment of Atherosclerotic Peripheral Blood Vessels

In this example, a juvenile pig animal model is used for implanting the ENPP3-coated stent to determine the efficacy of an ENPP3 coated stent to inhibit neointima formation, restenosis and inflammation. An ENPP3 agent coated stent is prepared and then implanted in peripheral artery. Four peripheral arterial sites are created for induction of neointimal response in each animal; one site was selected in each of four arteries (bilateral profunda and superficial femoral arteries) as described in Example 1.

Preparation of ENPP3 coated stent Any stent is amenable to be coated with ENPP3 agent. Common examples of commercial sources that sell stents for use include Abbot, Boston Scientific, Medtronic, Alvimedica, Lepu Medical Technology, Cordis, Balton or Biotronik. Peripheral arterial stents are shorter in length (12-18 mm) with a diameter range from 5-8 mm are commonly used for placement in iliac and femoral arteries. Henry et al. describes in detail the different types of stent lengths and diameters available for peripheral arteries (Henry et al., Tex Heart Inst J 2000; 27(2): 119-126.)

For example, a plain stent such as a bare metal stent can be converted to ENPP3 coated eluting stent by placing a polymeric film comprising ENPP3 mRNA inside the stent or by spraying a polymeric or nonpolymeric solution comprising ENPP3 mRNA or ENPP3 polypeptide on to the stent surface.

Some examples of ENPP3 polymeric film are shown below, the ENPP3 polymeric film can be placed inside stents to create ENPP3 coated eluting stents. Optionally nonpolymeric carrier such as Vitamin E, Vitamin E acetate, Vitamin E succinate, oleic acid, peanut oil and cottonseed oil can be added to the solution improve the stability of ENPP1 agent in the polymeric film
(g) ENPP3 agent coating composition (A)—10 mg PCL (poly caprolactone) polymer and 100 μg ENPP1 mRNA (or ENPP3-Fc mRNA or ENPP3-Albumin mRNA) are dissolved in sterile double distilled water at room temperature. The solution is poured onto a glass plate and the solvent is allowed to evaporate for 12-24 hours. After almost complete removal of the solvent, the ENPP3-loaded PCL film is removed from the glass plate and is cut to 1.5 cm by 1.5 cm size which is then further trimmed to fit the size of the stent. The ENPP3 mRNA (or ENPP3-Fc mRNA or ENPP3-Albumin mRNA) comprising polymeric film is then mounted on the stainless stent. The same process can be repeated for preparing a stent coated with a vector expressing ENPP1 polypeptide (ENPP3 or ENPP3-Fc or ENPP3-Albumin) by using 50 μg of vector DNA.
(h) ENPP3 agent coating composition (B)—10 mg EVA (ethylene-vinyl acetate) polymer and 100 μg ENPP3 mRNA (or ENPP3-Fc mRNA or ENPP3-Albumin mRNA) are dissolved in sterile double distilled water at room temperature. The solution is poured onto a glass plate and the solvent is allowed to evaporate for 12-24 hours. After almost complete removal of the solvent, the ENPP3-mRNA (or ENPP3-Fc mRNA or ENPP3-Albumin mRNA) loaded EVA film is removed from the glass plate and was cut to 1.5 cm by 1.5 cm size. The ENPP3 mRNA (or ENPP3-Fc mRNA or ENPP3-Albumin mRNA) comprising polymeric film is then mounted on the stainless stent. The same process can be repeated for preparing a stent coated with a vector expressing ENPP3 polypeptide (ENPP3 or ENPP3-Fc or ENPP3-Albumin) by using 50 μg of vector DNA.

Some examples of ENPP3 comprising spray solutions are shown below, the spray solutions can be applied onto stents to create ENPP3 coated eluting stents. Optionally nonpolymeric carrier such as Vitamin E, Vitamin E acetate, Vitamin E succinate, oleic acid, peanut oil and cottonseed oil can be added to the spray solution improve the stability of ENPP3 agent.
(i) ENPP3 agent coating composition (C)—10 mg PCL (poly caprolactone) polymer and 100 μg ENPP3 mRNA is dissolved in sterile double distilled water at room temperature. 100 μl polymeric PCL solution comprising the ENPP3 mRNA (or ENPP3-Fc mRNA or ENPP3-Albumin mRNA) is sprayed onto a stent (6 mm×20 mm) using a semi-automated nebulizer apparatus. The nebulizer spray system provides means of rotating and traversing the length of the stent at a controlled rate. The traversing component of the apparatus contained a glass nebulizer system that applies nebulized polycaprolactone solution to the stent at a rate of 3 ml per minute. Once applied, the polymer coating is “reflowed” by application of 60° C. heated air for approximately 5 seconds. The process of reflowing the polymer provides better adherence to the stent surface. The same process can be repeated for preparing a stent coated with a vector expressing ENPP3 polypeptide (ENPP3 or ENPP3-Fc or ENPP3-Albumin) by using 50 μg of vector DNA.
(j) ENPP3 agent coating composition (D)-A 1% solution of uncured two-part silicone rubber is dissolved in trichloroethylene and then sprayed on to the stent using a nebulizer spray system as described above in (C). The coated stent is dried at room temperature for 15 minutes to allow the trichloroethylene to evaporate. The coated stent comprising silicone is heated in a vacuum oven for a period of four hours in order to crosslink the silicone coating. The coated stents are removed from the oven and allowed to cool for a period of 1 hour. 100 μg ENPP3 mRNA is dissolved in sterile double distilled water at room temperature. A volume of 100 μl of ENPP3 comprising spray solution is applied to the silicone coating of each stent in dropwise fashion. The crosslinked silicone absorbs the solution, where the solvent subsequently evaporates at room temperature, leaving behind the ENPP3 mRNA (or ENPP3-Fc mRNA or ENPP3-Albumin mRNA) entrapped within the silicone. The same process can be repeated for preparing a stent coated with a vector expressing ENPP3 polypeptide (ENPP3 or ENPP3-Fc or ENPP3-Albumin) by using 50 μg of vector DNA. The solvent subsequently evaporates at room temperature, leaving behind the ENPP3 encoding vector entrapped within the silicone.
(k) ENPP3 agent coating composition (E)-10 mg PCL (poly caprolactone) polymer and ENPP3 polypeptide (any one of ENPP3 or ENPP3-Fc or ENPP3-albumin) is dissolved in sterile double distilled water at room temperature to reach an ENPP3 polypeptide concentration of 10 mg/ml. 100 μl polymeric PCL solution comprising the ENPP3 polypeptide (10 mg/ml) is sprayed onto a stent as described in (C)
(l) ENPP3 agent coating composition (F)—The coated stent comprising silicone are prepared as discussed in (d). The coated stents are removed from the oven and allowed to cool for a period of 1 hour. ENPP3 polypeptide (ENPP3 or ENPP3-Fc or ENPP3-Albumin) is dissolved in a sterile double distilled water at room temperature to reach an ENPP3 polypeptide concentration of 10 mg/ml. A volume of 100 μl of ENPP3 comprising spray solution (10 mg/ml) is applied to the silicone coating of each stent in dropwise fashion. The crosslinked silicone absorbs the solution, where the solvent subsequently evaporates at room temperature, leaving behind the ENPP3 mRNA entrapped within the silicone.

Animal Model

Thirty 4-to-5-month-old juvenile pigs with the weight of 25-35 kg are procured from commercial sources. Thirty stainless steel vents are obtained from one or more commercial sources such as Abbot, Boston Scientific, Medtronic, Alvimedica, Lepu Medical Technology, Cordis, Balton or Biotronik. Thirty stainless steel stents thus obtained are coated with ENPP1 mRNA following the protocol shown above for coating. Thirty bare metal stents (BMSs) are obtained from Abbott to be used as control set. The ENPP3 coated stent is then sterilized using ethylene oxide, compressed, and mounted on a balloon angioplasty catheter. It is then deployed at a site in an artery using standard balloon angioplasty techniques. Same is done for the control set using bare metal stents.

All target sites are injured on Day 0 to create the in-stent restenosis model. The four peripheral artery sites are mapped using quantitative vascular angiography (QVA) as described in Example. The injury is created as described in Example 1.

The stents are randomly assigned and placed in the peripheral (bilateral profunda and superficial femoral) arteries (one stent per artery) of 30 pigs, one coated stent per pig. The pigs are then maintained on 100 mg aspirin per day. On Day 14, all vessels are assessed by angiography and Optical Coherence Tomography (OCT). Then the previously injured and stented artery sites were subjected to a re-injury event consisting of overstretch of the artery with a single inflation of a standard angioplasty balloon catheter at the same pressure/diameter as the original pre-stent injury was done (130% of the baseline reference diameter). Following re-injury interventions, final post-procedural angiography and OCT are recorded for select peripheral sites. Four weeks following the re-injury event on Day 14, arteries underwent repeat imaging with angiography and endovascular imaging (OCT). The treated peripheral segments are explanted and stored in 10% neutral buffered formalin.

Lumen area, Stent area, Neointimal thickness, Neointimal area and % of Stenosis is calculated for pigs with ENPP3 coated stents and pigs with bare metal stents. It is expected that the profunda arteries of animals treated with ENPP3-Fc coated stents would have a higher lumen area at compared to the vehicle control group treated with bare metal stents. The stent area is expected to be similar between both groups. Neointimal thickness and neointimal area are expected to be reduced in animals treated with ENPP3-Fc coated stents relative to the vehicle control animals with bare metal stents. In addition, animals treated with ENPP3-Fc are expected to have a markedly lower % area of stenosis as compared to the vehicle control group.

Thus, in situ administration of ENPP3 agent by using ENPP3 coated stents is expected to prevent and effectively treat myointimal proliferation and/or restenosis at the site of injury in peripheral arteries.

### INCORPORATION BY REFERENCE

The disclosure of each and every U.S. and foreign patent and pending patent application and publication referred to herein is specifically incorporated herein by reference in its entirety, as are the contents of Sequence Listing and Figures.

### EQUIVALENTS

Those skilled in the art will recognize or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### OTHER EMBODIMENTS

From the foregoing description, it will be apparent that variations and modifications may be made to the disclosure described herein to adopt it to various usages and conditions, including the use of different signal sequences to express functional variants of ENPP1 or ENPP3 or combinations thereof in different viral vectors having different promoters or enhancers or different cell types known in art to treat any diseases characterized by the presence of pathological calcification or ossification are within the scope according to the disclosure. Other embodiments according to the disclosure are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Other embodiments are within the following claims.

1-118. (canceled)

119. A method of treating a subject having peripheral artery disease or reducing and/or preventing progression of vascular smooth muscle cell proliferation in a peripheral artery of a subject having peripheral artery disease, said method comprising: administering to the subject an amount of an ENPP1 or ENPP3 agent effective to treat said peripheral artery disease in said subject or to reduce and/or prevent progression of said vascular smooth muscle cell proliferation in said peripheral artery of said subject.

120. The method of claim 119, wherein the subject has common femoral artery disease, femoral-popliteal disease, tibial-peroneal disease, stage III, stage IV or stage IV, grade III peripheral artery disease.

121. The method of claim 119, wherein the ENPP1 agent or ENPP3 agent comprises: an ENPP1 or ENPP3 polypeptide; a nucleic acid encoding an ENPP1 or ENPP3 polypeptide; or a viral vector comprising a nucleic acid encoding an ENPP1 or ENPP3 polypeptide.

122. The method of claim 119, wherein the subject does not have a deficiency of ENPP1.

123. The method of claim 119, wherein the subject has a condition that requires surgery on said peripheral artery.

124. The method of claim 119, wherein the subject is a tobacco user, has hypertension, has elevated cholesterol or triglyceride levels, is a diabetic, has renal disease, or is obese.

125. The method of claim 121, wherein the ENPP1 or ENPP3 polypeptide comprises an extracellular domain, or a catalytic domain of ENPP1 or ENPP3.

126. The method of claim 121, wherein the ENPP1 polypeptide comprises amino acids 99 to 925 of SEQ ID NO:1, or wherein the ENPP3 polypeptide comprises amino acids 49-875 of SEQ ID NO: 7.

127. The method of claim 121, wherein the ENPP1 or ENPP3 polypeptide is fused to a heterologous protein that increases the circulating half-life of the ENPP1 or ENPP3 polypeptide in mammal.

128. The method of claim 127, wherein the heterologous protein is an Fc region of an immunoglobulin molecule, and wherein the immunoglobulin molecule is an IgG1 molecule; or an albumin molecule.

129. The method of claim 123, wherein the condition requiring surgery is due to a prior placement of a non-eluting arterial stent in said artery, or wherein the condition requiring is due to a prior placement of an eluting arterial stent in said artery which elutes therapeutic agents other than said ENPP1 or ENPP3 agent.

130. The stent of claim 121, wherein the ENPP1 agent is selected from a group consisting of: ENPP1, ENPP1-Fc, ENPP1-Albumin, and ENPP1 mRNA; or the ENPP3 agent is selected from a group consisting of: ENPP3, ENPP3-Fc, ENPP3-Albumin, and ENPP3 mRNA.

131. The method of claim 128, wherein the heterologous protein is carboxy terminal to the ENPP1 or ENPP3 peptide and/or wherein the ENPP1 or ENPP3 polypeptide comprises a linker, and wherein the linker separates the ENPP1 or ENPP3 polypeptide and the heterologous protein.

132. An arterial stent coated with an ENPP1 or ENPP3 agent for treating a subject having peripheral artery disease or for reducing and/or preventing progression of vascular smooth muscle cell proliferation in a peripheral artery of a subject having peripheral artery disease, wherein said stent is implanted into an artery of the subject, wherein said implanted stent is configured to release said ENPP1 or ENPP3 agent in an amount effective to treat said peripheral artery disease in said subject or to reduce and/or prevent progression of said vascular smooth muscle cell proliferation in said peripheral artery of said subject.

133. The stent of claim 132, wherein the subject has common femoral artery disease, femoral-popliteal disease, tibial-peroneal disease, stage III, stage IV or stage IV, grade III peripheral artery disease.

134. The stent of claim 132, wherein the ENPP1 agent or ENPP3 agent comprises: an ENPP1 or ENPP3 polypeptide; a nucleic acid encoding an ENPP1 or ENPP3 polypeptide; or a viral vector comprising a nucleic acid encoding an ENPP1 or ENPP3 polypeptide.

135. The stent of claim 132, wherein the subject does not have a deficiency of ENPP1.

136. The stent of claim 132, wherein the subject has a condition that requires surgery on said peripheral artery.

137. The stent of claim 132, wherein the subject is a tobacco user, has hypertension, has elevated cholesterol or triglyceride levels, is a diabetic, has renal disease, or is obese.

138. The stent of claim 134, wherein the ENPP1 or ENPP3 polypeptide comprises an extracellular domain, or a catalytic domain of ENPP1 or ENPP3.

139. The stent of claim 134, wherein the ENPP1 polypeptide comprises amino acids 99 to 925 of SEQ ID NO:1, or wherein the ENPP3 polypeptide comprises amino acids 49-875 of SEQ ID NO: 7.

140. The stent of claim 134, wherein the ENPP1 or ENPP3 polypeptide is fused to a heterologous protein functional to increase the circulating half-life of the ENPP1 or ENPP3 polypeptide in a mammal.

141. The stent of claim 140, wherein the heterologous protein is an Fc region of an immunoglobulin molecule, or an albumin molecule.

142. The stent of claim 136, wherein the condition requiring surgery is due to a prior placement of a non-eluting arterial stent in said artery, or wherein the condition requiring is due to a prior placement of an eluting arterial stent in said artery which elutes therapeutic agents other than said ENPP1 or ENPP3 agent.

143. The stent of claim 132, wherein the stent further comprises a carrier for said ENPP1 or ENPP3 agent and said stent is configured to release said ENPP1 or ENPP3 agent preferably at a rate of 1-10 μg/ml per day to said subject.

144. The stent of claim 143, wherein the carrier is non-reactive with the ENPP1 or ENPP3 agent and the carrier comprises a polymeric carrier or a nonpolymeric carrier, and said carrier is physically or chemically bound to the ENPP1 or ENPP3 agent.

145. The stent of claim 144, wherein the nonpolymeric carrier is selected from a group consisting of: Vitamin E, Vitamin E acetate, Vitamin E succinate, oleic acid, peanut oil and cottonseed oil.

146. The stent of claim 134, wherein the ENPP1 agent is selected from a group consisting of: ENPP1, ENPP1-Fc, ENPP1-Albumin, and ENPP1 mRNA; or the ENPP3 agent is selected from a group consisting of: ENPP3, ENPP3-Fc, ENPP3-Albumin, and ENPP3 mRNA.
